SEQUENCE LISTING (27 sequences)

SEQ ID NO: 1 - protein, 10 aa, synthetic construct
GYTFTDYNLH

SEQ ID NO: 2 - protein, 10 aa, synthetic construct
FIYPSNRITG

SEQ ID NO: 3 - protein, 8 aa, synthetic construct
SDVDYFDY

SEQ ID NO: 4 - protein, 15 aa, synthetic construct
RASQSVSTSTYSYMH

SEQ ID NO: 5 - protein, 7 aa, synthetic construct
YASNLES

SEQ ID NO: 6 - protein, 9 aa, synthetic construct
QHSWEIPLT

SEQ ID NO: 7 - protein, 117 aa, synthetic construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSS

SEQ ID NO: 8 - protein, 111 aa, synthetic construct
DIVLTQSPDSLAVSLGERATINCRASQSVSTSTYSYMHWYQQKPGQPPKLLIKYASNLESGVPDRFSGSGSGTDFTLTISSLQAEDVAVYYCQHSWEIPLTFGQGTKLEIK

SEQ ID NO: 9 - protein, 443 aa, synthetic construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 442 aa, synthetic construct
QVQLVQSGAEVKKPGASVKISCKASGYTFTDYNLHWVRQAPGQGLEWIGFIYPSNRITGYAQKFQGRATLTVDNSTSTAYMELSSLRSEDTAVYYCARSDVDYFDYWGQGTLLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 11 - protein, 218 aa, synthetic construct
DIVLTQSPDSLAVSLGERATINCRASQSVSTSTYSYMHWYQQKPGQPPKLLIKYASNLESGVPDRFSGSGSGTDFTLTISSLQAEDVAVYYCQHSWEIPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 5 aa, synthetic construct
NYEMN

SEQ ID NO: 13 - protein, 19 aa, synthetic construct
EIRLKSNNYVTNYAASVKG

SEQ ID NO: 14 - protein, 9 aa, synthetic construct
AGYYVPFAY

SEQ ID NO: 15 - protein, 12 aa, synthetic construct
TLSSQHSTYTIE

SEQ ID NO: 16 - protein, 11 aa, synthetic construct
LKKEGSHSTGD

SEQ ID NO: 17 - protein, 13 aa, synthetic construct
GVGHTIKEQFVYV

SEQ ID NO: 18 - protein, 120 aa, synthetic construct
EVQLVESGGGLVQPGGSLRLSCAGSGFTFSNYEMNWVRQAPGKGLEWVAEIRLKSNNYVTNYAASVKGRFTISRDDSKNSVYLQMNSLKTEDTGVYYCTRAGYYVPFAYWGQGTLVTVSS

SEQ ID NO: 19 - protein, 115 aa, synthetic construct
QLMLTQSPSASASLGASVKLTCTLSSQHSTYTIEWYQQQPGKGPRYLMELKKEGSHSTGDGIPDRFSGSSSGAERYLTISSLQSEDEADYYCGVGHTIKEQFVYVFGGGTKLTVL

SEQ ID NO: 20 - protein, 446 aa, synthetic construct
EVQLVESGGGLVQPGGSLRLSCAGSGFTFSNYEMNWVRQAPGKGLEWVAEIRLKSNNYVTNYAASVKGRFTISRDDSKNSVYLQMNSLKTEDTGVYYCTRAGYYVPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 21 - protein, 445 aa, synthetic construct
EVQLVESGGGLVQPGGSLRLSCAGSGFTFSNYEMNWVRQAPGKGLEWVAEIRLKSNNYVTNYAASVKGRFTISRDDSKNSVYLQMNSLKTEDTGVYYCTRAGYYVPFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 22 - protein, 221 aa, synthetic construct
QLMLTQSPSASASLGASVKLTCTLSSQHSTYTIEWYQQQPGKGPRYLMELKKEGSHSTGDGIPDRFSGSSSGAERYLTISSLQSEDEADYYCGVGHTIKEQFVYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 117 aa, Mus musculus
EVQLQQSGPELVKPGASVKISCKASGYTFTDYNLHWVKLSHGKSLEWIGFIYPSNGITGYNQKFKNKATLTVDNSSSTAYMELRSLTSEDSAVYYCARSTVDYFDYWGQGTTLTVSS

SEQ ID NO: 24 - protein, 111 aa, Mus musculus
DIVLTQSPASLAVSLGQRATMSCRASQSVSTSTYSYMHWYQQKPGQPPKLLIKYASNLESGVPARFSGSGSGTDFTLNIHPVEEEDTATYYCQHSWEIPLTFGAGTKLELK

SEQ ID NO: 25 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 26 - protein, 110 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCP

SEQ ID NO: 27 - protein, 10 aa, synthetic construct
FAGAGVTTER

(19) United States
(12) Patent Application Publication
(10) Pub. No.: US 2024/0329054 A1
(43) Pub. Date: Oct. 3, 2024

(54) SOLUBLE CD33 AS A BIOMARKER FOR ANTI-CD33 EFFICACY

(71) Applicant: Alector LLC, South San Francisco, CA (US)

(72) Inventors: Robert PAUL, San Francisco, CA (US); Michael F. WARD, San Francisco, CA (US); Felix Leejia YEH, Belmont, CA (US); Yijie LIAO, Pleasanton, CA (US); Herve RHINN, San Francisco, CA (US); Arnon ROSENTHAL, Woodside, CA (US)

(73) Assignee: Alector LLC, South San Francisco, CA (US)

(21) Appl. No.: 18/656,427

(22) Filed: May 6, 2024

Related U.S. Application Data
(63) Continuation of application No. PCT/US2022/079414, filed on Nov. 7, 2022.
(60) Provisional application No. 63/277,088, filed on Nov. 8, 2021.

(51) Int. Cl. G01N 33/68

(52) U.S. Cl. CPC G01N 33/6854; G01N 33/6896; G01N 2333/70503; G01N 2800/2821; G01N 2800/52

(57) ABSTRACT

The present disclosure relates generally to biomarkers of activity and/or therapeutic efficacy of anti-CD33 antibodies for the treatment of a disease or injury in an individual, as well as methods related thereto.

### CROSS-REFERENCE TO RELATED APPLICATIONS

This application claims the benefit of U.S. Provisional Application No. 63/277,088, filed Nov. 8, 2021, which is hereby incorporated by reference in its entirety.

### REFERENCE TO AN ELECTRONIC SEQUENCE LISTING

The contents of the electronic sequence listing (735022003840SEQLIST.xml; Size: 27,669 bytes; and Date of Creation: Nov. 7, 2022) are herein incorporated by reference in their entirety.

### TECHNICAL FIELD

Provided herein are biomarkers of activity and/or therapeutic efficacy of anti-CD33 antibodies for the treatment of diseases or injuries, as well as methods related thereto.

### BACKGROUND

Myeloid cell surface antigen CD33 precursor (CD33), also known as Siglec-3, is a type 1, immunoglobulin-like, transmembrane protein expressed on immune and hematopoietic cells, including immature and mature myeloid cells, dendritic cells, and microglial cells. (Crocker et al. (2007) Nat Rev Immunol. 7:255-266; McMillan and Crocker (2008) Carbohydr Res. 343:2050-2056; Von Gunten and Bochner (2008) Ann NY Acad Sci. 1143:61-82; Handgretinger et al. (1993) Immunol Lett. 37:223-228; and Hernández-Caselles et al. (2006) J Leukoc Biol. 79:46-58). CD33 contains an Ig-like C2-type (immunoglobulin-like) and an Ig-like V-type (immunoglobulin-like) extracellular domain, as well as two ITIM-like motifs in its cytoplasmic domain. Three alternatively spliced forms (isoforms) of CD33 have been identified, including a higher molecular weight variant, named CD33M and a smaller isoform CD33m that lacks the Ig-like V-type domain (the ligand-binding site), and the disulfide bond linking the V and C domains.

Genome-wide association studies (GWAS) performed on extended cohorts (e.g., thousands of individuals) have identified single nucleotide polymorphisms (SNPs) rs3865444^{CC} (AKA rs3826656) and rs3865444^{AA} in CD33 as genetic modulators of risk for late onset Alzheimer's disease (AD). In addition, carriers of the 245941977 allele, as well as carriers of the rs12459419^{CT} allele, which show over 25% reduction in expression of full-length CD33, display reduced Alzheimer's disease risk (Malik M. et al. (2015) Human Molecular Genetics, 1-14). This suggests that reduced expression or functionality of CD33 may be beneficial in Alzheimer's disease and other diseases associated with CD33 activity.

Therapeutic antibodies targeting CD33 are one solution to treating diseases associated with CD33 activity, such as Alzheimer's disease. Administration of antibodies, however, presents a challenge for therapeutic use. Generally, antibodies have limited oral bioavailability, so they are typically administered intravenously, subcutaneously, or intramuscularly (Ovacik, M and Lin, L, (2018) Clin Transl Sci 11, 540-552). Systemically administered antibodies normally exhibit a biphasic pharmacokinetic profile, being first distributed relatively quickly and then eliminated more slowly (Ovacik, M and Lin, L, (2018) Clin Transl Sci 11, 540-552). Circulation of systemically administered antibodies is typically confined to the vasculature and interstitial space (Ovacik, M and Lin, L, (2018) Clin Transl Sci 11, 540-552). Thus, determining whether a therapeutic antibody is reaching target locations in the body and having activity or therapeutic efficacy in the individual are important to understand responses to treatment with therapeutic antibodies.

Accordingly, there is a need in the art for identifying biomarkers of activity and/or therapeutic efficacy of anti-CD33 antibodies for the treatment of diseases associated with CD33 activity, such as Alzheimer's disease, and for methods of monitoring, assessing, guiding, or adjusting treatments with anti-CD33 antibodies based on such biomarkers of activity and/or therapeutic efficacy.

All references cited herein, including patents, patent applications and publications, are hereby incorporated by reference in their entirety.

### SUMMARY OF THE INVENTION

In one aspect, provided herein is a method for treating and/or delaying the progression of a disease or injury in an individual, comprising: (a) administering to an individual having a disease or injury a dose of an anti-CD33 antibody; (b) determining a level of soluble CD33 protein (sCD33) in a sample obtained from the individual after the individual has received the dose of the anti-CD33 antibody; (c) assessing activity of the anti-CD33 antibody in the individual based on the level of sCD33 in the sample, wherein the anti-CD33 antibody is determined to be active in the individual if the level of sCD33 in the sample is increased as compared to a level of sCD33 in a sample obtained from the individual prior to administration of the anti-CD33 antibody; (d) determining that the anti-CD33 antibody is active in the individual based, at least in part, on the assessing, and (e) administering one or more additional doses of the anti-CD33 antibody to the individual responsive to determining that the anti-CD33 antibody is active in the individual.

In another aspect, provided herein is a method of assessing activity of an anti-CD33 antibody in an individual having a disease or injury, the method comprising: (a) administering to an individual having a disease or injury a dose of an anti-CD33 antibody; (b) determining a level of soluble CD33 protein (sCD33) in a sample obtained from the individual after the individual has received the dose of the anti-CD33 antibody; (c) assessing activity of the anti-CD33 antibody in the individual based on the level of sCD33 in the sample, wherein the anti-CD33 antibody is determined to be active in the individual if the level of sCD33 in the sample is increased as compared to a level of sCD33 in a sample obtained from the individual prior to administration of the anti-CD33 antibody.

In another aspect, provided herein is a method of assessing activity of an anti-CD33 antibody in an individual having a disease or injury and being treated with an anti-CD33 antibody, the method comprising: (a) determining a level of soluble CD33 protein (sCD33) in a sample obtained from an individual having a disease or injury and being treated with an anti-CD33 antibody, wherein the sample is obtained after the individual has received a dose of the anti-CD33 antibody; (b) assessing activity of the anti-CD33 antibody in the individual based on the level of sCD33 in the sample, wherein the anti-CD33 antibody is determined to be active in the individual if the level of sCD33 in the sample is increased as compared to a level of sCD33 in a sample obtained from the individual prior to the start of treatment with the anti-CD33 antibody.

In another aspect, provided herein is a method of monitoring treatment of an individual having a disease or injury and being treated with an anti-CD33 antibody, the method comprising: (a) determining a level of soluble CD33 protein (sCD33) in a sample obtained from an individual having a disease or injury and being treated with an anti-CD33 antibody, wherein the sample is obtained at a first time point after the individual has received a dose of the anti-CD33 antibody; (b) determining a level of sCD33 in one or more additional samples obtained from the individual at a one or more time points after the first time point; (c) assessing activity of the anti-CD33 antibody in the individual based on the level of sCD33 in the sample and/or in the one or more additional samples, wherein the anti-CD33 antibody is determined to be active in the individual if the level of sCD33 in the sample and/or in the one or more additional samples is increased as compared to a level of sCD33 in a sample obtained from the individual prior to the start of treatment with the anti-CD33 antibody.

In some embodiments which may be combined with any of the preceding aspects or embodiments, the method comprises determining that the anti-CD33 antibody is active in the individual based, at least in part, on the assessing. In some embodiments, the method further comprises providing information on the activity of the anti-CD33 antibody in the individual, wherein said information indicates that the anti-CD33 antibody is determined to be active in the individual. In some embodiments, said information comprises a recommendation to administer one or more additional doses of the anti-CD33 antibody to the individual based, at least in part, on the determination that the anti-CD33 antibody is active in the individual. In some embodiments, the method further comprises administering one or more additional doses of the anti-CD33 antibody to the individual responsive to the determination that the anti-CD33 antibody is active in the individual.

In another aspect, provided herein is a method for assessing therapeutic efficacy of an anti-CD33 antibody for the treatment of a disease or injury in an individual, the method comprising: (a) determining a level of soluble CD33 protein (sCD33) in a sample obtained from an individual having a disease or injury and being treated with an anti-CD33 antibody, wherein the sample is obtained after the individual has received a dose of the anti-CD33 antibody; and (b) assessing therapeutic efficacy of the anti-CD33 antibody in the individual based on the level of sCD33 in the sample, wherein the anti-CD33 antibody is determined to have therapeutic efficacy in the individual if the level of sCD33 in the sample is increased as compared to a level of sCD33 in a sample obtained from the individual prior to the start of treatment with the anti-CD33 antibody. In some embodiments, the method further comprises determining that the anti-CD33 antibody has therapeutic efficacy in the individual based, at least in part, on the assessing. In some embodiments, the method further comprises providing information on the therapeutic efficacy of the anti-CD33 antibody in the individual, wherein said information indicates that the anti-CD33 antibody is determined to have therapeutic efficacy in the individual. In some embodiments, said information comprises a recommendation to administer one or more additional doses of the anti-CD33 antibody to the individual responsive to the determination that the anti-CD33 antibody has therapeutic efficacy in the individual. In some embodiments, the method further comprises administering to the individual one or more additional doses of the anti-CD33 antibody responsive to the determination that the anti-CD33 antibody has therapeutic efficacy in the individual.

In another aspect, provided herein is a method for guiding treatment with an anti-CD33 antibody in an individual having a disease or injury, the method comprising: (a) administering a dose of an anti-CD33 antibody to an individual having a disease or injury; (b) determining a level of soluble CD33 protein (sCD33) in a sample obtained from the individual after the individual has received the dose of the anti-CD33 antibody, wherein: (i) presence of an increase in the level of sCD33 in the sample indicates that treatment with the anti-CD33 antibody should continue, or (ii) absence of an increase in the level of sCD33 in the sample indicates that treatment with the anti-CD33 antibody should continue at a higher dose of the anti-CD33 antibody as compared to the dose administered in step (a), wherein the increase in the level of sCD33 in the sample is determined as compared to a level of sCD33 in a sample obtained from the individual prior to administration of the anti-CD33 antibody. In some embodiments, the method further comprises providing information on the presence or absence of the increase in the level of sCD33 in the sample. In some embodiments, said information comprises: (a) a recommendation to administer one or more additional doses of the anti-CD33 antibody to the individual based, at least in part, on the presence of an increase in the level of sCD33 in the sample; or (b) a recommendation to administer one or more additional higher doses of the anti-CD33 antibody to the individual based, at least in part, on the absence of an increase in the level of sCD33 in the sample. In some embodiments, the method further comprises: (a) administering one or more additional doses of the anti-CD33 antibody to the individual responsive to the presence of an increase in the level of sCD33 in the sample; or (b) administering one or more additional higher doses of the anti-CD33 antibody to the individual responsive to the absence of an increase in the level of sCD33 in the sample.

In another aspect, provided herein is a method for adjusting treatment of an individual having a disease or injury and being treated with an anti-CD33 antibody, the method comprising: (a) determining a level of soluble CD33 protein (sCD33) in a sample obtained from an individual having a disease or injury and being treated with one or more doses of an anti-CD33 antibody, wherein the sample is obtained after the individual has received a dose of the anti-CD33 antibody, wherein: (i) presence of an increase in the level of sCD33 in the sample indicates that treatment with the anti-CD33 antibody should continue, or (ii) absence of an increase in the level of sCD33 in the sample indicates that treatment with the anti-CD33 antibody should continue at a higher dose of the anti-CD33 antibody as compared to the one or more doses of anti-CD33 antibody being administered to the individual, wherein the increase in the level of sCD33 in the sample is determined as compared to a level of sCD33 in a sample obtained from the individual prior to administration of the anti-CD33 antibody; and (b) adjusting the treatment of the individual based, at least in part, on determining presence or absence of the increase in the level of sCD33 in the sample. In some embodiments, the adjusting comprises: (a) administering one or more additional doses of the anti-CD33 antibody to the individual responsive to the presence of an increase in the level of sCD33 in the sample; or (b) administering one or more additional higher doses of the anti-CD33 antibody to the individual responsive to the absence of an increase in the level of sCD33 in the sample.

In another aspect, provided herein is a method for providing information on the activity of an anti-CD33 antibody in an individual having a disease or injury and being administered an anti-CD33 antibody, the method comprising: (a) determining a level of soluble CD33 protein (sCD33) in a sample obtained from an individual having a disease or injury and being administered an anti-CD33 antibody, wherein the sample is obtained after the individual has received a dose of the anti-CD33 antibody; and (b) assessing activity of the anti-CD33 antibody in the individual based on the level of sCD33 in the sample, wherein the anti-CD33 antibody is determined to be active in the individual if the level of sCD33 in the sample is increased as compared to a level of sCD33 in a sample obtained from the individual prior to the start of treatment with the anti-CD33 antibody; and (c) providing information on the activity of the anti-CD33 antibody in the individual based, at least in part, on the assessing. In some embodiments, the method further comprises determining that the anti-CD33 antibody is active in the individual based, at least in part, on the assessing. In some embodiments, said information indicates that the anti-CD33 antibody is determined to be active in the individual. In some embodiments, said information comprises a recommendation to administer one or more additional doses of the anti-CD33 antibody to the individual.

In some embodiments which may be combined with any of the preceding aspects or embodiments, the sample or the one or more additional samples obtained from the individual are a sample of blood or cerebrospinal fluid. In some embodiments, the sample of blood is a sample of whole blood, plasma, or serum. In some embodiments which may be combined with any of the preceding aspects or embodiments, the sample or the one or more additional samples obtained from the individual are a sample of cerebrospinal fluid. In some embodiments which may be combined with any of the preceding aspects or embodiments, the sample or the one or more additional samples obtained from the individual are a sample of plasma. In some embodiments which may be combined with any of the preceding aspects or embodiments, the sample or the one or more additional samples obtained from the individual are obtained at about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or more, days after the individual has received a dose of the anti-CD33 antibody. In some embodiments which may be combined with any of the preceding aspects or embodiments, the method further comprises obtaining the sample, or the one or more samples, from the individual.

In some embodiments which may be combined with any of the preceding aspects or embodiments, the level of sCD33 in the sample, or in the one or more additional samples, obtained from the individual is determined using immunoblotting, mass spectrometry, an aptamer-based method, flow cytometry, an immunoassay, an electrochemiluminescence-based method, a proximity extension assay, or enzyme-linked immunosorbent assay (ELISA).

In some embodiments which may be combined with any of the preceding aspects or embodiments, the disease or injury is selected from dementia, frontotemporal dementia, Alzheimer's disease, vascular dementia, mixed dementia, or taupathy disease. In some embodiments, the disease or injury is Alzheimer's disease. In some embodiments, the disease or injury is mild to moderate Alzheimer's disease. In some embodiments, the individual has a clinical diagnosis of probable Alzheimer's disease dementia based on National Institute on Aging Alzheimer's Association criteria. In some embodiments, the individual has a Mini-Mental State Examination (MMSE) score of between about 16 points to about 28 points prior to the start of treatment with the anti-CD33 antibody. In some embodiments, the individual has a Clinical Dementia Rating-Global Score (CDR-GS) of about 0.5, about 1.0, or about 2.0 prior to the start of treatment with the anti-CD33 antibody. In some embodiments, the individual has a positive amyloid-PET scan by qualitative read prior to the start of treatment with the anti-CD33 antibody. In some embodiments, the individual has been taking a stable dose of a cholinesterase inhibitor and/or a memantine therapy for Alzheimer's disease for at least about 4 weeks prior to the start of treatment with the anti-CD33 antibody.

In some embodiments which may be combined with any of the preceding aspects or embodiments, the individual is a human. In some embodiments, the individual is a human adult.

In some embodiments which may be combined with any of the preceding aspects or embodiments, said information is provided as a report in an electronic, web-based, or paper form. In some embodiments which may be combined with any of the preceding aspects or embodiments, said information is provided to the individual, a caregiver, a physician, a hospital, a clinic, a third-party payer, an insurance company or a government office.

In some embodiments which may be combined with any of the preceding aspects or embodiments, the anti-CD33 antibody binds specifically to a mammalian or a human CD33 protein. In some embodiments which may be combined with any of the preceding aspects or embodiments, the anti-CD33 antibody binds specifically to a human CD33 protein. In some embodiments which may be combined with any of the preceding aspects or embodiments, the anti-CD33 antibody is a humanized antibody, a bispecific antibody, a murine antibody, a monoclonal antibody, a multivalent antibody, a conjugated antibody, or a chimeric antibody. In some embodiments which may be combined with any of the preceding aspects or embodiments, the anti-CD33 antibody is a monoclonal antibody. In some embodiments which may be combined with any of the preceding aspects or embodiments, the anti-CD33 antibody is a bispecific antibody recognizing a first antigen and a second antigen, wherein the first antigen is a human or mammalian CD33 protein. In some embodiments, the second antigen is: an antigen facilitating transport across the blood-brain-barrier; an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30 (A), a protein transduction domain, TAT, Syn-B, penetratin, a poly-arginine peptide, an angiopep peptide, or ANG1005; a disease-causing agent selected from disease-causing peptides or proteins, or disease-causing nucleic acids, wherein the disease-causing peptides or proteins are selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, or proline-arginine (PR) repeat peptides, and the disease-causing nucleic acids are antisense GGCCCC (G2C4) repeat-expansion RNA; ligands and/or proteins expressed on immune cells, wherein the ligands and/or proteins are selected from CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, Siglec-5, Siglec-7, Siglec-9, Siglec-11, SirpA, CD47, CSF1-receptor, or phosphatidylserine; or a protein, lipid, polysaccharide, or glycolipid expressed on one or more tumor cells. In some embodiments which may be combined with any of the preceding aspects or embodiments, the anti-CD33 antibody is of the IgG class, the IgM class, or the IgA class. In some embodiments which may be combined with any of the preceding aspects or embodiments, the anti-CD33 antibody is of the IgG class and has an IgG1, IgG2, IgG3, or IgG4 isotype. In some embodiments, the anti-CD33 antibody has an IgG4 isotype, and wherein the antibody comprises an S228P amino acid substitution at residue position 228, an F234A amino acid substitution at residue position 234, and an L235A amino acid substitution at residue position 235, wherein the numbering of the residue position is according to EU numbering. In some embodiments, the anti-CD33 antibody has an IgG2 isotype. In some embodiments, the anti-CD33 antibody comprises one or more amino acid substitutions in the Fc region at a residue position selected from: C127S, L234A, L234F, L235A, L235E, S267E, K322A, L328F, A330S, P331S, E345R, E430G, S440Y, or any combination thereof, wherein the numbering of the residues is according to EU or Kabat numbering. In some embodiments, the Fc region comprises an amino acid substitution at position E430G, wherein the numbering of the residue position is according to EU numbering; the Fc region comprises an amino acid substitution at positions L243A, L235A, and P331A, wherein the numbering of the residue position is according to EU numbering; the Fc region comprises an amino acid substitution at positions L243A, L235A, P331A, and E430G, wherein the numbering of the residue position is according to EU numbering; the Fc region comprises an amino acid substitution at positions K322A and E430G, wherein the numbering of the residue position is according to EU numbering; the Fc region comprises an amino acid substitution at positions P331S and E430G, wherein the numbering of the residue position is according to EU numbering; the Fc region comprises an amino acid substitution at positions A330S, P331S, and E430G, wherein the numbering of the residue position is according to EU numbering; the Fc region comprises an amino acid substitution at positions K322A, A330S, and P331S, wherein the numbering of the residue position is according to EU numbering; the Fc region comprises an amino acid substitution at positions K322A, P331S, and E430G, wherein the numbering of the residue position is according to EU numbering; the Fc region comprises an amino acid substitution at position E430G, wherein the numbering of the residue position is according to EU numbering; the Fc region comprises an amino acid substitution at positions A330S, P331S, and E430G, wherein the numbering of the residue position is according to EU numbering; the Fc region comprises an amino acid substitution at positions S267E and L328F, wherein the numbering of the residue position is according to EU numbering; the Fc region comprises an amino acid substitution at position C127S, wherein the numbering of the residue position is according to EU numbering; the Fc region comprises an amino acid substitution at positions E345R, E430G and S440Y, wherein the numbering of the residue position is according to EU numbering; the Fc region comprises an amino acid substitution at position P331S, wherein the numbering of the residue position is according to EU numbering; or the Fc region comprises an amino acid substitution at positions L234A, L235A, P331S, wherein the numbering of the residue positions is according to EU numbering. In some embodiments, the anti-CD33 antibody has a human IgG1 isotype and comprises one or more amino acid substitutions in the Fc region at a residue position selected from: N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, T394D, or any combination thereof, wherein the numbering of the residues is according to EU numbering, or comprises an amino acid deletion in the Fc region at a position corresponding to glycine 236; the anti-CD33 antibody has a human IgG1 isotype and comprises an IgG2 isotype heavy chain constant domain 1 (CH1) and hinge region, wherein the IgG2 isotype CH1 and hinge region comprises the amino acid sequence of ASTKGPSVFP LAPCSRSTSE STAALGCLVK DYFPEPVTVS WNSGALTSGVHTFPAVLQSS GLYSLSSVVT VPSSNFGTQT YTCNVDHKPS NTKVDKTVERKCCVECPPCP (SEQ ID NO: 26), and wherein the antibody Fc region comprises a S267E amino acid substitution, or a L328F amino acid substitution, or both, and/or a N297A or N297Q amino acid substitution, wherein the numbering of the residues is according to EU numbering; the anti-CD33 antibody has a human IgG2 isotype and comprises one or more amino acid substitutions in the Fc region at a residue position selected from: P238S, V234A, G237A, H268A, H268Q, V309L, A330S, P331S, C214S, C232S, C233S, S267E, L328F, M252Y, S254T, T256E, H268E, N297A, N297Q, A330L, or any combination thereof, wherein the numbering of the residues is according to EU numbering; the anti-CD33 antibody has a human IgG4 isotype and comprises one or more amino acid substitutions in the Fc region at a residue position selected from: L235A, G237A, S228P, L236E, S267E, E318A, L328F, M252Y, S254T, T256E, E233P, F234V, L234A/F234A, S228P, S241P, L248E, T394D, N297A, N297Q, L235E, or any combination thereof, wherein the numbering of the residues is according to EU numbering; or the anti-CD33 antibody has a hybrid IgG2/4 isotype, and wherein the antibody comprises an amino acid sequence comprising amino acids 118 to 260 of human IgG2 and amino acids 261 to 447 of human IgG4, wherein the numbering of the residues is according to EU or Kabat numbering. In some embodiments which may be combined with any of the preceding aspects or embodiments, the anti-CD33 antibody is an antibody fragment. In some embodiments, the antibody fragment binds to an epitope comprising amino acid residues on a human or mammalian CD33 protein. In some embodiments which may be combined with any of the preceding aspects or embodiments, the anti-CD33 antibody is an antibody fragment that binds to a human CD33 protein. In some embodiments, the antibody fragment is an Fab, Fab′, Fab′-SH, F(ab′)₂, Fv, or scFv fragment. In some embodiments, the antibody fragment is cross-linked to a second antibody fragment that binds to a human or mammalian CD33 protein. In some embodiments, the human or mammalian CD33 protein is a wild-type human or mammalian CD33 protein, a naturally occurring variant of human or mammalian CD33, or a disease variant of human or mammalian CD33. In some embodiments which may be combined with any of the preceding aspects or embodiments, the CD33 protein is expressed on one or more cells selected from human dendritic cells, human macrophages, human monocytes, human osteoclasts, human neutrophils, human T cells, human T helper cell, human cytotoxic T cells, human granulocytes, or human microglia.

In some embodiments which may be combined with any of the preceding aspects or embodiments, the anti-CD33 antibody has a dissociation constant (K_{D}) for human CD33 that ranges from about 2 nM to about 200 PM and wherein the Kp is determined by BioLayer Interferometry. In some embodiments which may be combined with any of the preceding aspects or embodiments, the anti-CD33 antibody reduces cell surface levels of a CD33 protein. In some embodiments the CD33 protein is expressed on the surface of human dendritic cells or monocytes. In some embodiments, the anti-CD33 antibody reduces cell surface levels of CD33 in vitro. In some embodiments, the anti-CD33 antibody reduces cell surface levels of CD33 in vitro with a half maximal effective concentration (EC50) that is less than 150 pM, or less than 40 pM, as measured by flow cytometry. In some embodiments which may be combined with any of the preceding aspects or embodiments, the anti-CD33 antibody increases expression of one or more disease-associated microglia (DAM) markers. In some embodiments, the one or more DAM markers is ApoE3, Tyrobp, B2m, Trem2, Cst7, Cts1, Lp1, Cd9, Ax1, Csf1, Ccl6, Itgax, Clec7a, Lilrb4, Timp2, or any combination thereof. In some embodiments, the anti-CD33 antibody inhibits cell surface clustering of CD33. In some embodiments which may be combined with any of the preceding aspects or embodiments, the anti-CD33 antibody inhibits one or more CD33 activities, wherein the one or more CD33 activities are selected from: CD33 binding to sialic acid-containing glycoproteins, or sialic acid-containing glycolipids, or both; modulated expression of one or more anti-inflammatory cytokines, wherein the one or more anti-inflammatory cytokines are selected from IL-10, TGF-beta, or IL-6; modulated expression of one or more anti-inflammatory cytokines in one or more cells selected from macrophages, dendritic cells, bone marrow-derived dendritic cells, monocytes, granulocytes, neutrophils, and microglial cells; modulated expression of one or more pro-inflammatory cytokines, wherein the one or more pro-inflammatory cytokines are selected from IL-1B, TNF-α, IL-6, IL-8, IL-12, or MCP-1; modulated expression of one or more pro-inflammatory cytokines in one or more cells selected from macrophages, dendritic cells, bone marrow-derived dendritic cells, monocytes, granulocytes, neutrophils, or microglial cells; modulated expression of one or more proteins selected from C1qa, C1qB, C1qC, C1s, CIR, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, CD14, CD16, HLA-DR, or CCR2; reducing T cell proliferation induced by one or more cells selected from dendritic cells, bone marrow-derived dendritic cells, monocytes, microglia, M1 microglia, activated M1 microglia, M2 microglia, macrophages, M1 macrophages, activated M1 macrophages, or M2 macrophages; decreasing proliferation of one or more cells selected from dendritic cells, bone marrow-derived dendritic cells, macrophages, M1 macrophages, activated M1 macrophages, M2 macrophages, monocytes, granulocytes, neutrophils, microglia, M1 microglia, activated M1 microglia, or M2 microglia; decreasing one or more functions of one or more cells selected from dendritic cells, bone marrow-derived dendritic cells, macrophages, M1 macrophages, activated M1 macrophages, M2 macrophages, monocytes, granulocytes, neutrophils, microglia, M1 microglia, activated M1 microglia, or M2 microglia; inhibition of phagocytosis of one or more of apoptotic neurons, nerve tissue debris, dysfunctional synapses, non-nerve tissue debris, bacteria, other foreign bodies, disease-causing proteins, disease-causing peptides, and disease-causing nucleic acids, wherein the disease-causing nucleic acids are antisense GGCCCC (G2C4) repeat-expansion RNA, the disease-causing proteins are selected from amyloid beta, oligomeric amyloid beta, amyloid beta plaques, amyloid precursor protein or fragments thereof, Tau, LAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, or proline-arginine (PR) repeat peptides; and binding to CD33 ligand on cells selected from neutrophils, dendritic cells, bone marrow-derived dendritic cells, monocytes, microglia, or macrophages. In some embodiments which may be combined with any of the preceding aspects or embodiments, the anti-CD33 antibody binds an inhibitory Fc receptor. In some embodiments, the inhibitory Fc receptor is inhibitory Fc-gamma receptor IIB (FcγIIB). In some embodiments which may be combined with any of the preceding aspects or embodiments, the anti-CD33 antibody increases phagocytosis by microglia. In some embodiments which may be combined with any of the preceding aspects or embodiments, the anti-CD33 antibody inhibits interaction between a human or mammalian CD33 protein and one or more CD33 ligands.

In some embodiments which may be combined with any of the preceding aspects or embodiments, the anti-CD33 antibody is used in combination with one or more antibodies that specifically bind a disease-causing agent selected from disease-causing peptides, disease-causing proteins, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, or any combination thereof; or with one or more antibodies that bind an immunomodulatory protein selected from CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR3, DR5, CD39, CD70, CD73, LAG3, TREM1, TREM2, Siglec-5, Siglec-7, Siglec-9, Siglec-11, SirpA, CD47, CSF1-receptor, phosphatidylserine, disease-causing nucleic acids, antisense GGCCCC (G2C4) repeat-expansion RNA, or any combination thereof.

In some embodiments which may be combined with any of the preceding aspects or embodiments, the anti-CD33 antibody comprises: (a) a heavy chain variable region comprising: an HVR-H1 comprising the amino acid sequence GYTFTDYNLH (SEQ ID NO: 1), or an amino acid sequence with at least about 90% homology to the amino acid sequence of SEQ ID NO: 1; an HVR-H2 comprising the amino acid sequence FIYPSNRITG (SEQ ID NO: 2), or an amino acid sequence with at least about 90% homology to the amino acid sequence of SEQ ID NO: 2; and an HVR-H3 comprising the amino acid sequence SDVDYFDY (SEQ ID NO: 3), or an amino acid sequence with at least about 90% homology to the amino acid sequence of SEQ ID NO: 3; and (b) a light chain variable region comprising: an HVR-L1 comprising the amino acid sequence RASQSVSTSTYSYMH (SEQ ID NO: 4), or an amino acid sequence with at least about 90% homology to the amino acid sequence of SEQ ID NO: 4; an HVR-L2 comprising the amino acid sequence YASNLES (SEQ ID NO: 5), or an amino acid sequence with at least about 90% homology to the amino acid sequence of SEQ ID NO: 5, and an HVR-L3 comprising the amino acid sequence QHSWEIPLT (SEQ ID NO: 6), or an amino acid sequence with at least about 90% homology to the amino acid sequence of SEQ ID NO: 6. In some embodiments, the anti-CD33 antibody comprises a heavy chain variable region comprising the amino acid sequence of SEQ ID NO: 7, and a light chain variable region comprising the amino acid sequence of SEQ ID NO: 8. In some embodiments, the anti-CD33 antibody has an IgG2 isotype. In some embodiments, the anti-CD33 antibody has a human IgG2 isotype. In some embodiments which may be combined with any of the preceding aspects or embodiments, the anti-CD33 antibody comprises a heavy chain comprising the amino acid sequence of SEQ ID NO: 9 or SEQ ID NO: 10, and a light chain comprising the amino acid sequence of SEQ ID NO: 11.

In some embodiments which may be combined with any of the preceding aspects or embodiments, the dose, or the one or more doses, of the anti-CD33 antibody comprise a dose of at least about 1.6 mg/kg of the anti-CD33 antibody. In some embodiments, the dose, or the one or more doses, of the anti-CD33 antibody comprise a dose of between about 1.6 mg/kg and about 15 mg/kg of the anti-CD33 antibody. In some embodiments, the dose, or the one or more doses, of the anti-CD33 antibody comprise a dose of about 1.6 mg/kg, about 5 mg/kg, about 7.5 mg/kg, about 10 mg/kg, or about 15 mg/kg of the anti-CD33 antibody. In some embodiments, the dose, or the one or more doses, of the anti-CD33 antibody are administered to the individual once every two weeks, once every three weeks, once every four weeks, once every five weeks, once every six weeks, once every seven weeks, once every eight weeks, once every nine weeks, once every ten weeks, once every eleven weeks, or once every twelve weeks. In some embodiments, the dose, or the one or more doses, of the anti-CD33 antibody comprise a dose of: about 1.6 mg/kg administered once every two weeks; about 1.6 mg/kg administered once every four weeks; about 5 mg/kg administered once every two weeks; about 5 mg/kg administered once every four weeks; about 5 mg/kg administered once every five weeks; about 5 mg/kg administered once every six weeks; about 5 mg/kg administered once every seven weeks; about 5 mg/kg administered once every eight weeks; about 15 mg/kg administered once every four weeks; about 15 mg/kg administered once every five weeks; about 15 mg/kg administered once every six weeks; about 15 mg/kg administered once every seven weeks; or about 15 mg/kg administered once every eight weeks.

In some embodiments which may be combined with any of the preceding aspects or embodiments, the anti-CD33 antibody comprises: (a) a heavy chain variable region comprising: an HVR-H1 comprising the amino acid sequence NYEMN (SEQ ID NO: 12), or an amino acid sequence with at least about 90% homology to the amino acid sequence of SEQ ID NO: 12; an HVR-H2 comprising the amino acid sequence EIRLKSNNYVTNYAASVKG (SEQ ID NO: 13), or an amino acid sequence with at least about 90% homology to the amino acid sequence of SEQ ID NO: 13; and an HVR-H3 comprising the amino acid sequence AGYYVPFAY (SEQ ID NO: 14), or an amino acid sequence with at least about 90% homology to the amino acid sequence of SEQ ID NO: 14; and (b) a light chain variable region comprising: an HVR-L1 comprising the amino acid sequence TLSSQHSTYTIE (SEQ ID NO: 15), or an amino acid sequence with at least about 90% homology to the amino acid sequence of SEQ ID NO: 15; an HVR-L2 comprising the amino acid sequence LKKEGSHSTGD (SEQ ID NO: 16), or an amino acid sequence with at least about 90% homology to the amino acid sequence of SEQ ID NO: 16, and an HVR-L3 comprising the amino acid sequence GVGHTIKEQFVYV (SEQ ID NO: 17), or an amino acid sequence with at least about 90% homology to the amino acid sequence of SEQ ID NO: 17. In some embodiments which may be combined with any of the preceding aspects or embodiments, the anti-CD33 antibody comprises a heavy chain variable region comprising the amino acid sequence of SEQ ID NO: 18, and a light chain variable region comprising the amino acid sequence of SEQ ID NO: 19. In some embodiments which may be combined with any of the preceding aspects or embodiments, the anti-CD33 antibody comprises an IgG2 isotype. In some embodiments which may be combined with any of the preceding aspects or embodiments, the anti-CD33 antibody comprises a human IgG2 isotype. In some embodiments which may be combined with any of the preceding aspects or embodiments, the anti-CD33 antibody comprises a heavy chain comprising the amino acid sequence of SEQ ID NO: 20 or 21, and a light chain comprising the amino acid sequence of SEQ ID NO: 22.

In some embodiments which may be combined with any of the preceding aspects or embodiments, the sample or the one or more additional samples obtained from the individual are a cerebrospinal fluid sample, and wherein sCD33 levels in the sample or in the one or more additional samples obtained from the individual are increased by at least about 100%, at least about 125%, at least about 150%, at least about 175%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, or more, as compared to a level of sCD33 in a sample obtained from the individual prior to administration of the anti-CD33 antibody. In some embodiments, the increase in the level of SCD33 is present for at least about 8 days, at least about 18 days, at least about 36 days, at least about 43 days, at least about 50 days, or at least about 64 days after administration of a dose of the anti-CD33 antibody.

In some embodiments which may be combined with any of the preceding aspects or embodiments, the method further comprises measuring a concentration of the anti-CD33 antibody in a sample obtained from the individual after the individual has received one or more doses of the anti-CD33 antibody. In some embodiments, the sample is a sample of plasma, serum, or cerebrospinal fluid.

In some embodiments which may be combined with any of the preceding aspects or embodiments, the half-life of the anti-CD33 antibody in serum is at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

In some embodiments which may be combined with any of the preceding aspects or embodiments, the method further comprises measuring expression of a CD33 protein on the surface of one or more cells in a sample obtained from the individual after the individual has received one or more doses of the anti-CD33 antibody. In some embodiments, the sample is a blood sample. In some embodiments which may be combined with any of the preceding aspects or embodiments, the anti-CD33 antibody reduces cell surface levels of a CD33 protein on one or more cells in the individual. In some embodiments, the one or more cells are monocytes.

In some embodiments which may be combined with any of the preceding aspects or embodiments, the anti-CD33 antibody reduces cell surface levels of a CD33 protein on one or more monocytes in the individual by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, as compared to cell surface levels of the CD33 protein on one or more monocytes prior to administration of the anti-CD33 antibody. In some embodiments, the reduction of cell surface levels of the CD33 protein is present for at least about 1 day, at least about 2 days, at least about 5 days, at least about 8 days, at least about 13 days, at least about 15 days, at least about 22 days, at least about 29 days, at least about 30 days, at least about 36 days, at least about 43 days, at least about 50 days, at least about 57 days, at least about 64 days, at least about 78 days, at least about 106 days, or at least about 141 days after administration of a dose of the anti-CD33 antibody.

In some embodiments which may be combined with any of the preceding aspects or embodiments, the method further comprises measuring levels of one or more biomarkers of neuroinflammation in a sample obtained from the individual after the individual has received one or more doses of the anti-CD33 antibody.

In some embodiments which may be combined with any of the preceding aspects or embodiments, the method further comprises measuring levels of one or more biomarkers of Alzheimer's disease in a sample obtained from the individual after the individual has received one or more doses of the anti-CD33 antibody. In some embodiments, the sample is a cerebrospinal fluid sample. In some embodiments, the one or more biomarkers of Alzheimer's disease are selected from Abeta, Tau, p-Tau, neurofilament light chain, neurogranin, or YKL-40.

In some embodiments which may be combined with any of the preceding aspects or embodiments, the method further comprises determining the presence or absence of one or more variants of APOE, TREM2, CD33, TMEM106b, and/or CLUSTERIN, optionally wherein the one or more variants of APOE comprise an APOE4 variant.

In some embodiments which may be combined with any of the preceding aspects or embodiments, the method further comprises assessing the disease or injury in the individual using one or more imaging assessments performed on the individual after the individual has received one or more doses of the anti-CD33 antibody, wherein the one or more imaging assessments are selected from magnetic resonance imaging (MRI), amyloid-positon emission tomography (PET), or translocator protein (TSPO)-PET.

In some embodiments which may be combined with any of the preceding aspects or embodiments, the method further comprises assessing the disease or injury in the individual using one or more clinical assessments selected from the Clinical Dementia Rating (CDR) Sum of Boxes (CDR-SB) assessment, the Mini-Mental State Examination (MMSE), or the Repeatable Battery for the Assessment of Neuropsychological Status (RBANS).

In some embodiments which may be combined with any of the preceding aspects or embodiments, the method further comprises measuring one or more pharmacokinetics parameters of the anti-CD33 antibody in a sample obtained from the individual after the individual has received one or more doses of the anti-CD33 antibody, optionally wherein the one or more pharmacokinetics parameters are selected from Cₘₐₓ, Tₘₐₓ, AUC₍₀₋ₗₐₛₜ₎, AUC_{(0-inf)}, AUCₜₐᵤ, kₑₗ, t_{1/2}, CL, CLss, V_{z}, or V_{zss}.

In some embodiments which may be combined with any of the preceding aspects or embodiments, the anti-CD33 antibody increases levels of sCD33.

In some embodiments which may be combined with any of the preceding aspects or embodiments, the method further comprises determining the level of sCD33 in the sample obtained from the individual prior to administration of the anti-CD33 antibody or prior to the start of treatment with the anti-CD33 antibody. In some embodiments which may be combined with any of the preceding aspects or embodiments, the method further comprises administering the dose of the anti-CD33 antibody to the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 provides an overview of the Phase 1 study described in Examples 1 and 2. The study includes two parts, Parts 1 and 2. During Part 1, healthy human volunteers (HVs) are administered single doses of placebo or anti-CD33 antibody AB-64.1.2 in doses ranging from 0.05 mg/kg to 60.0 mg/kg (Cohorts A-H). During Part 2, participants with mild-to-moderate Alzheimer's disease (AD) are administered 2 doses of placebo or anti-CD33 antibody AB-64.1.2 at a dose of 15 mg/kg administered 4 weeks apart (i.e., q4w×2). LP, lumbar puncture; MD, multiple doses; PK, pharmacokinetics; SAD, single ascending dose; SD, single dose.

FIGS. 2A-2B provide baseline demographics of participants enrolled in Parts 1 and 2 of the Phase 1 study described in Examples 1 and 2. FIG. 2A provides the baseline demographics of participants enrolled in Part 1 of the study, which included 38 healthy human volunteers treated with anti-CD33 antibody AB-64.1.2 (n=29) or placebo (n=9). FIG. 2B provides the baseline demographics of participants with AD enrolled in Part 2 of the study, which included 12 participants treated with anti-CD33 antibody AB-64.1.2 (n=10) or placebo (n=2). BMI, body mass index; SD, standard deviation.

FIGS. 3A-3B provide safety results for participants enrolled in Parts 1 and 2 of the Phase 1 study described in Examples 1 and 2. The following safety results are provided for healthy human volunteers enrolled in Part 1 of the study (FIG. 3A), and for participants with AD enrolled in Part 2 of the study (FIG. 3B): proportion of participants that experienced one or more treatment-emergent adverse event (“Participants with ≥1 TEAE”); proportion of participants that experienced a TEAE that occurred in 5% or more of all healthy volunteers treated with anti-CD33 antibody AB-64.1.2 in Part 1 of the study (“TEAEs in ≥5% of total AB64.1.2 HVs”); and proportion of participants that experienced one or more serious adverse event (“Participants with ≥1 SAE”). Adverse events (AEs) were coded according to system organ class and preferred terms using the Medical Dictionary for Regulatory Activities Version 24.0. A TEAE was defined as an AE that commenced on or after the time of first study drug administration. If a participant had multiple occurrences of a TEAE, the participant was counted only once in the participant count (n) column for a given system organ class and preferred term. URTI, upper respiratory tract infection.

FIGS. 4A-4D provide pharmacokinetics (PK) results for anti-CD33 antibody AB-64.1.2 in the serum of participants enrolled in Parts 1 and 2 of the Phase 1 study described in Examples 1 and 2. FIG. 4A shows the mean serum concentration of anti-CD33 antibody AB-64.1.2 (y-axis, semi-log; μg/mL+standard deviation [SD]) at the times shown on the x-axis (days) for healthy human volunteers enrolled in each of the cohorts of Part 1 of the study indicated in the legend and by arrows. FIG. 4B provides a summary of mean (SD) serum pharmacokinetics parameters for healthy human volunteers enrolled in each of the indicated cohorts of Part 1 of the study. FIG. 4C shows the mean serum concentration of anti-CD33 antibody AB-64.1.2 (y-axis, semi-log; μg/mL+SD) at the times shown on the x-axis (days) for participants with AD enrolled in Part 2 of the study. FIG. 4D provides a summary of mean (SD) serum pharmacokinetics parameters for participants with AD enrolled in Part 2 of the study. The results shown in FIGS. 4A and 4C are the arithmetic means; the lower limit of quantification was 40.0 ng/mL. AUC_{0-inf}, area under the concentration-time curve from time 0 extrapolated to infinity; AUCₜₐᵤ, area under the drug concentration-time curve over the inter-dosing interval; CL, total body clearance; CLss, steady state clearance; Cₘₐₓ, maximum observed concentration; NA, not available; NC, not calculable; PK, pharmacokinetic; t_{1/2} terminal elimination half-life; tₘₐₓ, time of maximum observed concentration.

FIGS. 5A-5B provide pharmacokinetics results for anti-CD33 antibody AB-64.1.2 in the cerebrospinal fluid (CSF) of participants enrolled in Parts 1 and 2 of the Phase 1 study described in Examples 1 and 2. FIG. 5A shows the mean CSF concentration of anti-CD33 antibody AB-64.1.2 (y-axis, linear; ng/mL+SD) at the times shown on the x-axis (days) for healthy human volunteers enrolled in each of the cohorts of Part 1 of the study indicated in the legend and by arrows. FIG. 5B shows the mean CSF concentration of anti-CD33 antibody AB-64.1.2 (y-axis, linear; ng/ml+SD) at the times shown on the x-axis (days) for participants with AD enrolled in Part 2 of the study.

FIGS. 6A-6B show the levels of CD33 expression on peripheral monocytes of participants enrolled in Parts 1 and 2 of the Phase 1 study described in Examples 1 and 2. FIG. 6A shows the mean percentage change from baseline (+SD) in CD33 expression on peripheral monocytes at the times shown on the x-axis (days) for healthy human volunteers enrolled in each of the cohorts of Part 1 of the study indicated in the legend and by arrows. FIG. 6B shows the mean percentage change from baseline (+SD) in CD33 expression on peripheral monocytes at the times shown on the x-axis (days) for participants with AD enrolled in Part 2 of the study and treated with placebo or anti-CD33 antibody AB-64.1.2 as indicated in the legend and by arrows.

FIGS. 7A-7B show the levels of soluble CD33 (sCD33) in the CSF of participants enrolled in Parts 1 and 2 of the Phase 1 study described in Examples 1 and 2. FIG. 7A shows the mean percentage change from baseline (+SD) in sCD33 levels at the times shown on the x-axis (days) for healthy human volunteers enrolled in each of the cohorts of Part 1 of the study indicated in the legend and by arrows. FIG. 7B shows the mean percentage change from baseline (+SD) in sCD33 levels at the times shown on the x-axis (days) for participants with AD enrolled in Part 2 of the study and treated with placebo or anti-CD33 antibody AB-64.1.2 as indicated in the legend and by arrows.

### DETAILED DESCRIPTION

The present disclosure relates generally to biomarkers of activity and/or therapeutic efficacy of anti-CD33 antibodies for the treatment of a disease or injury in an individual, as well as methods related thereto. As described herein, the methods of the present disclosure meet the need in the art for biomarkers that aid in monitoring and guiding treatment of diseases or injuries with an anti-CD33 antibody.

The present disclosure describes a study of an exemplary anti-CD33 antibody in healthy humans and in individuals with Alzheimer's disease, see, e.g., Examples 1-2. Applicant discovered that administration of an exemplary anti-CD33 antibody to healthy humans and to individuals with Alzheimer's disease resulted in increases in the levels of soluble CD33 protein (sCD33) of at least about 200% over baseline, e.g., in cerebrospinal fluid (see, e.g., Example 2). Applicant further discovered that such increases in the levels of sCD33 were durable over extended periods of time after administration of the anti-CD33 antibody, e.g., up to about 18 days, about 35 days, or more, after administration of the antibody (see, e.g., Example 2). Thus, Applicant recognized that increases in sCD33 levels in an individual (e.g., in blood or cerebrospinal fluid) after administration of an anti-CD33 antibody indicate that the antibody is engaging its target, i.e., CD33 protein, in the individual, and may thus be used to assess the activity and therapeutic efficacy of an anti-CD33 antibody in an individual having a disease or injury.

Accordingly, the present disclosure provides methods for monitoring, assessing, and/or guiding treatment of an individual having a disease or injury with an anti-CD33 antibody by assessing the activity and/or therapeutic efficacy of the anti-CD33 antibody in the individual based, at least in part, on the levels of sCD33. The methods of the disclosure may find use in methods for monitoring, assessing, and/or guiding treatment with anti-CD33 antibody of diseases or injuries including dementia, frontotemporal dementia, Alzheimer's disease, vascular dementia, mixed dementia, and taupathy disease.

All references cited herein, including patents, patent applications and publications, are hereby incorporated by reference in their entirety.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.); PCR 2: A Practical Approach (M. J. MacPherson, B. D. Hames and G. R. Taylor eds. (1995)); Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987; Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. De Vita et al., eds., J.B. Lippincott Company, 1993).

### II. Definitions

As used herein, the terms “treatment”, “treat”, or “treating” refer to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

As used herein, “administering” with respect to a treatment, e.g., an antibody of the disclosure, refers to administration to an individual an effective amount of a treatment, e.g., an antibody of the disclosure, wherein an effective amount refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an effective amount may be considered in the context of administering one or more therapeutic agents, and a single agent may be considered to be given in an effective amount if, in conjunction with one or more other agents, a desirable result may be or is achieved.

An “individual” refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three dimensional configurations of different classes of immunoglobulins are well known and described generally in, for example, Abbas et al., Cellular and Molecular Immunology, 4^{th} ed. (W.B. Saunders Co., 2000).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

An “isolated” antibody, such as an anti-CD33 antibody of the present disclosure, is one that has been identified, separated and/or recovered from a component of its production environment (e.g., naturally or recombinantly). Preferably, the isolated polypeptide is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T cells since at least one component of the antibody's natural environment will not be present. Ordinarily, however, an isolated polypeptide or antibody will be prepared by at least one purification step.

The “variable region” or “variable domain” of an antibody, such as an anti-CD33 antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-CD33 antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

The term “monoclonal antibody” as used herein refers to an antibody, such as an anti-CD33 antibody of the present disclosure, obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against one or more antigenic sites. In some embodiments, a monoclonal antibody of the present disclosure can be a bispecific antibody. In contrast to polyclonal antibody preparations which typically include different antibodies directed against different determinants (epitopes), each monoclonal antibody is directed against a single determinant on the one or more antigenic sites. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by a variety of techniques, including, for example, phage-display technologies (see, e.g., Clackson et al., Nature, 352:624-628 (1991); Marks et al., J. Mol. Biol. 222:581-597 (1992); Sidhu et al., J. Mol. Biol. 338 (2): 299-310 (2004); Lee et al., J. Mol. Biol. 340 (5): 1073-1093 (2004); Fellouse, Proc. Nat'l Acad. Sci. USA 101 (34): 12467-472 (2004); and Lee et al., J. Immunol. Methods 284 (1-2): 119-132 (2004), the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2d ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), yeast presentation technologies (see, e.g., WO2009/036379A2; WO2010105256; WO2012009568, and Xu et al., Protein Eng. Des. Sel., 26 (10): 663-70 (2013), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Nat'l Acad. Sci. USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lonberg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The terms “full-length antibody,” “intact antibody” or “whole antibody” are used interchangeably to refer to an antibody, such as an anti-CD33 antibody of the present disclosure, in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8 (10): 1057-1062 (1995)); single-chain antibody molecules and multispecific antibodies formed from antibody fragments.

Papain digestion of antibodies, such as anti-CD33 antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding. i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of antibodies, such as anti-CD33 antibodies of the present disclosure, comprise a portion of an intact antibody, generally including the antigen binding or variable region of the intact antibody or the F region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Nat'l Acad. Sci. USA 90:6444-48 (1993).

As used herein, a “chimeric antibody” refers to an antibody (immunoglobulin), such as an anti-CD33 antibody of the present disclosure, in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as anti-CD33 antibodies of the present disclosure, are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody, such as an anti-CD33 antibody of the present disclosure, produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad. Sci. USA, 103:3557-3562 (2006) regarding human antibodies generated via a human B-cell hybridoma technology. Alternatively, human antibodies can also be prepared by employing yeast libraries and methods as disclosed in, for example, WO2009/036379A2; WO2010105256; WO2012009568; and Xu et al., Protein Eng. Des. Sel., 26 (10): 663-70 (2013).

The terms “hypervariable region,” “HVR,” or “HV,” when used herein refer to the regions of an antibody-variable domain, such as that of an anti-CD33 antibody of the present disclosure, that are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu in Methods in Molecular Biology 248:1-25 (Lo, ed., Human Press, Totowa, N J, 2003)). Indeed, naturally occurring camelid antibodies consisting of a heavy chain only are functional and stable in the absence of light chain. See, e.g., Hamers-Casterman et al., Nature 363:446-448 (1993) and Sheriff et al., Nature Struct. Biol. 3:733-736 (1996).

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to EU or Kabat et al., supra, for each of these extended-HVR definitions.

“Framework” or “FR” residues are those variable-domain residues other than the HVR residues as herein defined.

The phrase “variable-domain residue-numbering as in EU or Kabat” or “amino-acid-position numbering as in EU or Kabat,” and variations thereof, refers to the numbering system used for heavy-chain variable domains or light-chain variable domains of the compilation of antibodies in EU or Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insertion (residue 52a according to Kabat) after residue 52 of H2 and inserted residues (e.g., residues 82a, 82b, and 82c, etc. according to Kabat) after heavy-chain FR residue 82. The EU or Kabat numbering of residues may be determined for a given antibody by alignment at regions of homology of the sequence of the antibody with a “standard” Kabat numbered sequence.

The EU or Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU or Kabat numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU or Kabat numbering system (e.g., see United States Patent Publication No. 2010-280227).

An “acceptor human framework” as used herein is a framework comprising the amino acid sequence of a VL or VH framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, preferably those changes occur at only three, two, or one of positions 71H, 73H and 78H; for instance, the amino acid residues at those positions may be 71A, 73T and/or 78A. In one embodiment, the VL acceptor human framework is identical in sequence to the VL human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position, e.g., of an anti-CD33 antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-CD33 antibody of the present disclosure, is one with one or more alterations in one or more HVRs thereof that result in an improvement in the affinity of the antibody for the target antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154 (7): 3310-9 (1995); and Hawkins et al, J. Mol. Biol. 226:889-896 (1992).

As used herein, the term “specifically recognizes” or “specifically binds” refers to measurable and reproducible interactions such as attraction or binding between a target and an antibody, such as an anti-CD33 antibody of the present disclosure, that is determinative of the presence of the target in the presence of a heterogeneous population of molecules including biological molecules. For example, an antibody, such as an anti-CD33 antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶M⁻¹, in other instances about 10⁶ M⁻¹ or 10⁷M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a CD33 protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

An “agonist” antibody or an “activating” antibody is an antibody, such as an agonist anti-CD33 antibody of the present disclosure, that induces (e.g., increases) one or more activities or functions of the antigen after the antibody binds the antigen.

A “blocking” antibody, an “antagonist” antibody, or an “inhibitory” antibody is an antibody, such as an anti-CD33 antibody of the present disclosure, that inhibits or reduces (e.g., decreases) antigen binding to one or more ligands after the antibody binds the antigen, and/or that inhibits or reduces (e.g., decreases) one or more activities or functions of the antigen after the antibody binds the antigen. In some embodiments, blocking antibodies, antagonist antibodies, or inhibitory antibodies substantially or completely inhibit antigen binding to one or more ligands and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU or Kabat numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. (see, e.g., M. Daëron, Annu. Rev. Immunol. 15:203-234 (1997)). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. FcRs can also increase the serum half-life of antibodies.

Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants with improved or diminished binding to FcRs. See also, e.g., Shields et al., J. Biol. Chem. 9 (2): 6591-6604 (2001).

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” nucleic acid molecule encoding an antibody, such as an anti-CD33 antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into a viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P (O) S (“thioate”), P(S) S (“dithioate”), (O) NR2 (“amidate”), P (O)R, P (O) OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The terms “active” or “activity” with respect to an agent in an individual, e.g., an anti-CD33 antibody in an individual, refer to the agent engaging with its intended target in the individual. For example, an anti-CD33 antibody is “active” in an individual, or has “activity” in an individual, if upon administration of the anti-CD33 antibody to the individual, the anti-CD33 antibody engages (e.g., binds to) its intended target in the individual, e.g., a CD33 protein. The terms “active” or “activity” with respect to an agent in an individual, e.g., an anti-CD33 antibody in an individual, may also refer to the ability of the agent to have therapeutic efficacy for the treatment of a disease or injury in the individual. For example, an anti-CD33 antibody is “active” in an individual having a disease or injury, or has “activity” in an individual having a disease or injury, if upon administration of the anti-CD33 antibody to the individual, the anti-CD33 antibody alters the natural course of the disease or injury in the individual, decreases the rate of progression of the disease or injury in the individual, ameliorates or palliates the disease or injury in the individual, results in remission of the disease or injury in the individual, improves prognosis of the disease or injury in the individual, and/or mitigates or eliminates one or more symptoms associated with the disease or injury in the individual. The terms “active” or “activity” with respect to an anti-CD33 antibody in an individual may also refer to the ability of the anti-CD33 antibody to modulate, e.g., increase or decrease, one or more activities of CD33 in an individual, e.g., as described in further detail herein. For example, an anti-CD33 antibody is “active” in an individual, or has “activity” in an individual, if upon administration of the anti-CD33 antibody to the individual, the anti-CD33 antibody results in a reduction of cell surface levels of CD33 in the individual, e.g., on one or more cells such as monocytes, dendritic cells, or macrophages. In another example, an anti-CD33 antibody is “active” in an individual, or has “activity” in an individual, if upon administration of the anti-CD33 antibody to the individual, the anti-CD33 antibody results in an increase of sCD33 levels in the individual, e.g., in cerebrospinal fluid and/or blood.

The term “therapeutic efficacy” with respect to an agent in an individual, e.g., an anti-CD33 antibody in an individual, refers to the agent, e.g., an anti-CD33 antibody, altering the natural course of a disease or injury in an individual, decreasing the rate of progression of a disease or injury in an individual, ameliorating or palliating a disease or injury in an individual, resulting in remission of a disease or injury in an individual, improving prognosis of a disease or injury in an individual, and/or mitigating or eliminating one or more symptoms associated with the disease or injury in an individual.

The terms “soluble CD33 protein” or “sCD33” refer to a form of a CD33 protein, e.g., a human CD33 protein, that is not associated or bound to a cell membrane. “Soluble CD33 protein” or “sCD33” encompass full length CD33 proteins, and variants thereof, that are not associated or bound to a cell membrane. “Soluble CD33 protein” or “sCD33” also encompass any fragment of a CD33 protein, or variants thereof, that are not associated or bound to a cell membrane. “Soluble CD33 protein” or “sCD33” also encompass CD33 proteins that are not associated or bound to a cell membrane and that lack one or more domains, such as a transmembrane domain and/or a cytoplasmic domain, for example, as a result of proteolysis of the CD33 protein (e.g., in an endosome in a cell). In some cases, a “soluble CD33 protein” or “sCD33” comprises the IgV domain of a CD33 protein, or a fragment thereof. In some cases, a “soluble CD33 protein” or “sCD33” comprises the IgC2 domain of a CD33 protein, or a fragment thereof. In some cases, a “soluble CD33 protein” or “sCD33” comprises the IgV domain of a CD33 protein, or a fragment thereof, and the IgC2 domain of a CD33 protein, or a fragment thereof.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural referents unless the context clearly indicates otherwise. For example, reference to an “antibody” is a reference to from one to many antibodies, such as molar amounts, and includes equivalents thereof known to those skilled in the art, and so forth.

It is understood that aspects and embodiments of the present disclosure described herein include “comprising,” “consisting,” and “consisting essentially of” aspects and embodiments.

### III. Methods of the Disclosure

As disclosed herein, increases in the levels of soluble CD33 protein (sCD33) in an individual (e.g., in blood or cerebrospinal fluid) after administration of an anti-CD33 antibody may be used to assess the activity and therapeutic efficacy of the anti-CD33 antibody in an individual having a disease or injury.

Accordingly, in some aspects, provided herein are methods for assessing the activity or therapeutic efficacy of an anti-CD33 antibody in an individual having a disease or injury based, at least in part, on the levels of sCD33 in a sample from the individual. In other aspects, provided herein are methods for monitoring, guiding, or adjusting treatment with an anti-CD33 antibody in an individual having a disease or injury based, at least in part, on the levels of sCD33 in a sample from the individual. In other aspects, provided herein are methods for providing information on the activity or therapeutic efficacy of an anti-CD33 antibody in an individual having a disease or injury based, at least in part, on the levels of sCD33 in a sample from the individual. In yet other aspects, also provided herein are methods for treating and/or delaying progression of a disease or injury in an individual by administering an anti-CD33 antibody.

In some embodiments of any of the methods provided herein, an increase in the levels of sCD33 in a sample from an individual after treatment with an anti-CD33 antibody indicates that the anti-CD33 antibody is active in the individual and/or has therapeutic efficacy in the individual for the treatment of a disease or injury, e.g., as described in further detail below. In other embodiments, an increase in the levels of sCD33 in a sample from an individual after treatment with an anti-CD33 antibody indicates that treatment with the anti-CD33 antibody should continue, e.g., as described in further detail below. In other embodiments, absence of an increase in the levels of sCD33 in a sample from an individual after treatment with an anti-CD33 antibody indicates the need to adjust, e.g., increase, the dose or frequency of anti-CD33 antibody being administered to the individual, e.g., as described in further detail below.

In some embodiments, the disease or injury is a disease or injury provided herein. In some embodiments, the disease or injury is dementia, frontotemporal dementia, Alzheimer's disease, vascular dementia, mixed dementia, and taupathy disease. In some embodiments, the disease or injury is Alzheimer's disease. In some embodiments, the disease or injury is mild to moderate Alzheimer's disease.

### A. Assessing, Monitoring, Guiding or Adjusting Anti-CD33 Antibody Treatment Based on Soluble CD33 Levels

In some embodiments, the methods of the disclosure comprise assessing the activity and/or therapeutic efficacy of an anti-CD33 antibody in an individual having a disease or injury based, at least in part, on the level of soluble CD33 protein (sCD33) in one or more samples obtained from the individual. In some embodiments, an increase in the level of sCD33 in a sample obtained from the individual after the individual has received a dose of anti-CD33 antibody indicates that the anti-CD33 antibody is active in the individual, that the anti-CD33 antibody has therapeutic efficacy in the individual, and/or that treatment with the anti-CD33 antibody should continue. In some embodiments, absence of an increase in the level of sCD33 in a sample obtained from the individual after the individual has received a dose of anti-CD33 antibody indicates that treatment with the anti-CD33 antibody should continue, e.g., at increased doses and/or frequency.

In some embodiments, an increase in the level of sCD33 in a sample obtained from the individual after the individual has received a dose of anti-CD33 antibody is determined as compared to the level of sCD33 in a sample obtained from the individual prior to administration of the anti-CD33 antibody, e.g., as described in further detail in section (i) below. In some embodiments, an increase in the level of sCD33 in a sample obtained from the individual after the individual has received a dose of anti-CD33 antibody is determined as compared to the level of sCD33 in a sample obtained from a corresponding individual not treated with anti-CD33 antibody, e.g., as described in further detail in section (ii) below.

(i) Assessment of sCD33 Levels Compared to Baseline

In some embodiments, administration of an anti-CD33 antibody, such as an antibody described herein, to an individual, e.g., according to a treatment regimen described herein, results in an increase in the levels of sCD33 in a sample obtained from the individual after administration of a dose of the antibody, as compared to the level of sCD33 in a sample from the individual obtained prior to administration of the anti-CD33 antibody.

In some embodiments, an increase in the levels of sCD33 in a sample obtained from the individual after administration of a dose of an anti-CD33 antibody, as compared to the level of sCD33 in a sample from the individual obtained prior to administration of the anti-CD33 antibody, indicates that the anti-CD33 antibody is active in the individual (e.g., that the antibody is engaging its target, i.e., a CD33 protein, in the individual, and/or that the antibody has therapeutic efficacy in the individual). In other embodiments, an increase in the levels of sCD33 in a sample obtained from the individual after administration of a dose of an anti-CD33 antibody, as compared to the level of sCD33 in a sample from the individual obtained prior to administration of the anti-CD33 antibody, indicates that the anti-CD33 antibody has therapeutic efficacy in the individual, e.g., for treating and/or delaying progression of a disease or injury in the individual. In other embodiments, an increase in the levels of sCD33 in a sample obtained from the individual after administration of a dose of an anti-CD33 antibody, as compared to the level of sCD33 in a sample from the individual obtained prior to administration of the anti-CD33 antibody, indicates that treatment with the anti-CD33 antibody should continue (e.g., at the same dose, at a decreased dose, or at an increased dose; and/or at the same frequency, less frequently, or more frequently). In other embodiments, absence of an increase in the levels of sCD33 in a sample obtained from the individual after administration of a dose of an anti-CD33 antibody, as compared to the level of sCD33 in a sample from the individual obtained prior to administration of the anti-CD33 antibody, indicates that treatment with the anti-CD33 antibody should be adjusted or modified, e.g., by increasing the dose or dosing frequency of the anti-CD33 antibody.

In some embodiments, an increase in the levels of sCD33 in a sample obtained from the individual after administration of a dose of the antibody comprises an increase of any of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, at least about 600%, at least about 610%, at least about 620%, at least about 630%, at least about 640%, at least about 650%, at least about 660%, at least about 670%, at least about 680%, at least about 690%, at least about 700%, at least about 710%, at least about 720%, at least about 730%, at least about 740%, at least about 750%, at least about 760%, at least about 770%, at least about 780%, at least about 790%, at least about 800%, at least about 810%, at least about 820%, at least about 830%, at least about 840%, at least about 850%, at least about 860%, at least about 870%, at least about 880%, at least about 890%, at least about 900%, at least about 910%, at least about 920%, at least about 930%, at least about 940%, at least about 950%, at least about 960%, at least about 970%, at least about 980%, at least about 990%, at least about 1000%, or more, as compared to the level of sCD33 in a sample from the individual obtained prior to administration of the anti-CD33 antibody.

In some embodiments, an increase in the levels of sCD33 in a sample obtained from the individual after administration of a dose of the antibody comprises an increase of any of at least about 200%, at least about 400%, at least about 500%, at least about 600%, at least about 700%, or more, as compared to the level of sCD33 in a sample from the individual obtained prior to administration of the anti-CD33 antibody.

In some embodiments, the sample obtained from the individual after administration of a dose of the anti-CD33 antibody is a blood sample (e.g., a whole blood, plasma or serum sample), and the sample from the individual obtained prior to administration of the anti-CD33 antibody is a blood sample (e.g., a whole blood, plasma or serum sample). In some embodiments, the sample obtained from the individual after administration of a dose of the anti-CD33 antibody is a cerebrospinal fluid sample, and the sample from the individual obtained prior to administration of the anti-CD33 antibody is a cerebrospinal fluid sample. In some embodiments, the sample obtained from the individual after administration of a dose of the anti-CD33 antibody is a whole blood sample, and the sample from the individual obtained prior to administration of the anti-CD33 antibody is a whole blood sample. In some embodiments, the sample obtained from the individual after administration of a dose of the anti-CD33 antibody is a plasma sample, and the sample from the individual obtained prior to administration of the anti-CD33 antibody is a plasma sample. In some embodiments, the sample obtained from the individual after administration of a dose of the anti-CD33 antibody is a serum sample, and the sample from the individual obtained prior to administration of the anti-CD33 antibody is a serum sample.

In some embodiments, the sample obtained from the individual after administration of a dose of the anti-CD33 antibody is obtained from the individual any of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 124 days, about 125 days, about 126 days, about 127 days, about 128 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, or more, after the individual has received a dose of the anti-CD33 antibody.

In some embodiments, the sample from the individual obtained prior to administration of the anti-CD33 antibody is obtained within any of about 10 weeks, about 9 weeks, about 8 weeks, about 7 weeks, about 6 weeks, about 5 weeks, about 4 weeks, about 3 weeks, about 2 weeks, about 1 week, or less, prior to administration of the anti-CD33 antibody (e.g., prior to administration of a first dose of an anti-CD33 antibody to the individual; or prior to administration of a dose of an anti-CD33 antibody to the individual). In some embodiments, the sample from the individual obtained prior to administration of the anti-CD33 antibody is obtained within any of about 70 days, about 69 days, about 68 days, about 67 days, about 66 days, about 65 days, about 64 days, about 63 days, about 62 days, about 61 days, about 60 days, about 59 days, about 58 days, about 57 days, about 56 days, about 55 days, about 54 days, about 53 days, about 52 days, about 51 days, about 50 days, about 49 days, about 48 days, about 47 days, about 46 days, about 45 days, about 44 days, about 43 days, about 42 days, about 41 days, about 40 days, about 39 days, about 38 days, about 37 days, about 36 days, about 35 days, about 34 days, about 33 days, about 32 days, about 31 days, about 30 days, about 29 days, about 28 days, about 27 days, about 26 days, about 25 days, about 24 days, about 23 days, about 22 days, about 21 days, about 20 days, about 19 days, about 18 days, about 17 days, about 16 days, about 15 days, about 14 days, about 13 days, about 12 days, about 11 days, about 10 days, about 9 days, about 8 days, about 7 days, about 6 days, about 5 days, about 4 days, about 3 days, about 2 days, about 1 day, or less, prior to administration of the anti-CD33 antibody (e.g., prior to administration of a first dose of an anti-CD33 antibody to the individual; or prior to administration of a dose of an anti-CD33 antibody to the individual).

(ii) Assessment of sCD33 Levels Compared to an Untreated Individual

In some embodiments, administration of an anti-CD33 antibody, such as antibody described herein, to an individual, e.g., according to a treatment regimen described herein, results in an increase in the levels of sCD33 in a sample obtained from the individual after administration of a dose of the antibody, as compared to the level of sCD33 in a sample obtained from a corresponding individual not administered or treated with the anti-CD33 antibody.

In some embodiments, the corresponding individual is an individual (e.g., a human individual) having the same disease or injury as the individual (e.g., a human individual) treated with the anti-CD33 antibody. For example, in some embodiments, the individual (e.g., a human individual) treated with the anti-CD33 antibody has Alzheimer's disease, and the corresponding individual (e.g., a human individual) also has Alzheimer's disease. In some embodiments, the corresponding individual is an individual (e.g., a human individual) having the same disease with matched or similar disease characteristics (e.g., disease type, stage, severity, and the like), as the individual (e.g., a human individual) treated with the anti-CD33 antibody. For example, in some embodiments, the corresponding individual is an individual (e.g., a human individual) having Alzheimer's disease with matched or similar characteristics (e.g., one or more of: clinical diagnosis [e.g., a clinical diagnosis of probable Alzheimer's disease dementia based on National Institute on Aging Alzheimer's Association criteria]; Mini-Mental State Examination score prior to treatment [MMSE; e.g., an MMSE score of between about 16 and about 28 points]; Clinical Dementia Rating-Global Score prior to treatment [CDR-GS; e.g., a CDR-GS score of 0.5, 1.0, or 2.0]; a positive amyloid-PET scan by qualitative read; taking a stable dose of cholinesterase inhibitor and/or memantine therapy for Alzheimer's disease prior to treatment; absence of two copies of the minor allele rs12459419T; absence of, or absence of a history of, central nervous system or systemic autoimmune disorders including, but not limited to, rheumatoid arthritis, multiple sclerosis, lupus erythematosus, anti-phospholipid antibody syndrome, or Behçet disease; absence of dementia due to a condition other than Alzheimer's disease, including, but not limited to, Frontotemporal Dementia, Parkinson's disease, dementia with Lewy bodies, Huntington disease, or vascular dementia; absence of, or absence of history of, clinically evident vascular disease potentially affecting the brain [e.g., clinically significant carotid, vertebral stenosis or plaque, aortic aneurysm, intracranial aneurysm, cerebral hemorrhage, arteriovenous malformation] that has the potential to affect cognitive function; absence of, or absence of history of, stroke within the past 2 years; absence of history of transient ischemic attack within the last 12 months; absence of history of severe, clinically significant [e.g., persistent neurologic deficit or structural brain damage] central nervous system trauma [e.g., cerebral contusion]; and/or absence of MRI evidence of more than two lacunar infarcts, any territorial infarct >1 cm³, or significant FLAIR hyperintense lesions in the cerebral white matter that may contribute to cognitive dysfunction) as the individual (e.g., a human individual) having Alzheimer's disease and treated with the anti-CD33 antibody. In another example, the individual (e.g., a human individual) treated with the anti-CD33 antibody has mild to moderate Alzheimer's disease, and the corresponding individual (e.g., a human individual) also has mild to moderate Alzheimer's disease. In some embodiments, the corresponding individual is an individual (e.g., a human individual) having matched or similar demographics characteristics (e.g., one or more of: sex [i.e., male or female], age, race [e.g., white, Asian, Black, African American, etc.], ethnicity [e.g., Hispanic, or Latino], weight, and/or body mass index), as the individual (e.g., a human individual) treated with the anti-CD33 antibody. In some embodiments, the corresponding individual is administered a placebo, such as normal saline, e.g., using the same method of administration as the anti-CD33 antibody (e.g., by intravenous administration).

In some embodiments, an increase in the levels of sCD33 in a sample obtained from an individual after administration of a dose of an anti-CD33 antibody, as compared to the level of sCD33 in a sample from a corresponding individual not administered the anti-CD33 antibody, indicates that the anti-CD33 antibody is active in the individual (e.g., that the antibody is engaging its target, i.e., a CD33 protein, in the individual, and/or that the antibody has therapeutic efficacy in the individual). In other embodiments, an increase in the levels of sCD33 in a sample obtained from an individual after administration of a dose of an anti-CD33 antibody, as compared to the level of sCD33 in a sample from a corresponding individual not administered the anti-CD33 antibody, indicates that the anti-CD33 antibody has therapeutic efficacy in the individual, e.g., for treating and/or delaying progression of a disease or injury in the individual. In other embodiments, an increase in the levels of sCD33 in a sample obtained from an individual after administration of a dose of the antibody, as compared to the level of sCD33 in a sample from a corresponding individual not administered the anti-CD33 antibody, indicates that treatment with the anti-CD33 antibody should continue (e.g., at the same dose, at a decreased dose, or at an increased dose; and/or at the same frequency, less frequently, or more frequently). In other embodiments, absence of an increase in the levels of sCD33 in a sample obtained from an individual after administration of a dose of an anti-CD33 antibody, as compared to the level of sCD33 in a sample from a corresponding individual not administered the anti-CD33 antibody, indicates that treatment with the anti-CD33 antibody should be adjusted or modified, e.g., by increasing the dose or dosing frequency of the anti-CD33 antibody.

In some embodiments, an increase in the levels of sCD33 in a sample obtained from an individual after administration of a dose of an anti-CD33 antibody comprises an increase of any of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, at least about 600%, at least about 610%, at least about 620%, at least about 630%, at least about 640%, at least about 650%, at least about 660%, at least about 670%, at least about 680%, at least about 690%, at least about 700%, at least about 710%, at least about 720%, at least about 730%, at least about 740%, at least about 750%, at least about 760%, at least about 770%, at least about 780%, at least about 790%, at least about 800%, at least about 810%, at least about 820%, at least about 830%, at least about 840%, at least about 850%, at least about 860%, at least about 870%, at least about 880%, at least about 890%, at least about 900%, at least about 910%, at least about 920%, at least about 930%, at least about 940%, at least about 950%, at least about 960%, at least about 970%, at least about 980%, at least about 990%, at least about 1000%, or more, as compared to the level of sCD33 in a sample from a corresponding individual not administered the anti-CD33 antibody.

In some embodiments, an increase in the levels of sCD33 in a sample obtained from an individual after administration of a dose of an anti-CD33 antibody comprises an increase of any of at least about 200%, at least about 400%, at least about 500%, at least about 600%, at least about 700%, or more, as compared to the level of sCD33 in a sample from a corresponding individual not administered the anti-CD33 antibody.

In some embodiments, the sample obtained from the individual after administration of a dose of the anti-CD33 antibody is a blood sample (e.g., a whole blood, plasma or serum sample), and the sample obtained from the corresponding individual not administered the anti-CD33 antibody is a blood sample (e.g., a whole blood, plasma or serum sample). In some embodiments, the sample obtained from the individual after administration of a dose of the anti-CD33 antibody is a cerebrospinal fluid sample, and the sample obtained from the corresponding individual not administered the anti-CD33 antibody is a cerebrospinal fluid sample. In some embodiments, the sample obtained from the individual after administration of a dose of the anti-CD33 antibody is a plasma sample, and the sample obtained from the corresponding individual not administered the anti-CD33 antibody is a plasma sample. In some embodiments, the sample obtained from the individual after administration of a dose of the anti-CD33 antibody is a whole blood sample, and the sample obtained from the corresponding individual not administered the anti-CD33 antibody is a whole blood sample. In some embodiments, the sample obtained from the individual after administration of a dose of the anti-CD33 antibody is a serum sample, and the sample obtained from the corresponding individual not administered the anti-CD33 antibody is a serum sample.

In some embodiments, the sample obtained from the individual after administration of a dose of the anti-CD33 antibody is obtained from the individual any of about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 124 days, about 125 days, about 126 days, about 127 days, about 128 days, about 129 days, about 130 days, about 131 days, about 132 days, about 133 days, about 134 days, about 135 days, about 136 days, about 137 days, about 138 days, about 139 days, about 140 days, about 141 days, about 142 days, about 143 days, about 144 days, about 145 days, about 146 days, about 147 days, about 148 days, about 149 days, about 150 days, about 151 days, about 152 days, about 153 days, about 154 days, about 155 days, about 156 days, about 157 days, about 158 days, about 159 days, about 160 days, about 161 days, about 162 days, about 163 days, about 164 days, about 165 days, about 166 days, about 167 days, about 168 days, about 169 days, about 170 days, about 171 days, about 172 days, about 173 days, about 174 days, about 175 days, about 176 days, about 177 days, about 178 days, about 179 days, about 180 days, about 181 days, about 182 days, about 183 days, about 184 days, about 185 days, about 186 days, about 187 days, about 188 days, about 189 days, about 190 days, about 191 days, about 192 days, about 193 days, about 194 days, about 195 days, about 196 days, about 197 days, about 198 days, about 199 days, about 200 days, or more, after the individual has received a dose of the anti-CD33 antibody.

(iii) Methods for Determining sCD33 Levels

Any suitable methods known in the art may be used to determine the levels of sCD33 in a sample. For example, sCD33 levels in a sample (e.g., a cerebrospinal fluid sample, or a blood sample such as a whole blood, serum or plasma sample) may be determined using immunoblots (e.g., Western blots), mass spectrometry, flow cytometry, an immunoassay (e.g., a SIMOA assay from Quanterix, see, e.g., the website: www.quanterix.com/simoa-technology/), a proximity extension assay (e.g., an assay from Olink, see, e.g., the website: www.olink.com/our-platform/our-pea-technology), an electrochemiluminescence-based assay (e.g., an assay from Meso Scale Diagnostics, see, e.g., the website: www.mesoscale.com/en/technical_resources/our_technology/ecl), and/or aptamer-based methods such as SOMASCAN assay (see. e.g., Candia et al. (2017) Sci Rep 7, 14248).

In some embodiments, sCD33 levels in a sample (e.g., a cerebrospinal fluid sample, or a blood sample such as a whole blood, serum or plasma sample) are determined using a mass spectrometry-based method. For example, sCD33 levels in a sample (e.g., a cerebrospinal fluid sample, or a blood sample such as a whole blood, serum or plasma sample) may be determined using a Quantitative Liquid Chromatography Multiple-Reaction Monitoring Mass Spectrometry (LCMRM/MS) assay. Determination of sCD33 levels using a Quantitative Liquid Chromatography Multiple-Reaction Monitoring Mass Spectrometry (LCMRM/MS) assay may comprise one or more, or all, of the following steps, as described in Example 2 herein: (a) preparation of calibration standards (e.g., calibration standards may be prepared at levels, e.g., eight levels, covering the assay range by spiking [i.e., adding] CD33 protein, e.g., recombinant CD33 protein, in surrogate matrix, e.g., surrogate matrix for the sample from the individual treated with anti-CD33 antibody [e.g., a surrogate matrix for cerebrospinal fluid, whole blood, serum or plasma, such as a suitable buffer solution]); (b) preparation of quality control (QC) samples (e.g., QC samples may be prepared at levels, e.g., three levels, covering the assay range by spiking CD33 protein, e.g., recombinant CD33 protein, in pooled samples from reference individuals [e.g., in pooled samples of cerebrospinal fluid, whole blood, serum or plasma from healthy individuals]), and/or QC samples at endogenous levels (non-spiked QC samples, e.g., non-spiked QC samples may be prepared from pooled samples from reference individuals [e.g., pooled samples of cerebrospinal fluid, whole blood, serum or plasma from healthy individuals] not spiked with CD33 protein), and/or blank samples; (c) providing samples from one or more individuals treated with anti-CD33 antibody, e.g., cerebrospinal fluid samples, or blood samples such as a whole blood, serum or plasma samples; (d) digesting, e.g., with trypsin or another suitable enzyme, the calibration standards, QC samples, non-spiked QC samples, blank samples, and/or samples from one or more individuals treated with anti-CD33 antibody; (e) spiking (i.e., adding to) the calibration standards, QC samples, non-spiked QC samples, blank samples, and/or samples from one or more individuals treated with anti-CD33 antibody a Stable Isotope Labeled (SIL) internal standard peptide for sCD33 (e.g., FAGAGVTTER [SEQ ID NO: 27]); (f) desalting the calibration standards, QC samples, non-spiked QC samples, blank samples, and/or samples from one or more individuals treated with anti-CD33 antibody using Solid-Phase Extraction (SPE) sorbent matrix; (g) analyzing the calibration standards, QC samples, non-spiked QC samples, blank samples, and/or samples from one or more individuals treated with anti-CD33 antibody using an LC-MRM/MS assay, wherein transitions from endogenous target peptide (FAGAGVTTER [SEQ ID NO: 27]) and its SIL counterpart are monitored; (h) determining the concentration of sCD33 in the samples from one or more individuals treated with anti-CD33 antibody by calculating the ratio of endogenous target peptide and SIL peptide and back-calculating the value onto a calibration curve.

### B. Treatment Adjustments or Modifications

In some embodiments of any of the methods provided herein, the methods comprise determining that an anti-CD33 antibody is active in an individual and/or has therapeutic efficacy in the individual, e.g., according to any of the methods described herein, for example in sections (i)-(ii) above. In some embodiments of any of the methods provided herein, the methods comprise determining that treatment with the anti-CD33 antibody should continue (e.g., at the same dose, at a decreased dose, or at an increased dose; and/or at the same frequency, less frequently, or more frequently), e.g., according to any of the methods described herein, for example in sections (i)-(ii) above. In some embodiments of any of the methods provided herein, the methods comprise determining that treatment with the anti-CD33 antibody should be adjusted or modified (e.g., by increasing the dose or dosing frequency of the anti-CD33 antibody), e.g., according to any of the methods described herein, for example in sections (i)-(ii) above.

In some embodiments, the methods further comprise administering one or more additional doses of an anti-CD33 antibody to the individual responsive to determining that the anti-CD33 antibody is active in the individual and/or has therapeutic efficacy in the individual, e.g., according to any of the methods described herein, for example in sections (i)-(ii) above. In some embodiments, the methods further comprise administering one or more additional doses of an anti-CD33 antibody to the individual responsive to determining that treatment with the anti-CD33 antibody should continue (e.g., at the same dose and/or dosing frequency), e.g., according to any of the methods described herein, for example in sections (i)-(ii) above. In some such embodiments, the methods comprise administering any of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, or more, additional doses of the anti-CD33 antibody to the individual. In some embodiments, the one or more additional doses of the anti-CD33 antibody are administered at the same dose as a prior dose of anti-CD33 antibody administered to the individual. In some embodiments, the one or more additional doses of the anti-CD33 antibody are administered at the same dosing frequency previously administered to the individual. In other embodiments, the methods comprise continuing treatment with the anti-CD33 antibody for at least about 1 week, at least about 2 weeks, at least about 3 weeks, at least about 4 weeks, at least about 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, at least 60 months, at least 61 months, at least 62 months, at least 63 months, at least 64 months, at least 65 months, at least 66 months, at least 67 months, at least 68 months, at least 69 months, at least 70 months, at least 71 months, at least 72 months, at least 73 months, at least 74 months, at least 75 months, at least 76 months, at least 77 months, at least 78 months, at least 79 months, at least 80 months, at least 81 months, at least 82 months, at least 83 months, at least 84 months, at least 85 months, at least 86 months, at least 87 months, at least 88 months, at least 89 months, at least 90 months, at least 91 months, at least 92 months, at least 93 months, at least 94 months, at least 95 months, at least 96 months, at least 97 months, at least 98 months, at least 99 months, at least 100 months, at least 101 months, at least 102 months, at least 103 months, at least 104 months, at least 105 months, at least 106 months, at least 107 months, at least 108 months, at least 109 months, at least 110 months, at least 111 months, at least 112 months, at least 113 months, at least 114 months, at least 115 months, at least 116 months, at least 117 months, at least 118 months, at least 119 months, at least 120 months, or more. In some embodiments, treatment with the anti-CD33 antibody is continued at the same dose as a prior dose of anti-CD33 antibody administered to the individual. In some embodiments, treatment with the anti-CD33 antibody is continued at the same dosing frequency of anti-CD33 antibody previously administered to the individual.

In some embodiments, the methods further comprise administering one or more additional doses of an anti-CD33 antibody to the individual responsive to determining that treatment with the anti-CD33 antibody should continue (e.g., at a decreased dose, or at an increased dose; and/or less frequently, or more frequently), e.g., according to any of the methods described herein, for example in sections (i)-(ii) above. In some embodiments, the methods further comprise administering one or more additional doses of an anti-CD33 antibody to the individual responsive to determining that treatment with the anti-CD33 antibody should be adjusted or modified (e.g., by increasing the dose or dosing frequency of the anti-CD33 antibody), e.g., according to any of the methods described herein, for example in sections (i)-(ii) above. In some such embodiments, the methods comprise adjusting or modifying the treatment of the individual with the anti-CD33 antibody, e.g., by increasing or decreasing the dose of anti-CD33 antibody administered to the individual, and/or by increasing or decreasing the frequency of administration of anti-CD33 antibody to the individual. In some embodiments, the methods comprise increasing the dose of anti-CD33 antibody administered to the individual by any of at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 225-fold, at least about 250-fold, at least about 275-fold, at least about 300-fold, at least about 325-fold, at least about 350-fold, at least about 375-fold, at least about 400-fold, at least about 425-fold, at least about 450-fold, at least about 475-fold, at least about 500-fold, at least about 525-fold, at least about 550-fold, at least about 575-fold, at least about 600-fold, at least about 625-fold, at least about 650-fold, at least about 675-fold, at least about 700-fold, at least about 725-fold, at least about 750-fold, at least about 775-fold, at least about 800-fold, at least about 825-fold, at least about 850-fold, at least about 875-fold, at least about 900-fold, at least about 925-fold, at least about 950-fold, at least about 975-fold, at least about 1000-fold, at least about 1025-fold, at least about 1050-fold, at least about 1075-fold, at least about 1100-fold, at least about 1125-fold, at least about 1150-fold, at least about 1175-fold, at least about 1200-fold, or more, as compared to a prior dose of anti-CD33 antibody administered to the individual. In some embodiments, the methods comprise increasing the frequency of administration of the anti-CD33 antibody to the individual, e.g., to any of once every eight weeks or more frequently (e.g., about once every 56 days or more frequently), once every seven weeks or more frequently (e.g., about once every 49 days or more frequently), once every six weeks or more frequently (e.g., about once every 42 days or more frequently), once every five weeks or more frequently (e.g., about once every 35 days or more frequently), once every four weeks or more frequently (e.g., about once every 28 days or more frequently), once every three weeks or more frequently (e.g., about once every 21 days or more frequently), once every two weeks or more frequently (e.g., about once every 14 days or more frequently), or once every week (e.g., about once every 7 days or more frequently). In some embodiments, the methods comprise decreasing the dose of anti-CD33 antibody administered to the individual by any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to a prior dose of anti-CD33 antibody administered to the individual. In some embodiments, the methods comprise decreasing the frequency of administration of the anti-CD33 antibody to the individual, e.g., to any of once every eight weeks or less frequently (e.g., about once every 56 days or less frequently), once every seven weeks or less frequently (e.g., about once every 49 days or less frequently), once every six weeks or less frequently (e.g., about once every 42 days or less frequently), once every five weeks or less frequently (e.g., about once every 35 days or less frequently), once every four weeks or less frequently (e.g., about once every 28 days or more frequently), once every three weeks or less frequently (e.g., about once every 21 days or less frequently), once every two weeks or less frequently (e.g., about once every 14 days or less frequently), or once every week (e.g., about once every 7 days or less frequently). In some embodiments, the methods comprise administering any of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, or more, additional doses of the anti-CD33 antibody to the individual, e.g., at an increased dose, at a decreased dose, at an increased frequency, and/or at a decreased frequency, e.g., as described above. In some embodiments, the methods comprise continuing treatment of the individual with the anti-CD33 antibody for at least about 1 week, at least about 2 weeks, at least about 3 weeks, at least about 4 weeks, at least about 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, at least 60 months, at least 61 months, at least 62 months, at least 63 months, at least 64 months, at least 65 months, at least 66 months, at least 67 months, at least 68 months, at least 69 months, at least 70 months, at least 71 months, at least 72 months, at least 73 months, at least 74 months, at least 75 months, at least 76 months, at least 77 months, at least 78 months, at least 79 months, at least 80 months, at least 81 months, at least 82 months, at least 83 months, at least 84 months, at least 85 months, at least 86 months, at least 87 months, at least 88 months, at least 89 months, at least 90 months, at least 91 months, at least 92 months, at least 93 months, at least 94 months, at least 95 months, at least 96 months, at least 97 months, at least 98 months, at least 99 months, at least 100 months, at least 101 months, at least 102 months, at least 103 months, at least 104 months, at least 105 months, at least 106 months, at least 107 months, at least 108 months, at least 109 months, at least 110 months, at least 111 months, at least 112 months, at least 113 months, at least 114 months, at least 115 months, at least 116 months, at least 117 months, at least 118 months, at least 119 months, at least 120 months, or more, e.g., at an increased dose, at a decreased dose, at an increased frequency, and/or at a decreased frequency, e.g., as described above.

### C. Providing Information, a Recommendation, or Reporting

In some embodiments, the methods of the disclosure comprise providing information on the levels of soluble CD33 protein (sCD33) in a sample from an individual, e.g., determined according to any suitable method, such as any method known in the art or described herein. In some embodiments, the methods of the disclosure comprise providing information on the levels of sCD33 in a sample from an individual obtained before an individual has received a dose of an anti-CD33 antibody. In some embodiments, the methods of the disclosure comprise providing information on the levels of sCD33 in a sample from an individual obtained after an individual has received a dose of an anti-CD33 antibody. In some embodiments, the methods of the disclosure comprise providing information on the levels of sCD33 in a sample from an individual obtained after the individual has received more than one dose of an anti-CD33 antibody. In some embodiments, the methods of the disclosure comprise providing information indicating that an increase in the levels of sCD33 is present in a sample obtained from the individual after administration of a dose of an anti-CD33 antibody, as compared to the level of sCD33 in a sample from the individual obtained prior to administration of the anti-CD33 antibody. In some embodiments, the methods of the disclosure comprise providing information indicating that an increase in the levels of sCD33 is present in a sample obtained from the individual after administration of a dose of an anti-CD33 antibody, as compared to the level of sCD33 in a sample from a corresponding individual not treated with the anti-CD33 antibody.

In other embodiments, the methods of the disclosure comprise providing information indicating that an anti-CD33 antibody is (or has been determined to be) active in an individual, and/or has (or has been determined to have) therapeutic efficacy in the individual, e.g., according to any of the methods described herein, for example in Sections A-B above. In some embodiments, such information is based, at least in part, on the level of soluble CD33 protein (sCD33) in one or more samples obtained from the individual, e.g., after the individual has received one or more doses of an anti-CD33 antibody. In some embodiments, such information is based, at least in part, on the presence of an increase in the levels of sCD33 in a sample obtained from the individual after administration of a dose of an anti-CD33 antibody, as compared to the level of sCD33 in a sample from the individual obtained prior to administration of the anti-CD33 antibody. In some embodiments, such information is based, at least in part, on the presence of an increase in the levels of sCD33 in a sample obtained from the individual after administration of a dose of an anti-CD33 antibody, as compared to the level of sCD33 in a sample from a corresponding individual not treated with the anti-CD33 antibody. In some embodiments, such information may comprise a recommendation to continue treatment of the individual with the anti-CD33 antibody, e.g., based, at least in part, on determining that the anti-CD33 antibody is active in an individual, and/or has therapeutic efficacy in the individual. In some embodiments, such information may comprise a recommendation to administer one or more additional doses of the anti-CD33 antibody, e.g., based, at least in part, on determining that the anti-CD33 antibody is active in an individual, and/or has therapeutic efficacy in the individual. In some embodiments, such information may comprise a recommendation to continue treatment with the anti-CD33 antibody at the same dose and/or at the same frequency previously administered to the individual, e.g., based, at least in part, on determining that the anti-CD33 antibody is active in an individual, and/or has therapeutic efficacy in the individual. In some embodiments, such information may comprise a recommendation to administer one or more additional doses of the anti-CD33 antibody at the same dose and/or at the same frequency previously administered to the individual, e.g., based, at least in part, on determining that the anti-CD33 antibody is active in an individual, and/or has therapeutic efficacy in the individual. In some embodiments, said recommendation indicates that at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, or more, additional doses of the anti-CD33 antibody should be administered to the individual. In some embodiments, the recommendation indicates that the additional doses of anti-CD33 antibody should be administered at the same dose and/or frequency previously administered to the individual. In some embodiments, said recommendation indicates that the individual should continue to receive treatment with the anti-CD33 antibody for at least about 1 week, at least about 2 weeks, at least about 3 weeks, at least about 4 weeks, at least about 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, at least 60 months, at least 61 months, at least 62 months, at least 63 months, at least 64 months, at least 65 months, at least 66 months, at least 67 months, at least 68 months, at least 69 months, at least 70 months, at least 71 months, at least 72 months, at least 73 months, at least 74 months, at least 75 months, at least 76 months, at least 77 months, at least 78 months, at least 79 months, at least 80 months, at least 81 months, at least 82 months, at least 83 months, at least 84 months, at least 85 months, at least 86 months, at least 87 months, at least 88 months, at least 89 months, at least 90 months, at least 91 months, at least 92 months, at least 93 months, at least 94 months, at least 95 months, at least 96 months, at least 97 months, at least 98 months, at least 99 months, at least 100 months, at least 101 months, at least 102 months, at least 103 months, at least 104 months, at least 105 months, at least 106 months, at least 107 months, at least 108 months, at least 109 months, at least 110 months, at least 111 months, at least 112 months, at least 113 months, at least 114 months, at least 115 months, at least 116 months, at least 117 months, at least 118 months, at least 119 months, at least 120 months, or more. In some embodiments, the recommendation indicates that the continued treatment with the anti-CD33 antibody should be administered at the same dose and/or frequency previously administered to the individual.

In other embodiments, the methods of the disclosure comprise providing information indicating that treatment with the anti-CD33 antibody should continue (e.g., at a decreased dose, or at an increased dose; and/or less frequently, or more frequently), e.g., according to any of the methods described herein, for example in Sections A-B above. In other embodiments, the methods of the disclosure comprise providing information indicating that treatment with the anti-CD33 antibody should be adjusted or modified (e.g., by increasing the dose or dosing frequency of the anti-CD33 antibody), e.g., according to any of the methods described herein, for example in Sections A-B above.

In some embodiments, such information is based, at least in part, on the presence or absence of an increase in the levels of sCD33 in a sample obtained from the individual after administration of a dose of an anti-CD33 antibody, as compared to the level of sCD33 in a sample from the individual obtained prior to administration of the anti-CD33 antibody. In some embodiments, such information is based, at least in part, on the presence or absence of an increase in the levels of sCD33 in a sample obtained from the individual after administration of a dose of an anti-CD33 antibody, as compared to the level of sCD33 in a sample from a corresponding individual no treated with the anti-CD33 antibody. In some embodiments, such information may comprise a recommendation to continue treatment of the individual with the anti-CD33 antibody at a decreased dose or an increased dose, and/or less frequently or more frequently, based, at least in part, on determining that treatment with the anti-CD33 antibody should continue (e.g., at a decreased dose, or at an increased dose; and/or less frequently, or more frequently). In some embodiments, such information may comprise a recommendation to continue treatment of the individual with the anti-CD33 antibody at an increased dose, and/or more frequently, based, at least in part, on determining that treatment with the anti-CD33 antibody should be adjusted or modified (e.g., by increasing the dose or dosing frequency of the anti-CD33 antibody). In some embodiments, said recommendation indicates that the dose of anti-CD33 antibody administered to the individual should be increased by any of at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 225-fold, at least about 250-fold, at least about 275-fold, at least about 300-fold, at least about 325-fold, at least about 350-fold, at least about 375-fold, at least about 400-fold, at least about 425-fold, at least about 450-fold, at least about 475-fold, at least about 500-fold, at least about 525-fold, at least about 550-fold, at least about 575-fold, at least about 600-fold, at least about 625-fold, at least about 650-fold, at least about 675-fold, at least about 700-fold, at least about 725-fold, at least about 750-fold, at least about 775-fold, at least about 800-fold, at least about 825-fold, at least about 850-fold, at least about 875-fold, at least about 900-fold, at least about 925-fold, at least about 950-fold, at least about 975-fold, at least about 1000-fold, at least about 1025-fold, at least about 1050-fold, at least about 1075-fold, at least about 1100-fold, at least about 1125-fold, at least about 1150-fold, at least about 1175-fold, at least about 1200-fold, or more, as compared to a prior dose of anti-CD33 antibody administered to the individual. In some embodiments, said recommendation indicates that the frequency of administration of the anti-CD33 antibody to the individual should be increased, e.g., to any of once every eight weeks or more frequently (e.g., about once every 56 days or more frequently), once every seven weeks or more frequently (e.g., about once every 49 days or more frequently), once every six weeks or more frequently (e.g., about once every 42 days or more frequently), once every five weeks or more frequently (e.g., about once every 35 days or more frequently), once every four weeks or more frequently (e.g., about once every 28 days or more frequently), once every three weeks or more frequently (e.g., about once every 21 days or more frequently), once every two weeks or more frequently (e.g., about once every 14 days or more frequently), or once every week (e.g., about once every 7 days or more frequently). In some embodiments, said recommendation indicates that the dose of anti-CD33 antibody administered to the individual should be decreased by any of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, as compared to a prior dose of anti-CD33 antibody administered to the individual. In some embodiments, said recommendation indicates that the frequency of administration of the anti-CD33 antibody to the individual should be decreased, e.g., to any of once every eight weeks or less frequently (e.g., about once every 56 days or less frequently), once every seven weeks or less frequently (e.g., about once every 49 days or less frequently), once every six weeks or less frequently (e.g., about once every 42 days or less frequently), once every five weeks or less frequently (e.g., about once every 35 days or less frequently), once every four weeks or less frequently (e.g., about once every 28 days or more frequently), once every three weeks or less frequently (e.g., about once every 21 days or less frequently), once every two weeks or less frequently (e.g., about once every 14 days or less frequently), or once every week (e.g., about once every 7 days or less frequently). In some embodiments, said recommendation indicates that any of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, or more, additional doses of the anti-CD33 antibody should be administered to the individual, e.g., at an increased dose, at a decreased dose, at an increased frequency, and/or at a decreased frequency, e.g., as described above. In some embodiments, said recommendation indicates that the individual should continue to receive treatment with the anti-CD33 antibody for at least about 1 week, at least about 2 weeks, at least about 3 weeks, at least about 4 weeks, at least about 1 month, at least 2 months, at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, at least 24 months, at least 25 months, at least 26 months, at least 27 months, at least 28 months, at least 29 months, at least 30 months, at least 31 months, at least 32 months, at least 33 months, at least 34 months, at least 35 months, at least 36 months, at least 37 months, at least 38 months, at least 39 months, at least 40 months, at least 41 months, at least 42 months, at least 43 months, at least 44 months, at least 45 months, at least 46 months, at least 47 months, at least 48 months, at least 49 months, at least 50 months, at least 51 months, at least 52 months, at least 53 months, at least 54 months, at least 55 months, at least 56 months, at least 57 months, at least 58 months, at least 59 months, at least 60 months, at least 61 months, at least 62 months, at least 63 months, at least 64 months, at least 65 months, at least 66 months, at least 67 months, at least 68 months, at least 69 months, at least 70 months, at least 71 months, at least 72 months, at least 73 months, at least 74 months, at least 75 months, at least 76 months, at least 77 months, at least 78 months, at least 79 months, at least 80 months, at least 81 months, at least 82 months, at least 83 months, at least 84 months, at least 85 months, at least 86 months, at least 87 months, at least 88 months, at least 89 months, at least 90 months, at least 91 months, at least 92 months, at least 93 months, at least 94 months, at least 95 months, at least 96 months, at least 97 months, at least 98 months, at least 99 months, at least 100 months, at least 101 months, at least 102 months, at least 103 months, at least 104 months, at least 105 months, at least 106 months, at least 107 months, at least 108 months, at least 109 months, at least 110 months, at least 111 months, at least 112 months, at least 113 months, at least 114 months, at least 115 months, at least 116 months, at least 117 months, at least 118 months, at least 119 months, at least 120 months, or more, e.g., at an increased dose, at a decreased dose, at an increased frequency, and/or at a decreased frequency, e.g., as described above.

In some embodiments, said information is provided to the individual (e.g., the individual being treated with the anti-CD33 antibody), a caregiver (for example, without limitation, a family caregiver, a professional caregiver, a private caregiver, an independent caregiver, a volunteer caregiver, an assisted living community, a nursing home or facility, a hospice facility, a nurse, a physician's assistant, a nurse's assistant, a guardian, a conservator, a surrogate, and the like), a physician (for example, without limitation, a primary care doctor, a geriatrician, a geriatric psychiatrist, a geropsychologist, a neurologist, a neuropsychologist, a specialist [e.g., specializing in a disease or injury described herein], and the like), a hospital, a clinic (for example, without limitation, a primary care clinic, a specialized clinic [e.g., specializing in a disease or injury described herein], a memory clinic or center, a mental health clinic, a community health center or clinic, a retail clinic, a rural clinic, a mobile clinic, and the like), a third-party payer (for example, without limitation, a government agency, an insurance company or organization, and the like), an insurance company or organization (for example, without limitation, an indemnity policy, a preferred provider organization [PPO], a health maintenance organization [HMO], a self-insured health plan, a single employer self-insured plan, an exclusive provider organization [EPO], a high deductible health plan [HDHP], an insurance service or provider under Consolidated Omnibus Budget Reconciliation Act (COBRA), an insurance company or organization in a country other than the United States, and the like), or a government office or health program (for example, without limitation, Medicare, Medicaid, State Children's Health Insurance Program (SCHIP), Department of Defense [DOD] TRICARE, Veterans Health Administration [VHA], Indian Health Service [HIS], a state health program or agency, a health program or agency in a country other than the United States, and the like). In some embodiments, said information is provided as a report. In some embodiments, the methods provided herein comprise generating a report comprising said information. In some embodiments, said information and/or said report is provided in an electronic, web-based, or paper form.

### D. Diseases or Injuries

As disclosed herein, increases in sCD33 levels in an individual (e.g., in blood or cerebrospinal fluid) after administration of an anti-CD33 antibody may be used for assessing the activity or therapeutic efficacy of the anti-CD33 antibody in an individual having a disease or injury, and for monitoring, guiding, or adjusting treatment with an anti-CD33 antibody in an individual having a disease or injury. In some embodiments, the disease or injury is dementia, frontotemporal dementia, Alzheimer's disease, Parkinson's disease, amyotrophic lateral sclerosis, multiple sclerosis, Huntington's disease, vascular dementia, mixed dementia, and taupathy disease.

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include behavioral symptoms, cognitive symptoms, confusion, irritability and aggression, mood swings, trouble with language, and long-term memory loss. As the disease progresses, bodily functions are lost, ultimately leading to death. Alzheimer's disease develops for an unknown and variable amount of time before becoming fully apparent, and it can progress undiagnosed for years.

A growing body of evidence indicates that CD33 is involved in AD. For example, large-scale genome-wide association studies (GWAS) have identified CD33 as a susceptibility locus for late-onset AD (LOAD). One allele of CD33, the rs3865444C risk allele, is associated with (1) increased cell surface expression of CD33 on monocytes; (2) reduced uptake of Aβ42; (3) greater neuritic amyloid plaque burden and fibrillary amyloid on in vivo imaging; and (4) increased numbers of activated human microglia in vivo (see, e.g., Bradshaw E M, et al. Nat Neurosci. 2013; 16 (7): 848-850). In contrast, the CD33 rs3865444A allele variant is associated with reduced CD33 expression and confers protection against LOAD (see, e.g., Naj A C, et al. Nat Genet. 2011; 43 (5): 436-441; Hollingworth P, et al. Nat Genet. 2011; 43 (5): 429-435; Jiang Y T, et al. Ann Transl Med. 2018; 6 (10): 169). Another single nucleotide polymorphism (SNP), rs12459419, which is in strong linkage disequilibrium (i.e., coinherited) with rs3865444, appears to be the causal SNP modulating the alternative splicing of CD33 exon 2 and thus influencing susceptibility to AD (see, e.g., Malik M, et al. J Neurosci. 2013; 33 (33): 13320-13325; Raj T, et al. Hum Mol Genet. 2014; 23 (10): 2729-2736). Finally, transgenic expression of human full-length CD33M in murine microglia inhibits phagocytosis (see, e.g., Bhattacherjee A, et al. Commun Biol. 2019; 2 (450).

In some embodiments, the disease or injury according to any of the methods of the disclosure is Alzheimer's disease. In some embodiments, the Alzheimer's disease is early onset Alzheimer's disease, late onset Alzheimer's disease, or familial Alzheimer's disease. In some embodiments, the Alzheimer's disease is pre-clinical, early stage, middle stage, or late stage Alzheimer's disease. In some embodiments, the Alzheimer's disease is mild, moderate, mild to moderate, or severe Alzheimer's disease. In some embodiments, the Alzheimer's disease is mild to moderate Alzheimer's disease, e.g., including one or more, or all, of a clinical diagnosis of probable Alzheimer's disease dementia based on National Institute on Aging Alzheimer's Association criteria, a Mini-Mental State Examination (MMSE) prior to treatment with an anti-CD33 antibody of between 16 and 28 points, a Clinical Dementia Rating-Global Score (CDR-GS) prior to treatment with an anti-CD33 antibody of 0.5, 1.0, or 2.0, and/or a positive amyloid-PET scan by qualitative read. See, e.g., McKhann et al., Alzheimers Dement (2011) 7 (3): 263-269 for additional information on diagnostic guidelines for Alzheimer's disease.

In some embodiments, an individual according to any of the methods of the disclosure is a human having Alzheimer's disease, e.g., early onset Alzheimer's disease, late onset Alzheimer's disease, or familial Alzheimer's disease. In some embodiments, the individual has pre-clinical early stage, middle stage, or late stage Alzheimer's disease. In some embodiments, the individual has mild, moderate, mild to moderate, or severe Alzheimer's disease.

In some embodiments, the individual has mild to moderate Alzheimer's disease, e.g., including one or more, or all, of a clinical diagnosis of probable Alzheimer's disease dementia based on National Institute on Aging Alzheimer's Association criteria, a Mini-Mental State Examination (MMSE) prior to treatment with an anti-CD33 antibody of between 16 and 28 points, a Clinical Dementia Rating-Global Score (CDR-GS) prior to treatment with an anti-CD33 antibody of 0.5, 1.0, or 2.0, and/or a positive amyloid-PET scan by qualitative read. In some embodiments, the individual is an adult, e.g., with an age of at least about 50 years, or an age of between about 50 and about 85 years. In some embodiments, the individual is taking a stable dose of cholinesterase inhibitor and/or memantine therapy for Alzheimer's disease prior to treatment with the anti-CD33 antibody (e.g., for at least 4 weeks prior to the start of treatment with the anti-CD33 antibody, and optionally, with no intent to initiate, discontinue, or alter the dose of any therapy for Alzheimer's disease for the duration of treatment with the anti-CD33 antibody). In some embodiments, the individual does not have two copies of the minor allele rs12459419T. In some embodiments, the individual does not have, or does not have a history of, central nervous system or systemic autoimmune disorders including, but not limited to, rheumatoid arthritis, multiple sclerosis, lupus erythematosus, anti-phospholipid antibody syndrome, or Behçet disease. In some embodiments, the individual does not have dementia due to a condition other than Alzheimer's disease, including, but not limited to, Frontotemporal Dementia, Parkinson's disease, dementia with Lewy bodies, Huntington disease, or vascular dementia. In some embodiments, the individual does not have, or does not have a history of, clinically evident vascular disease potentially affecting the brain (e.g., clinically significant carotid, vertebral stenosis or plaque, aortic aneurysm, intracranial aneurysm, cerebral hemorrhage, arteriovenous malformation) that has the potential to affect cognitive function. In some embodiments, the individual does not have, or does not have a history of, stroke within the past 2 years prior to the start of treatment with the anti-CD33 antibody. In some embodiments, the individual does not have, or does not have a history of, transient ischemic attack within the last 12 months prior to the start of treatment with the anti-CD33 antibody. In some embodiments, the individual does not have, or does not have a history of, severe, clinically significant (e.g., persistent neurologic deficit or structural brain damage) central nervous system trauma (e.g., cerebral contusion). In some embodiments, the individual does not have MRI evidence of more than two lacunar infarcts, any territorial infarct >1 cm³, or significant hyperintense on fluid attenuated inversion recovery (FLAIR) hyperintense lesions in the cerebral white matter that may contribute to cognitive dysfunction.

### Dementia

Dementia is a non-specific syndrome (i.e., a set of signs and symptoms) that presents as a serious loss of global cognitive ability in a previously unimpaired person, beyond what might be expected from normal ageing. Dementia may be static, as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia. Exemplary forms of dementia include, without limitation, frontotemporal dementia, Alzheimer's disease, vascular dementia, mixed dementia, semantic dementia, and dementia with Lewy bodies.

### Frontotemporal Dementia

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene.

### Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

### Amyotrophic Lateral Sclerosis (ALS)

As used herein, amyotrophic lateral sclerosis (ALS), or motor neuron disease, or Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

It has been shown that Progranulin plays a role in ALS (Schymick, J C et al., (2007) J Neurol Neurosurg Psychiatry.; 78:754-6) and protects again the damage caused by ALS causing proteins such as TDP-43 (Laird, A S et al., (2010). PLOS ONE 5: e13368). It was also demonstrated that pro-NGF induces p75 mediated death of oligodendrocytes and corticospinal neurons following spinal cord injury (Beatty et al., Neuron (2002), 36, pp. 375-386; Giehl et al, Proc. Natl. Acad. Sci USA (2004), 101, pp 6226-30).

### Huntington's Disease

Huntington's disease (HD) is an inherited neurodegenerative disease caused by an autosomal dominant mutation in the Huntingtin gene (HTT). Expansion of a cytokine-adenine-guanine (CAG) triplet repeat within the Huntingtin gene results in production of a mutant form of the Huntingtin protein (Htt) encoded by the gene. This mutant Huntingtin protein (mHtt) is toxic and contributes to neuronal death. Symptoms of Huntington's disease most commonly appear between the ages of 35 and 44, although they can appear at any age.

Symptoms of Huntington's disease, include, without limitation, motor control problems, jerky, random movements (chorea), abnormal eye movements, impaired balance, seizures, difficulty chewing, difficulty swallowing, cognitive problems, altered speech, memory deficits, thinking difficulties, insomnia, fatigue, dementia, changes in personality, depression, anxiety, and compulsive behavior.

### Taupathy Disease

Taupathy diseases, or tauopathies, are a class of neurodegenerative disease caused by aggregation of the microtubule-associated protein tau within the brain. Alzheimer's disease (AD) is the most well-known taupathy disease, and involves an accumulation of tau protein within neurons in the form of insoluble neurofibrillary tangles (NFTs). Other taupathy diseases and disorders include progressive supranuclear palsy, dementia pugilistica (chromic traumatic encephalopathy), frontotemporal dementia, parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, and frontotemporal lobar degeneration.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. MS affects the ability of nerve cells in the brain and spinal cord to communicate with each other effectively. Nerve cells communicate by sending electrical signals called action potentials down long fibers called axons, which are contained within an insulating substance called myelin. In MS, the body's own immune system attacks and damages the myelin. When myelin is lost, the axons can no longer effectively conduct signals. MS onset usually occurs in young adults, and is more common in women.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoff's phenomenon, which is an exacerbation of extant symptoms due to an exposure to higher than usual ambient temperatures; and Lhermitte's sign, which is an electrical sensation that runs down the back when bending the neck.

In some embodiments of any of the methods of the disclosure, a subject or individual is a mammal. Mammals include, without limitation, domesticated animals (e.g., cows, sheep, cats, dogs, and horses), primates (e.g., humans and non-human primates such as monkeys), rabbits, and rodents (e.g., mice and rats). In some embodiments, the subject or individual is a human.

### E. Exemplary Anti-CD33 Antibodies

### (i) Anti-CD33 Antibody Activities

### Reducing CD33 Levels

In some embodiments, anti-CD33 antibodies that may be used in the methods of the disclosure exhibit the ability to decrease cellular levels of CD33. Cellular levels of CD33 may refer to, without limitation, cell surface levels of CD33, intracellular levels of CD33, and total levels of CD33. In some embodiments, a decrease in cellular levels of CD33 comprises decrease in cell surface levels of CD33. In some embodiments, anti-CD33 antibodies that may be used in the methods of the disclosure decrease cellular levels of CD33 (e.g., cell surface levels of CD33) and have one or more of the following characteristics: (1) inhibits or reduces one or more CD33 activities; (2) the ability to inhibit or reduce binding of a CD33 to one or more of its ligands; (3) the ability to reduce CD33 expression in CD33-expressing cells; (4) the ability to interact, bind, or recognize a CD33 protein; (5) the ability to specifically interact with or bind to a CD33 protein; and (6) the ability to treat, ameliorate, or prevent any aspect of a disease or disorder described or contemplated herein.

In some embodiments, anti-CD33 antibodies that may be used in the methods of the present disclosure decrease cellular levels of CD33 (e.g., cell surface levels, intracellular levels, and/or total levels). In some embodiments, anti-CD33 antibodies that may be used in the methods of the present disclosure induce downregulation of CD33. In some embodiments, anti-CD33 antibodies that may be used in the methods of the present disclosure induce cleavage of CD33. In some embodiments, anti-CD33 antibodies that may be used in the methods of the present disclosure induce internalization of CD33. In some embodiments, anti-CD33 antibodies that may be used in the methods of the present disclosure induce shedding of CD33. In some embodiments, anti-CD33 antibodies that may be used in the methods of the present disclosure induce degradation of CD33. In some embodiments, anti-CD33 antibodies that may be used in the methods of the present disclosure induce desensitization of CD33. In some embodiments, anti-CD33 antibodies that may be used in the methods of the present disclosure act as a ligand mimetic to transiently activate CD33. In some embodiments, anti-CD33 antibodies that may be used in the methods of the present disclosure act as a ligand mimetic and transiently activate CD33 before inducing a decrease in cellular levels of CD33 and/or inhibition of interaction (e.g., binding) between CD33 and one or more CD33 ligands. In some embodiments, anti-CD33 antibodies that may be used in the methods of the present disclosure act as a ligand mimetic and transiently activate CD33 before inducing degradation of CD33. In some embodiments, anti-CD33 antibodies that may be used in the methods of the present disclosure act as a ligand mimetic and transiently activate CD33 before inducing cleavage of CD33. In some embodiments, anti-CD33 antibodies that may be used in the methods of the present disclosure act as a ligand mimetic and transiently activate CD33 before inducing internalization of CD33. In some embodiments, anti-CD33 antibodies that may be used in the methods of the present disclosure act as a ligand mimetic and transiently activates CD33 before inducing shedding of CD33. In some embodiments, anti-CD33 antibodies that may be used in the methods of the present disclosure act as a ligand mimetic and transiently activate CD33 before inducing downregulation of CD33 expression. In some embodiments, anti-CD33 antibodies that may be used in the methods of the present disclosure act as a ligand mimetic and transiently activate CD33 before inducing desensitization of CD33.

In some embodiments, anti-CD33 antibodies that may be used in the methods of the present disclosure decrease cellular levels of CD33 (e.g., cell surface levels, intracellular levels, and/or total levels) in vitro, e.g., cellular levels of CD33 (e.g., cell surface levels, intracellular levels, and/or total levels) in one or more cells that express CD33, such as monocytes, macrophages, T cells, dendritic cells, microglia, bone marrow-derived dendritic cells, M1 microglia, activated M1 microglia, M2 microglia, M1 macrophages, activated M1 macrophages, and/or M2 macrophages, neutrophils, osteoclasts, T helper cells, cytotoxic T cells, and/or granulocytes. In some embodiments, anti-CD33 antibodies that may be used in the methods of the disclosure exhibit the ability to decrease cellular levels of CD33 (e.g., cell surface levels, intracellular levels, and/or total levels) in vitro by any of at least about 10%, at least about 20%, at least about 30%, at least about 40%, at least about 50%, at least about 60%, at least about 70%, at least about 80%, at least about 90%, or 100%, for example, as compared to cellular levels of CD33 without the anti-CD33 antibody, or as compared to a control antibody such as an isotype control antibody.

In some embodiments, anti-CD33 antibodies that may be used in the methods of the disclosure exhibit the ability to decrease cellular levels of CD33 (e.g., cell surface levels, intracellular levels, and/or total levels) in vivo, e.g., in one or more cells that express CD33, such as monocytes, macrophages, T cells, dendritic cells, microglia, bone marrow-derived dendritic cells, M1 microglia, activated M1 microglia, M2 microglia, M1 macrophages, activated M1 macrophages, and/or M2 macrophages, neutrophils, osteoclasts, T helper cells, cytotoxic T cells, and/or granulocytes, in an individual administered the anti-CD33 antibody, e.g., according to any treatment regimen described herein. In some embodiments, anti-CD33 antibodies that may be used in the methods of the disclosure exhibit the ability to decrease cellular levels of CD33 (e.g., cell surface levels, intracellular levels, and/or total levels) in vivo by any of at least about 10%, at least about 20%, at least about 30%, at least about 40%, at least about 50%, at least about 60%, at least about 70%, at least about 80%, at least about 90%, or 100%, for example, as compared to cellular levels of CD33 without administration of the anti-CD33 antibody (e.g., prior to administration of the anti-CD33 antibody), or as compared to a control antibody such as an isotype control antibody. In some embodiments, cellular levels of CD33 (e.g., cell surface levels, intracellular levels, and/or total levels) in vivo, e.g., in one or more cells that express CD33, such as monocytes, macrophages, T cells, dendritic cells, microglia, bone marrow-derived dendritic cells, M1 microglia, activated M1 microglia, M2 microglia, M1 macrophages, activated M1 macrophages, and/or M2 macrophages, neutrophils, osteoclasts, T helper cells, cytotoxic T cells, and/or granulocytes, in an individual are assessed in one or more samples obtained from the individual, such as a cerebrospinal fluid sample or a blood sample (e.g., a whole blood, plasma or serum sample).

Anti-CD33 antibodies that may be used in the methods of the present disclosure may decrease cellular levels (e.g., cell surface levels) of CD33 with a half-maximal effective concentration (EC50) (e.g., when measured in vitro using primary human dendritic cells) in the picomolar range. In certain embodiments, the EC50 of the antibody is about 0.1 to about 500 pM. In certain embodiments, the EC50 of the antibody is about 1 to about 250 pM. In certain embodiments, the EC50 of the antibody is about 4.1 to about 151.1 pM. In some embodiments, the EC50 of the antibody is less than about or equal to about 500 pM, 400 pM, 300 pM, 250 pM, 225 pM, 200 pM, 175 pM, 150 pM, 125 pM, 100 pM, 75 pM, 50 pM, 25 pM, 10 pM, 1 pM, or 0.5 pM. In some embodiments, the EC50 of the antibody is less than about 74.3 pM. In some embodiments, the EC50 of the antibody is greater than about or equal to about 0.1 pM, 0.5 pM, 1 pM, 10 pM, 25 pM, 50 pM, 75 pM, 100 pM, 125 pM, 150 pM, 175 pM, 200 pM, 225 pM, 250 pM, 300 pM, or 400 pM. That is, the EC50 of the antibody can be any of a range having an upper limit of about 500 pM, 400 pM, 300 pM, 250 pM, 225 pM, 200 pM, 175 pM, 150 pM, 125 pM, 100 pM, 75 pM, 50 pM, 25 pM, 10 pM, 1 pM, or 0.5 pM, and an independently selected lower limit of about 0.1 pM, 0.5 pM, 1 pM, 10 pM, 25 pM, 50 pM, 75 pM, 100 pM, 125 pM, 150 pM, 175 pM, 200 pM, 225 pM, 250 pM, 300 pM, or 400 pM, wherein the lower limit is less than the upper limit. In some embodiments, the EC50 of the antibody is any of about 1 pM, 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 5-pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 160 pM, 165 pM, 170 pM, 175 pM, 180 pM, 185 pM, 190 pM, 195 pM, or 200 pM. Various methods of measuring antibody EC50 values are known in the art, including, for example, by flow cytometry. In some embodiments, the EC50 is measured in vitro using primary human dendritic cells. In some embodiments, the EC50 is measured in vitro using primary human monocytes. In some embodiments, the EC50 is measured in vitro using primary human macrophages. In some embodiments, the EC50 is measured in vitro using cultured cells transfected with human CD33. In some embodiments, the EC50 is measured at a temperature of approximately 4° C. In some embodiments, the EC50 is measured at a temperature of approximately 25° C. In some embodiments, the EC₅₀ is measured at a temperature of approximately 35° C. In some embodiments, the EC50 is measured at a temperature of approximately 37° C. In some embodiments, the EC50 is determined using a monovalent antibody (e.g., a Fab) or a full-length antibody in a monovalent form. In some embodiments, the EC50 is determined using antibodies containing constant regions that demonstrate enhanced Fc receptor binding. In some embodiments, the EC50 is determined using antibodies containing constant regions that demonstrate reduced Fc receptor binding.

In some embodiments, anti-CD33 antibodies that may be used in the methods of the disclosure inhibit cell surface clustering of CD33.

In some embodiments, decreasing cellular levels of CD33 by an anti-CD33 antibody, e.g., anti-CD33 antibodies that may be used in the methods of the disclosure, results in the reduction, neutralization, prevention, or curbing of one or more CD33 activities, including, without limitation, reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; reducing proliferation of monocytes, macrophages, T cells, dendritic cells, neutrophils, and/or microglia; reducing the overall functionality of monocytes, macrophages, T cells, dendritic cells, neutrophils, and/or microglia; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, and multiple sclerosis; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; modulated expression of one or more inflammatory receptors, such as CD86, expressed on one or more of microglia, macrophages, dendritic cells, bone marrow-derived dendritic cells, neutrophils, T cells, T helper cells, or cytotoxic T cells; enhancing infiltration of one or more of immunosuppressor dendritic cells, immunosuppressor macrophages, myeloid derived suppressor cells, tumor-associated macrophages, immunosuppressor neutrophils, and regulatory T cells into tumors; increasing number of tumor-promoting myeloid/granulocytic immune-suppressive cells in a tumor, in peripheral blood, or other lymphoid organ; enhancing tumor-promoting activity of myeloid-derived suppressor cells; decreasing activation of tumor-specific T lymphocytes with tumor killing potential; decreasing infiltration of tumor-specific T lymphocytes with tumor killing potential; increasing tumor growth rate; increasing rate of tumor recurrence; decreasing efficacy of one or more immune-therapies that modulate anti-tumor T cell responses, optionally wherein the one or more immune-therapies are immune-therapies that target one or more proteins selected from CD40, OX40, ICOS, CD28, CD137/4-1BB, CD27, GITR, PD-L1, CTLA4, PD-L2, PD-1, B7-H3, B7-H4, HVEM, LIGHT, BTLA, VISTA, KIR, GAL9, TIM1, TIM3, TIM4, A2AR, LAG3, DR-5, CD39, CD70, TREM1, TREM2, Siglec-5, Siglec-7, Siglec-9, Siglec-11, SirpA, CD447, CSF-1 receptor, and any combination thereof, or of one or chemotherapy agents and/or more cancer vaccines.

Any suitable methods known in the art may be used to determine cellular levels of CD33, e.g., in one or more cells that express CD33, such as monocytes, macrophages, T cells, dendritic cells, microglia, bone marrow-derived dendritic cells, M1 microglia, activated M1 microglia, M2 microglia, M1 macrophages, activated M1 macrophages, and/or M2 macrophages, neutrophils, osteoclasts, T helper cells, cytotoxic T cells, and/or granulocytes, such as immunoblots (e.g., Western blots), mass spectrometry, flow cytometry, an immunoassay (e.g., a SIMOA assay from Quanterix, see, e.g., the website: www.quanterix.com/simoa-technology/), a proximity extension assay (e.g., an assay from Olink, see, e.g., the website: www.olink.com/our-platform/our-pea-technology), an electrochemiluminescence-based assay (e.g., an assay from Meso Scale Diagnostics, see, e.g., the website: www.mesoscale.com/en/technical_resources/our_technology/ecl), and/or aptamer-based methods such as SOMASCAN assay (see. e.g., Candia et al. (2017) Sci Rep 7, 14248). In some embodiments, cell surface level of CD33 may be determined according to any method known in the art. In some embodiments, the cell surface level of CD33 may be determined using flow cytometry. In some embodiments, the cell surface level of CD33 is expressed as Mean Fluorescence Intensity (MFI). In some embodiments, the cell surface level of CD33 is expressed as Molecules of Equivalent Soluble Fluorochrome (MESF) (e.g., see Schawrts et al., (2004) Clin Cytometry 57B: 1-6).

### Increasing Soluble CD33 Levels

In some embodiments, anti-CD33 antibodies that may be used in the methods of the disclosure exhibit the ability to increase levels of soluble CD33 (sCD33).

In some embodiments, anti-CD33 antibodies that may be used in the methods of the disclosure exhibit the ability to increase levels of sCD33 in vitro, e.g., sCD33 levels produced from one or more cells that express CD33, such as monocytes, macrophages, T cells, dendritic cells, microglia, bone marrow-derived dendritic cells, M1 microglia, activated M1 microglia, M2 microglia, M1 macrophages, activated M1 macrophages, and/or M2 macrophages, neutrophils, osteoclasts, T helper cells, cytotoxic T cells, and/or granulocytes. In some embodiments, anti-CD33 antibodies that may be used in the methods of the disclosure exhibit the ability to increase levels of sCD33 in vitro by any of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, at least about 600%, at least about 610%, at least about 620%, at least about 630%, at least about 640%, at least about 650%, at least about 660%, at least about 670%, at least about 680%, at least about 690%, at least about 700%, at least about 710%, at least about 720%, at least about 730%, at least about 740%, at least about 750%, at least about 760%, at least about 770%, at least about 780%, at least about 790%, at least about 800%, at least about 810%, at least about 820%, at least about 830%, at least about 840%, at least about 850%, at least about 860%, at least about 870%, at least about 880%, at least about 890%, at least about 900%, at least about 910%, at least about 920%, at least about 930%, at least about 940%, at least about 950%, at least about 960%, at least about 970%, at least about 980%, at least about 990%, at least about 1000%, or more, for example, as compared to sCD33 levels without the anti-CD33 antibody, or as compared to a control antibody such as an isotype control antibody. In some embodiments, anti-CD33 antibodies that may be used in the methods of the disclosure exhibit the ability to increase levels of sCD33 in vitro by any of at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 225-fold, at least about 250-fold, at least about 275-fold, at least about 300-fold, at least about 325-fold, at least about 350-fold, at least about 375-fold, at least about 400-fold, at least about 425-fold, at least about 450-fold, at least about 475-fold, at least about 500-fold, at least about 525-fold, at least about 550-fold, at least about 575-fold, at least about 600-fold, at least about 625-fold, at least about 650-fold, at least about 675-fold, at least about 700-fold, at least about 725-fold, at least about 750-fold, at least about 775-fold, at least about 800-fold, at least about 825-fold, at least about 850-fold, at least about 875-fold, at least about 900-fold, at least about 925-fold, at least about 950-fold, at least about 975-fold, at least about 1000-fold, at least about 1025-fold, at least about 1050-fold, at least about 1075-fold, at least about 1100-fold, at least about 1125-fold, at least about 1150-fold, at least about 1175-fold, at least about 1200-fold, or more, for example, as compared to sCD33 levels without the anti-CD33 antibody, or as compared to a control antibody such as an isotype control antibody.

In some embodiments, anti-CD33 antibodies that may be used in the methods of the disclosure exhibit the ability to increase levels of sCD33 in vivo, e.g., in an individual administered the anti-CD33 antibody, e.g., according to any treatment regimen described herein. In some embodiments, anti-CD33 antibodies that may be used in the methods of the disclosure exhibit the ability to increase levels of sCD33 in vivo by any of at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200%, at least about 210%, at least about 220%, at least about 230%, at least about 240%, at least about 250%, at least about 260%, at least about 270%, at least about 280%, at least about 290%, at least about 300%, at least about 310%, at least about 320%, at least about 330%, at least about 340%, at least about 350%, at least about 360%, at least about 370%, at least about 380%, at least about 390%, at least about 400%, at least about 410%, at least about 420%, at least about 430%, at least about 440%, at least about 450%, at least about 460%, at least about 470%, at least about 480%, at least about 490%, at least about 500%, at least about 510%, at least about 520%, at least about 530%, at least about 540%, at least about 550%, at least about 560%, at least about 570%, at least about 580%, at least about 590%, at least about 600%, at least about 610%, at least about 620%, at least about 630%, at least about 640%, at least about 650%, at least about 660%, at least about 670%, at least about 680%, at least about 690%, at least about 700%, at least about 710%, at least about 720%, at least about 730%, at least about 740%, at least about 750%, at least about 760%, at least about 770%, at least about 780%, at least about 790%, at least about 800%, at least about 810%, at least about 820%, at least about 830%, at least about 840%, at least about 850%, at least about 860%, at least about 870%, at least about 880%, at least about 890%, at least about 900%, at least about 910%, at least about 920%, at least about 930%, at least about 940%, at least about 950%, at least about 960%, at least about 970%, at least about 980%, at least about 990%, at least about 1000%, or more, for example, as compared to sCD33 levels without administration of the anti-CD33 antibody (e.g., prior to administration of the anti-CD33 antibody), or as compared to a control antibody such as an isotype control antibody. In some embodiments, anti-CD33 antibodies that may be used in the methods of the disclosure exhibit the ability to increase levels of sCD33 in vivo by any of at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 125-fold, at least about 150-fold, at least about 175-fold, at least about 200-fold, at least about 225-fold, at least about 250-fold, at least about 275-fold, at least about 300-fold, at least about 325-fold, at least about 350-fold, at least about 375-fold, at least about 400-fold, at least about 425-fold, at least about 450-fold, at least about 475-fold, at least about 500-fold, at least about 525-fold, at least about 550-fold, at least about 575-fold, at least about 600-fold, at least about 625-fold, at least about 650-fold, at least about 675-fold, at least about 700-fold, at least about 725-fold, at least about 750-fold, at least about 775-fold, at least about 800-fold, at least about 825-fold, at least about 850-fold, at least about 875-fold, at least about 900-fold, at least about 925-fold, at least about 950-fold, at least about 975-fold, at least about 1000-fold, at least about 1025-fold, at least about 1050-fold, at least about 1075-fold, at least about 1100-fold, at least about 1125-fold, at least about 1150-fold, at least about 1175-fold, at least about 1200-fold, or more, for example, as compared to sCD33 levels without administration of the anti-CD33 antibody (e.g., prior to administration of the anti-CD33 antibody), or as compared to a control antibody such as an isotype control antibody. In some embodiments, levels of sCD33 in an individual are assessed in one or more samples obtained from the individual, such as a cerebrospinal fluid sample or a blood sample (e.g., a whole blood, plasma or serum sample).

Any suitable methods known in the art may be used to determine the levels of sCD33 in a sample. For example, sCD33 levels in a sample (e.g., a cerebrospinal fluid sample, or a blood sample such as a whole blood, serum or plasma sample) may be determined using immunoblots (e.g., Western blots), mass spectrometry, flow cytometry, an immunoassay (e.g., a SIMOA assay from Quanterix, see, e.g., the website: www.quanterix.com/simoa-technology/), a proximity extension assay (e.g., an assay from Olink, see, e.g., the website: www.olink.com/our-platform/our-pea-technology), an electrochemiluminescence-based assay (e.g., an assay from Meso Scale Diagnostics, see, e.g., the website: www.mesoscale.com/en/technical_resources/our_technology/ecl), and/or aptamer-based methods such as SOMASCAN assay (see. e.g., Candia et al. (2017) Sci Rep 7, 14248).

In some embodiments, increases in sCD33 levels in vitro (e.g., sCD33 levels produced from one or more cells that express CD33, such as one or more microglia) or in vivo (e.g., in an individual administered an anti-CD33 antibody) may be indicative of microglial activation.

Inhibiting Interaction of CD33 with One or More CD33 Ligands

In some embodiments, anti-CD33 antibodies that may be used in the methods of the disclosure inhibit interaction (e.g., binding) between a CD33 protein of the present disclosure and one or more CD33 ligands including, without limitation, CD33 ligands expressed on red blood cells, CD33 ligands expressed on bacterial cells, CD33 ligands expressed on apoptotic cells, CD33 ligands expressed on tumor cells, CD33 ligands expressed on viruses, CD33 ligands expressed on dendritic cells, CD33 ligands expressed on nerve cells, CD33 ligands expressed on glial cells, CD33 ligands expressed on microglia, CD33 ligands expressed on astrocytes, CD33 ligands on beta amyloid plaques, CD33 ligands on Tau tangles, CD33 ligands on disease-causing proteins, CD33 ligands on disease-causing peptides, CD33 ligands expressed on macrophages, CD33 ligands expressed on natural killer cells, CD33 ligands expressed on T cells, CD33 ligands expressed on T helper cells, CD33 ligands expressed on cytotoxic T cells, CD33 ligands expressed on B cells, CD33 ligands expressed on tumor-imbedded immunosuppressor dendritic cells, CD33 ligands expressed on tumor-imbedded immunosuppressor macrophages, CD33 ligands expressed on myeloid-derived suppressor cells, CD33 ligands expressed on regulatory T cells, secreted mucins, sialic acid, sialic acid-containing glycolipids, sialic acid-containing glycoproteins, alpha-2,6-linked sialic acid-containing glycolipids, alpha-2,6-linked sialic acid-containing glycoproteins, alpha-2,3-linked sialic acid-containing glycolipids, alpha-2,3-linked sialic acid-containing glycoproteins, alpha-1-acid glycoprotein (AGP), CD24 protein, and gangliosides.

In some embodiments, anti-CD33 antibodies that may be used in the methods of the disclosure bind to a CD33 protein of the present disclosure expressed on the surface of a cell, and the antibodies inhibit interaction (e.g., binding) between the CD33 protein and one or more CD33 ligands. In some embodiments, anti-CD33 antibodies that may be used in the methods of the disclosure bind to a CD33 protein of the present disclosure and inhibit interaction (e.g., binding) between the CD33 protein and one or more CD33 ligands by reducing the effective levels of CD33 that is available to interact with these ligands either on the cell surface or inside the cell. In some embodiments, anti-CD33 antibodies that may be used in the methods of the disclosure bind to a CD33 protein of the present disclosure and inhibit interaction (e.g., binding) between the CD33 protein and one or more CD33 ligands by inducing degradation of CD33.

Any in vitro cell-based assays or suitable in vivo model described herein or known in the art may be used to measure inhibition of interaction (e.g., binding) between CD33 and one or more CD33 ligands. In some embodiments, anti-CD33 antibodies of the present disclosure inhibit interaction (e.g., binding) between CD33 and one or more CD33 ligands by at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more at saturating antibody concentrations utilizing any in vitro assay or cell-based culture assay described herein or known in the art

### Inhibition of CD33 Activities

In some embodiments, anti-CD33 antibodies that may be used in the methods of the disclosure inhibit one or more activities of a CD33 protein, including, without limitation, counteracting one or more of phosphorylation of Tyr-340 and Tyr-358 by a Src family tyrosine kinase, such as LCK and FYN; recruitment of and binding to the tyrosine-specific protein phosphatases SHP1 and SHP2; recruitment of and binding to PLC-gamma1, which acts as a guanine nucleotide exchange factor for Dynamini-1; recruitment of and binding to SH2-domain containing protein (e.g., Crk1); recruitment of and binding to the spleen tyrosine kinase Syk; recruitment of and binding to SH3-SH2-SH3 growth factor receptor-bound protein 2 (Grb2); recruitment of and binding to multiple SH2-containing proteins; phosphorylation of Ser-307 and Ser-342 by protein kinase C; modulated expression of one or more anti-inflammatory cytokines, IL-4, IL-10, IL-13, IL-35, IL-16, TGF-beta, IL-1Ra, G-CSF, and soluble receptors for TNF, IFN-beta1a, IFN-beta1b, or IL-6 in monocytes, macrophages, T cells, dendritic cells neutrophils, and/or microglia; decreasing intracellular calcium mobilization; modulated expression of one or more pro-inflammatory cytokines IFN-α4, IFN-b, IL-1B, TNF-α, IL-6, IL-8, CRP, IL-20 family members, LIF, IFN-gamma, OSM, CNTF, GM-CSF, IL-11, IL-12, IL-17, IL-18, IL-23, CXCL10, IL-33, CRP, IL-33, MCP-1, and MIP-1-beta in monocytes, macrophages, T cells, dendritic cells, neutrophils, and/or microglia; modulated expression of one or more proteins selected from C1qa, C1qB, C1qC, C1s, CIR, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, CD14, CD16, HLA-DR, and CCR2; inhibition of extracellular signal-regulated kinase (ERK) phosphorylation; decreasing tyrosine phosphorylation on multiple cellular proteins; modulated expression of C—C chemokine receptor 7 (CCR7); inhibition of microglial cell chemotaxis toward CCL19 and CCL21 expressing cells; activation of phosphoinositide 3-kinase; reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; inhibition of osteoclast production, decreased rate of osteoclastogenesis, or both; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting maturation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; increasing cell death and apoptosis of monocytes, macrophages, T cells, dendritic cells, neutrophils, and/or microglia; reducing phagocytic activity of monocytes, macrophages, T cells, dendritic cells, neutrophils, and/or microglia; reducing proliferation of monocytes, macrophages, T cells, dendritic cells, neutrophils, and/or microglia; reducing the overall functionality of monocytes, macrophages, T cells, dendritic cells, neutrophils, and/or microglia, phosphorylation of an ITAM containing receptor; phosphorylation of a signaling molecules that mediates ITAM signaling; reducing the activation of pattern recognition receptors; reducing the activation of Toll-like receptors; reducing the activation of damage-associated of clearance of cellular and protein debris; interaction between CD33 and one or more of its ligands; interaction between CD33 and a co-receptor such as CD64; reducing one or more types of clearance selected from apoptotic neuron clearance, nerve tissue debris clearance, dysfunctional synapse clearance, non-nerve tissue debris clearance, bacteria or other foreign body clearance, disease-causing protein clearance, and tumor cell clearance; inhibition of phagocytosis of one or more of apoptotic neurons, nerve tissue debris, non-nerve tissue debris, bacteria, other foreign bodies, disease-causing proteins, disease-causing peptides, disease-causing nucleic acid, disease-causing lipids, or tumor cells; inhibition of clearance of a disease-causing nucleic acid, such as the disease-causing nucleic acid is antisense GGCCCC (G2C4) repeat-expansion RNA; activation of clearance of, a disease-causing protein selected from amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury, traumatic brain injury, age related macular degeneration, glaucoma, retinitis pigmentosa, retinal degeneration, and multiple sclerosis; inhibition of beneficial immune response-to different types of inflammatory and infectious disorders selected from lupus, acute and chronic colitis, wound healing, Crohn's disease, inflammatory bowel disease, ulcerative colitis, obesity, malaria, respiratory tract infection, sepsis, eye infection, systemic infection, lupus, arthritis, low bone density, osteoporosis, osteogenesis, osteopetrotic disease, and Paget's disease of bone; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; promotion of immunosuppressor dendritic cells, immunosuppressor macrophages, myeloid-derived suppressor cells, tumor-associated macrophages, or regulatory T cells; inhibition of one or more ITAM motif containing receptors, such as TREM1, TREM2, FcgR, DAP10, and DAP12; inhibition of one or more receptors containing the motif D/Ex0-2YxxL/IX6-8YxxL/I; inhibition of signaling by one or more pattern recognition receptors (PRRs), such as receptors that identify pathogen-associated molecular patterns (PAMPs), and receptors that identify damage-associated molecular patterns (DAMPs); inhibition of signaling by one or more Toll-like receptors; inhibition of the JAK-STAT signaling pathway; inhibition of nuclear factor kappa-light-chain-enhancer of activated B cells (NFκB); inhibition of PLCγ/PKC/calcium mobilization; inhibition of PI3K/Akt, Ras/MAPK signaling; reduced expression of one or more inflammatory receptors, such as CD86, expressed on one or more of microglia, macrophages, dendritic cells, bone marrow-derived dendritic cells, neutrophils, T cells, T helper cells, or cytotoxic T cells; increasing expression of one or more CD33-dependent genes; normalization of disrupted CD33-dependent gene expression; and decreasing expression of one or more ITAM-dependent genes, such as NFAT transcription factors.

### Modulated Expression of Immune-Related Proteins

In some embodiments, anti-CD33 antibodies for use in the methods of the disclosure may modulate expression of PD-L1, PD-L2, B7-H2, B7-H3, CD200R, CD163 and/or CD206 after binding to a CD33 protein expressed in a cell. Modulated (e.g., increased or decreased) expression may include, without limitation, modulation in gene expression, modulation in transcriptional expression, or modulation in protein expression. Any method known in the art for determining gene, transcript (e.g., mRNA), and/or protein expression may be used. For example, Northern blot analysis may be used to determine anti-inflammatory mediator gene expression levels, RT-PCR may be used to determine the level of anti-inflammatory mediator transcription, and Western blot analysis may be used to determine anti-inflammatory mediator protein levels.

As used herein, PD-L1, PD-L2, B7-H2, B7-H3, CD200R, CD163 and/or CD206 may have modulated expression if its expression in one or more cells of a subject treated with an anti-CD33 antibody is modulated (e.g., increased or decreased) as compared to the expression of PD-L1, PD-L2, B7-H2, B7-H3, CD200R, CD163 and/or CD206 expressed in one or more cells of a corresponding subject that is not treated with the antibody. In some embodiments, anti-CD33 antibodies for use in the methods of the disclosure, may modulate PD-L1, PD-L2, B7-H2, B7-H3, CD200R, CD163 and/or CD206 expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200%, for example, as compared to PD-L1, PD-L2, B7-H3, CD200R, CD163 and/or CD206 expression in one or more cells of a corresponding subject that is not treated with the antibody. In other embodiments, anti-CD33 antibodies for use in the methods of the disclosure modulate PD-L1, PD-L2, B7-H2, B7-H3, CD200R, CD163 and/or CD206 expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to PD-L1, PD-L2, B7-H2, B7-H3, CD200R, CD163 and/or CD206 expression in one or more cells of a corresponding subject that is not treated with the antibody. In some embodiments, anti-CD33 antibodies for use in the methods of the disclosure are useful for preventing, lowering the risk of, or treating conditions and/or diseases associated with abnormal levels of PD-L1, PD-L2, B7-H2, B7-H3, CD200R, CD163 and/or CD206.

### Enhancement or Normalization of the Ability of Bone Marrow-Derived Dendritic Cells to Induce Antigen-Specific T Cell Proliferation

In some embodiments, anti-CD33 antibodies for use in the methods of the disclosure may enhance and/or normalize the ability of bone marrow-derived dendritic cells to induce antigen-specific T cell proliferation after binding to a CD33 protein expressed in a cell.

In some embodiments, anti-CD33 antibodies for use in the methods of the disclosure may enhance and/or normalize the ability of bone marrow-derived dendritic cells to induce antigen-specific T cell proliferation in one or more bone marrow-derived dendritic cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the ability of bone marrow-derived dendritic cells to induce antigen-specific T cell proliferation in one or more bone marrow-derived dendritic cells of a corresponding subject that is not treated with the antibody. In other embodiments, an anti-CD33 antibody may enhance and/or normalize the ability of bone marrow-derived dendritic cells to induce antigen-specific T cell proliferation in one or more bone marrow-derived dendritic cells of a subject by at least at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the ability of bone marrow-derived dendritic cells to induce antigen-specific T cell proliferation in one or more bone marrow-derived dendritic cells of a corresponding subject that is not treated with the antibody. In some embodiments, anti-CD33 antibodies for use in the methods of the disclosure may be beneficial for preventing, lowering the risk of, or treating conditions and/or diseases associated with decreased or dysregulated ability of bone marrow-derived dendritic cells to induce antigen-specific T cell proliferation.

### Proliferation and Survival of CD33-Expressing Cells

In some embodiments, anti-CD33 antibodies for use in the methods of the disclosure may increase the proliferation, survival, and/or function of dendritic cells, macrophages, monocytes, neutrophils, osteoclasts, Langerhans cells of skin, Kupffer cells, T cells, T helper cells, cytotoxic T cells, and microglial cells after binding to CD33 protein expressed on a cell.

Microglial cells are a type of glial cell that are the resident macrophages of the brain and spinal cord, and thus act as the first and main form of active immune defense in the central nervous system (CNS). Microglial cells constitute 20% of the total glial cell population within the brain. Microglial cells are constantly scavenging the CNS for plaques, damaged neurons and infectious agents. The brain and spinal cord are considered “immune privileged” organs in that they are separated from the rest of the body by a series of endothelial cells known as the blood-brain barrier, which prevents most pathogens from reaching the vulnerable nervous tissue. In the case where infectious agents are directly introduced to the brain or cross the blood-brain barrier, microglial cells must react quickly to limit inflammation and destroy the infectious agents before they damage the sensitive neural tissue. Due to the unavailability of antibodies from the rest of the body (few antibodies are small enough to cross the blood brain barrier), microglia must be able to recognize foreign bodies, swallow them, and act as antigen-presenting cells activating T cells. Since this process must be done quickly to prevent potentially fatal damage, microglial cells are extremely sensitive to even small pathological changes in the CNS. They achieve this sensitivity in part by having unique potassium channels that respond to even small changes in extracellular potassium.

As used herein, macrophages of the present disclosure include, without limitation, M1 macrophages, activated M1 macrophages, and M2 macrophages. As used herein, microglial cells of the present disclosure include, without limitation, M1 microglial cells, activated M1 microglial cells, and M2 microglial cells.

In some embodiments, anti-CD33 antibodies for use in the methods of the disclosure may increase the expression of CD80, CD83 and/or CD86 on dendritic cells, monocytes, and/or macrophages.

As used herein, the rate of proliferation, survival, and/or function of macrophages, dendritic cells, monocytes, T cells, neutrophils, osteoclasts, Langerhans cells of skin, Kupffer cells, and/or microglia may be increased if the rate of proliferation, survival, and/or function of macrophages, dendritic cells, monocytes, T cells, neutrophils, osteoclasts, Langerhans cells of skin, Kupffer cells, and/or microglia in a subject treated with an anti-CD33 antibody is greater than the rate of proliferation, survival, and/or function of macrophages, dendritic cells, monocytes, T cells, neutrophils, osteoclasts, Langerhans cells of skin, Kupffer cells, and/or microglia in a corresponding subject that is not treated with the antibody. In some embodiments, anti-CD33 antibodies for use in the methods of the disclosure may increase the rate of proliferation, survival, and/or function of dendritic cells, macrophages, monocytes, osteoclasts, Langerhans cells of skin, Kupffer cells, T cells, and/or microglia in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the rate of proliferation, survival, and/or function of dendritic cells, macrophages, monocytes, osteoclasts, Langerhans cells of skin, Kupffer cells, T cells, and/or microglia in a corresponding subject that is not treated with the antibody. In other embodiments, anti-CD33 antibodies for use in the methods of the disclosure may increase the rate of proliferation, survival, and/or function of dendritic cells, macrophages, monocytes, osteoclasts, Langerhans cells of skin, Kupffer cells, T cells, and/or microglia in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the rate of proliferation, survival, and/or function of dendritic cells, macrophages, monocytes, osteoclasts, Langerhans cells of skin, Kupffer cells, T cells, and/or microglia in a corresponding subject that is not treated with the antibody. In some embodiments, anti-CD33 antibodies for use in the methods of the disclosure may be beneficial for preventing, lowering the risk of, or treating conditions and/or diseases associated with a reduction in proliferation, survival, increased apoptosis and/or function of dendritic cells, neutrophils, macrophages, monocytes, osteoclasts, Langerhans cells of skin, Kupffer cells, T cells, and/or microglia.

### CD33-Dependent Activation of Immune Cells

In some embodiments, anti-CD33 antibodies for use in the methods of the disclosure may increase the activity of cytotoxic T cells helper T cells or both. In some embodiments, anti-CD33 antibodies for use in the methods of the disclosure may be beneficial for preventing, lowering the risk of, or treating conditions and/or diseases associated with decreased activity of cytotoxic T cells helper T cells or both.

In some embodiments, anti-CD33 antibodies for use in the methods of the disclosure may induce an increase in proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells. In some embodiments, anti-CD33 antibodies for use in the methods of the disclosure may induce an increase in proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in the presence of myeloid-derived suppressor cells (MDSC).

As used herein, the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells may include an increased rate if the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject treated with an anti-CD33 antibody is greater than the rate of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody. In some embodiments, anti-CD33 antibodies for use in the methods of the disclosure may increase proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 110%, at least 115%, at least 120%, at least 125%, at least 130%, at least 135%, at least 140%, at least 145%, at least 150%, at least 160%, at least 170%, at least 180%, at least 190%, or at least 200% for example, as compared to the level of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody. In other embodiments, anti-CD33 antibodies for use in the methods of the disclosure may increase proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to the level of proliferation, survival, activity, and/or number of T cells, cytotoxic T cells, CD3⁺ T cells, helper T cells, dendritic cells, macrophages, monocytes, neutrophils, osteoclasts, Langerhans cells of skin, Kupffer cells, and/or microglial cells in a corresponding subject that is not treated with the antibody.

### (ii) Exemplary Anti-CD33 Antibody-Binding Regions

In some embodiments, anti-CD33 antibodies that may be used in the methods of the present disclosure may bind a conformational epitope. In some embodiments, anti-CD33 antibodies that may be used in the methods of the present disclosure may bind a discontinuous CD33 epitope. In some embodiments, the discontinuous CD33 epitope comprises two or more peptides, three or more peptides, four or more peptides, five or more peptides, six or more peptides, seven or more peptides, eight or more peptides, nine or more peptides, or 10 or more peptides. In some embodiments, anti-CD33 antibodies that may be used in the methods of the present disclosure may bind a CD33 epitope comprising one or more peptides. As disclosed herein, CD33 epitopes may comprise one or more peptides comprising five or more, six or more, seven or more, eight or more, nine or more, 10 or more, 11 or more, 12 or more, 13 or more 14 or more, 15 or more, 16 or more, 17 or more, 18 or more, 19 or more, or 20 or more, amino acid residues of the amino acid sequence of SEQ ID NO: 25, or five or more, six or more, seven or more, eight or more, nine or more, 10 or more, 11 or more, 12 or more, 13 or more 14 or more, 15 or more, 16 or more, 17 or more, 18 or more, 19 or more, or 20 or more amino acid residues on a mammalian CD33 protein corresponding to the amino acid sequence of SEQ ID NO: 25.

In some embodiments, anti-CD33 antibodies that may be used in the methods of the present disclosure bind to an epitope of human CD33 that is the same as or overlaps with the CD33 epitope bound by an anti-CD33 antibody comprising a heavy chain variable region comprising the amino acid sequence of SEQ ID NO: 23 and a light chain variable region comprising the amino acid sequence of SEQ ID NO: 24. In some embodiments, anti-CD33 antibodies that may be used in the methods of the present disclosure bind essentially the same CD33 epitope bound by an anti-CD33 antibody comprising a heavy chain variable region comprising the amino acid sequence of SEQ ID NO: 23 and a light chain variable region comprising the amino acid sequence of SEQ ID NO: 24.

In some embodiments, anti-CD33 antibodies that may be used in the methods of the present disclosure competitively inhibit binding of an anti-CD33 antibody comprising a heavy chain variable region comprising the amino acid sequence of SEQ ID NO: 23 and a light chain variable region comprising the amino acid sequence of SEQ ID NO: 24. In some embodiments, anti-CD33 antibodies that may be used in the methods of the present disclosure compete with an anti-CD33 antibody comprising a heavy chain variable region comprising the amino acid sequence of SEQ ID NO: 23 and a light chain variable region comprising the amino acid sequence of SEQ ID NO: 24 for binding to CD33.

In some embodiments, anti-CD33 antibodies that may be used in the methods of the present disclosure competitively inhibit binding of at least one antibody selected from any of the antibodies listed in Table E. In some embodiments, anti-CD33 antibodies that may be used in the methods of the present disclosure competitively inhibit binding of at least one antibody selected from AB64.1.2 and 6C7H54, and any combination thereof. In some embodiments, anti-CD33 antibodies that may be used in the methods of the present disclosure compete with one or more antibodies selected from AB64.1.2 and 6C7H54, and any combination thereof, for binding to CD33 when the anti-CD33 antibody reduces the binding of one or more antibodies selected from AB64.1.2 and 6C7H54, and any combination thereof, to CD33 by an amount the ranges from about 50% to 100%, as compared to binding to CD33 in the absence of the anti-CD33 antibody. In some embodiments, anti-CD33 antibodies that may be used in the methods of the present disclosure compete with one or more antibodies selected from AB64.1.2 and 6C7H54, and any combination thereof for binding to CD33 when the anti-CD33 antibody reduces the binding of one or more antibodies selected from AB64.1.2 and 6C7H54, and any combination thereof, to CD33 by at least 50%, at least 55%, by at least 60%, at least 65%, at least 70%, at least 75%, at least 80%, at least 85%, at least 90%, at least 95%, or 100%, as compared to binding to CD33 in the absence of the anti-CD33 antibody. In some embodiments, anti-CD33 antibodies that may be used in the methods of the present disclosure that reduce the binding of one or more antibodies selected from AB64.1.2 and 6C7H54, and any combination thereof, to CD33 by 100% indicates that the anti-CD33 antibody essentially completely blocks the binding of one or more antibodies selected from AB64.1.2 and 6C7H54, and any combination thereof, to CD33. In some embodiments, the anti-CD33 antibody and the one or more antibodies selected from AB64.1.2 and 6C7H54, and any combination thereof are present in an amount that corresponds to a 10:1 ratio, 9:1 ratio, 8:1 ratio, 7:1 ratio, 6:1 ratio, 5:1 ratio, 4:1 ratio, 3:1 ratio, 2:1 ratio, 1:1 ratio, 0.75:1 ratio, 0.5:1 ratio, 0.25:1 ratio, 0.1:1 ratio, 0.075:1 ratio, 0.050:1 ratio, 0.025:1 ratio, 0.01:1 ratio, 0.0075: ratio, 0.0050:1 ratio, 0.0025:1 ratio, 0.001: ratio, 0.00075:1 ratio, 0.00050:1 ratio, 0.00025:1 ratio, 0.0001: ratio, 1:10 ratio, 1:9 ratio, 1:8 ratio, 1:7 ratio, 1:6 ratio, 1:5 ratio, 1:4 ratio, 1:3 ratio, 1:2 ratio, 1:0.75 ratio, 1:0.5 ratio, 1:0.25 ratio, 1:0.1 ratio, 1:0.075 ratio, 1:0.050 ratio, 1:0.025 ratio, 1:0.01 ratio, 1:0.0075 ratio, 1:0.0050 ratio, 1:0.0025 ratio, 1:0.001 ratio, 1:0.00075 ratio, 1:0.00050 ratio, 1:0.00025 ratio, or 1:0.0001 ratio of anti-CD33 antibody to one or more antibodies selected from AB64.1.2 and 6C7H54, and any combination thereof. In some embodiments, the anti-CD33 antibody is present in excess by an amount that ranges from about 1.5-fold to 100-fold, or greater than 100-fold compared to the amount of the one or more antibodies selected from AB64.1.2 and 6C7H54, and any combination thereof. In some embodiments, the anti-CD33 antibody is present in an amount that is about a 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold excess compared to the amount of the one or more antibodies selected from AB64.1.2 and 6C7H54, and any combination thereof.

In some embodiments, anti-CD33 antibodies that may be used in the methods of the present disclosure bind to an epitope of human CD33 that is the same as or overlaps with the CD33 epitope bound by at least one antibody selected from any of the antibodies listed in Table E. In some embodiments, anti-CD33 antibodies that may be used in the methods of the present disclosure bind to an epitope of human CD33 that is the same as or overlaps with the CD33 epitope bound by at least one antibody selected from AB64.1.2 and 6C7H54.

In some embodiments, anti-CD33 antibodies that may be used in the methods of the present disclosure bind essentially the same CD33 epitope bound by at least one antibody selected from any of the antibodies listed in Table E. In some embodiments, anti-CD33 antibodies that may be used in the methods of the present disclosure bind essentially the same CD33 epitope bound by at least one antibody selected from AB64.1.2 and 6C7H54. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some embodiments, anti-CD33 antibodies that may be used in the methods of the present disclosure compete with one or more antibodies selected from AB64.1.2 and 6C7H54, and any combination thereof for binding to CD33.

Any suitable competition assay or CD33 binding assay known in the art, such as BIAcore analysis, ELISA assays, or flow cytometry, may be utilized to determine whether an anti-CD33 antibody competes with one or more antibodies selected from AB64.1.2 and 6C7H54, and any combination thereof for binding to CD33. In an exemplary competition assay, immobilized CD33 or cells expressing CD33 on the cell surface are incubated in a solution comprising a first labeled antibody that binds to CD33 (e.g., human or non-human primate) and a second unlabeled antibody that is being tested for its ability to compete with the first antibody for binding to CD33. The second antibody may be present in a hybridoma supernatant. As a control, immobilized CD33 or cells expressing CD33 is incubated in a solution comprising the first labeled antibody but not the second unlabeled antibody. After incubation under conditions permissive for binding of the first antibody to CD33, excess unbound antibody is removed, and the amount of label associated with immobilized CD33 or cells expressing CD33 is measured. If the amount of label associated with immobilized CD33 or cells expressing CD33 is substantially reduced in the test sample relative to the control sample, then that indicates that the second antibody is competing with the first antibody for binding to CD33. See, Harlow and Lane (1988) Antibodies: A Laboratory Manual ch. 14 (Cold Spring Harbor Laboratory, Cold Spring Harbor, NY).

(iii) Exemplary Anti-CD33 Antibody Light and Heavy Chain Hypervariable Regions

In some aspects, provided herein are anti-CD33 antibodies that may be used in the methods of the present disclosure, e.g., comprising one or more of the heavy chain hypervariable region (HVR) and/or light chain HVR amino acid sequences described below.

In some embodiments, anti-CD33 antibodies of the disclosure comprise a heavy chain variable region comprising one or more (e.g., one or more, two or more, or all three) HVRs selected from HVR-H1, HVR-H2, and HVR-H3 (as shown in Table E). In some embodiments, the heavy chain variable region comprises an HVR-H1, an HVR-H2, and an HVR-H3 (as shown in Table E).

In some embodiments, the heavy chain variable region comprises the HVR-H1, HVR-H2, and HVR-H3 of antibody AB64.1.2 or 6C7H54, and any combination thereof (as shown in Table E).

In some embodiments, anti-CD33 antibodies of the present disclosure comprise a heavy chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H1 amino acid sequence of antibody AB64.1.2 or 6C7H54; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H2 amino acid sequence of antibody AB64.1.2 or 6C7H54; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H3 amino acid sequence of antibody AB64.1.2 or 6C7H54.

In some embodiments, anti-CD33 antibodies of the present disclosure comprise a heavy chain variable region comprising: an HVR-H1 comprising the amino acid sequence GYTFTDYNLH (SEQ ID NO: 1), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 1; an HVR-H2 comprising the amino acid sequence FIYPSNRITG (SEQ ID NO: 2), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 2; and an HVR-H3 comprising the amino acid sequence SDVDYFDY (SEQ ID NO: 3), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 3.

In some embodiments, anti-CD33 antibodies of the present disclosure comprise a heavy chain variable region comprising: an HVR-H1 comprising the amino acid sequence NYEMN (SEQ ID NO: 12), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 12; an HVR-H2 comprising the amino acid sequence EIRLKSNNYVTNYAASVKG (SEQ ID NO: 13), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 13; and an HVR-H3 comprising the amino acid sequence AGYYVPFAY (SEQ ID NO: 14), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 14.

In some embodiments, anti-CD33 antibodies of the present disclosure comprise a light chain variable region comprising one or more (e.g., one or more, two or more, or all three) HVRs selected from HVR-L1, HVR-L2, and HVR-L3 (as shown in Table E). In some embodiments, the light chain variable region comprises an HVR-L1, an HVR-L2, and an HVR-L3 (as shown in Table E).

In some embodiments, the light chain variable region comprises the HVR-L1, HVR-L2, and HVR-L3 of antibody AB64.1.2 or 6C7H54, and any combination thereof (as shown in Table E).

In some embodiments, anti-CD33 antibodies of the present disclosure comprise a light chain variable region, wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L1 amino acid sequence of antibody AB64.1.2 or 6C7H54; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L2 amino acid sequence of antibody AB64.1.2 or 6C7H54; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L3 amino acid sequence of antibody AB64.1.2 or 6C7H54.

In some embodiments, anti-CD33 antibodies of the present disclosure comprise: a light chain variable region comprising: an HVR-L1 comprising the amino acid sequence RASQSVSTSTYSYMH (SEQ ID NO: 4), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 4; an HVR-L2 comprising the amino acid sequence YASNLES (SEQ ID NO: 5), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 5, and an HVR-L3 comprising the amino acid sequence QHSWEIPLT (SEQ ID NO: 6), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 6.

In some embodiments, anti-CD33 antibodies of the present disclosure comprise: a light chain variable region comprising: an HVR-L1 comprising the amino acid sequence TLSSQHSTYTIE (SEQ ID NO: 15), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 15; an HVR-L2 comprising the amino acid sequence LKKEGSHSTGD (SEQ ID NO: 16), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 16, and an HVR-L3 comprising the amino acid sequence GVGHTIKEQFVYV (SEQ ID NO: 17), or an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to the amino acid sequence of SEQ ID NO: 17.

In some embodiments, anti-CD33 antibodies of the present disclosure comprise a heavy chain variable region comprising one or more (e.g., one or more, two or more, or all three) HVRs selected from HVR-H1, HVR-H2, and HVR-H3 (as shown in Table E), and a light chain variable region comprising one or more (e.g., one or more, two or more, or all three) HVRs selected from HVR-L1, HVR-L2, and HVR-L3 (as shown in Table E). In some embodiments, the heavy chain variable region comprises an HVR-H1, an HVR-H2, and an HVR-H3 (as shown in Table E), and the light chain variable region comprises an HVR-L1, an HVR-L2, and an HVR-L3 (as shown in Table E).

In some embodiments, anti-CD33 antibodies of the present disclosure comprise a heavy chain variable region comprising the HVR-H1, HVR-H2, and HVR-H3 of antibody AB64.1.2 or 6C7H54, and any combination thereof (as shown in Table E); and a light chain variable region comprising the HVR-L1, HVR-L2, and HVR-L3 of antibody AB64.1.2 or 6C7H54, and any combination thereof (as shown in Table E). In some embodiments, anti-CD33 antibodies of the present disclosure comprise a heavy chain variable region comprising an HVR-H1, HVR-H2, and HVR-H3 and a light chain variable region comprising an HVR-L1, HVR-L2, and HVR-L3, wherein the antibody comprises the HVR-H1, HVR-H2, HVR-H3, HVR-L1, HVR-L2, and HVR-L3 of antibody AB64.1.2 or 6C7H54 (as shown in Table E).

In some embodiments, anti-CD33 antibodies of the present disclosure comprise a heavy chain variable region and a light chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H1 amino acid sequence of antibody AB64.1.2 or 6C7H54; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H2 amino acid sequence of antibody AB64.1.2 or 6C7H54; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-H3 amino acid sequence of antibody AB64.1.2 or 6C7H54; and wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L1 amino acid sequence of antibody AB64.1.2 or 6C7H54; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L2 amino acid sequence of antibody AB64.1.2 or 6C7H54; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to an HVR-L3 amino acid sequence of antibody AB64.1.2 or 6C7H54.

In some embodiments, anti-CD33 antibodies of the present disclosure comprise: (a) a heavy chain variable region comprising: an HVR-H1 comprising the amino acid sequence GYTFTDYNLH (SEQ ID NO: 1), or an amino acid sequence with a least about 90% homology to the amino acid sequence of SEQ ID NO: 1; an HVR-H2 comprising the amino acid sequence FIYPSNRITG (SEQ ID NO: 2), or an amino acid sequence with a least about 90% homology to the amino acid sequence of SEQ ID NO: 2; and an HVR-H3 comprising the amino acid sequence SDVDYFDY (SEQ ID NO: 3), or an amino acid sequence with a least about 90% homology to the amino acid sequence of SEQ ID NO: 3; and (b) a light chain variable region comprising: an HVR-L1 comprising the amino acid sequence RASQSVSTSTYSYMH (SEQ ID NO: 4), or an amino acid sequence with a least about 90% homology to the amino acid sequence of SEQ ID NO: 4; an HVR-L2 comprising the amino acid sequence YASNLES (SEQ ID NO: 5), or an amino acid sequence with a least about 90% homology to the amino acid sequence of SEQ ID NO: 5, and an HVR-L3 comprising the amino acid sequence QHSWEIPLT (SEQ ID NO: 6), or an amino acid sequence with a least about 90% homology to the amino acid sequence of SEQ ID NO: 6.

In some embodiments, anti-CD33 antibodies of the present disclosure comprise: (a) a heavy chain variable region comprising: an HVR-H1 comprising the amino acid sequence NYEMN (SEQ ID NO: 12), or an amino acid sequence with a least about 90% homology to the amino acid sequence of SEQ ID NO: 12; an HVR-H2 comprising the amino acid sequence EIRLKSNNYVTNYAASVKG (SEQ ID NO: 13), or an amino acid sequence with a least about 90% homology to the amino acid sequence of SEQ ID NO: 13; and an HVR-H3 comprising the amino acid sequence AGYYVPFAY (SEQ ID NO: 14), or an amino acid sequence with a least about 90% homology to the amino acid sequence of SEQ ID NO: 14; and (b) a light chain variable region comprising: an HVR-L1 comprising the amino acid sequence TLSSQHSTYTIE (SEQ ID NO: 15), or an amino acid sequence with a least about 90% homology to the amino acid sequence of SEQ ID NO: 15; an HVR-L2 comprising the amino acid sequence LKKEGSHSTGD (SEQ ID NO: 16), or an amino acid sequence with a least about 90% homology to the amino acid sequence of SEQ ID NO:

16, and an HVR-L3 comprising the amino acid sequence GVGHTIKEQFVYV (SEQ ID NO: 17), or an amino acid sequence with a least about 90% homology to the amino acid sequence of SEQ ID NO: 17.

### (iv) Exemplary Anti-CD33 Antibody Light and Heavy Chain Variable Regions

In some aspects, provided herein are anti-CD33 antibodies that may be used in the methods of the present disclosure, e.g., comprising any of the heavy chain variable region and/or light chain variable region amino acid sequences described below.

In some embodiments, anti-CD33 antibodies of the present disclosure comprise a heavy chain variable region as shown in Table E. In some embodiments, anti-CD33 antibodies of the present disclosure comprise a heavy chain variable region of antibody AB64.1.2 or 6C7H54 (as shown in Table E).

In some embodiments, anti-CD33 antibodies of the present disclosure comprise a heavy chain variable region comprising the amino acid sequence of SEQ ID NO: 7.

In some embodiments, anti-CD33 antibodies of the present disclosure comprise a heavy chain variable region comprising the amino acid sequence of SEQ ID NO: 18.

In some embodiments, anti-CD33 antibodies of the present disclosure comprise a light chain variable region as shown in Table E. In some embodiments, anti-CD33 antibodies of the present disclosure comprise a light chain variable region of antibody AB64.1.2 or 6C7H54 (as shown in Table E).

In some embodiments, anti-CD33 antibodies of the present disclosure comprise a light chain variable region comprising the amino acid sequence of SEQ ID NO: 8.

In some embodiments, anti-CD33 antibodies of the present disclosure comprise a light chain variable region comprising the amino acid sequence of SEQ ID NO: 19.

In some embodiments, anti-CD33 antibodies of the present disclosure comprise a heavy chain variable region comprising an amino acid sequence selected from SEQ ID NOs: 7 or 18 and a light chain variable region comprising an amino acid sequence selected from SEQ ID NOs: 8 or 19.

In some embodiments, the anti-CD33 antibody comprises a heavy chain variable region comprising the amino acid sequence of SEQ ID NO: 7, and a light chain variable region comprising the amino acid sequence of SEQ ID NO: 8. In some embodiments, the anti-CD33 antibody comprises a heavy chain variable region comprising the amino acid sequence of SEQ ID NO: 18, and a light chain variable region comprising the amino acid sequence of SEQ ID NO: 19.

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

In some embodiments, the anti-CD33 antibody is an anti-CD33 monoclonal antibody selected from AB64.1.2 or 6C7H54.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody AB64.1.2 or to the amino acid sequence of SEQ ID NO: 7; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody AB64.1.2 or to the amino acid sequence of SEQ ID NO: 8. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody AB64.1.2 or to the amino acid sequence of SEQ ID NO: 7, wherein the heavy chain variable domain comprises the HVR-H1, HVR-H2, and HVR-H3 amino acid sequences of antibody AB64.1.2. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody AB64.1.2 or to the amino acid sequence of SEQ ID NO: 8, wherein the light chain variable domain comprises the HVR-L1, HVR-L2, and HVR-L3 amino acid sequences of antibody AB64.1.2. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody AB64.1.2 or to the amino acid sequence of SEQ ID NO: 7 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody AB64.1.2 or the amino acid sequence of SEQ ID NO: 7. In certain embodiments, a total of 1 to 5 amino acids have been substituted, inserted and/or deleted in the heavy chain variable domain amino acid sequence of antibody AB64.1.2 or the amino acid sequence of SEQ ID NO: 7. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody AB64.1.2 or of SEQ ID NO: 7, including post-translational modifications of that sequence. In a particular embodiment, the VH comprises one, two or three HVRs selected from: (a) the HVR-H1 amino acid sequence of antibody AB64.1.2, (b) the HVR-H2 amino acid sequence of antibody AB64.1.2, and (c) the HVR-H3 amino acid sequence of antibody AB64.1.2. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody AB64.1.2 or to the amino acid sequence of SEQ ID NO: 8 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody AB64.1.2 or the amino acid sequence of SEQ ID NO: 8. In certain embodiments, a total of 1 to 5 amino acids have been substituted, inserted and/or deleted in the light chain variable domain amino acid sequence of antibody AB64.1.2 or the amino acid sequence of SEQ ID NO: 8. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody AB64.1.2 or of SEQ ID NO: 8, including post-translational modifications of that sequence. In a particular embodiment, the VL comprises one, two or three HVRs selected from: (a) the HVR-L1 amino acid sequence of antibody AB64.1.2, (b) the HVR-L2 amino acid sequence of antibody AB64.1.2, and (c) the HVR-L3 amino acid sequence of antibody AB64.1.2.

In some embodiments, anti-CD33 antibodies of the present disclosure comprise a light chain variable domain and a heavy chain variable domain, wherein the heavy chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody 6C7H54 or to the amino acid sequence of SEQ ID NO: 18; and/or the light chain variable domain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody 6C7H54 or to the amino acid sequence of SEQ ID NO: 19. In some embodiments, anti-CD33 antibodies of the present disclosure comprise a heavy chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody 6C7H54 or to the amino acid sequence of SEQ ID NO: 18, wherein the heavy chain variable domain comprises the HVR-H1, HVR-H2, and HVR-H3 amino acid sequences of antibody 6C7H54. In some embodiments, anti-CD33 antibodies of the present disclosure comprise a light chain variable domain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody 6C7H54 or to the amino acid sequence of SEQ ID NO: 19, wherein the light chain variable domain comprises the HVR-L1, HVR-L2, and HVR-L3 amino acid sequences of antibody 6C7H54. In some embodiments, the anti-CD33 antibody comprises a heavy chain variable domain (VH) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain variable domain amino acid sequence of antibody 6C7H54 or to the amino acid sequence of SEQ ID NO: 18 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain variable domain amino acid sequence of antibody 6C7H54 or the amino acid sequence of SEQ ID NO: 18. In certain embodiments, a total of 1 to 5 amino acids have been substituted, inserted and/or deleted in the heavy chain variable domain amino acid sequence of antibody 6C7H54 or the amino acid sequence of SEQ ID NO: 18. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VH sequence of antibody 6C7H54or of SEQ ID NO: 18, including post-translational modifications of that sequence. In a particular embodiment, the VH comprises one, two or three HVRs selected from: (a) the HVR-H1 amino acid sequence of antibody 6C7H54, (b) the HVR-H2 amino acid sequence of antibody 6C7H54, and (c) the HVR-H3 amino acid sequence of antibody 6C7H54. In some embodiments, anti-CD33 antibodies of the present disclosure comprise a light chain variable domain (VL) sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain variable domain amino acid sequence of antibody 6C7H54 or to the amino acid sequence of SEQ ID NO: 19 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain variable domain amino acid sequence of antibody 6C7H54 or the amino acid sequence of SEQ ID NO: 19. In certain embodiments, a total of 1 to 5 amino acids have been substituted, inserted and/or deleted in the light chain variable domain amino acid sequence of antibody 6C7H54 or the amino acid sequence of SEQ ID NO: 19. In certain embodiments, substitutions, insertions, or deletions occur in regions outside the HVRs (i.e., in the FR regions). In some embodiments, the substitutions, insertions, or deletions occur in in the FR regions. Optionally, the anti-CD33 antibody comprises the VL sequence of antibody 6C7H54 or of SEQ ID NO: 19, including post-translational modifications of that sequence. In a particular embodiment, the VL comprises one, two or three HVRs selected from: (a) the HVR-L1 amino acid sequence of antibody 6C7H54, (b) the HVR-L2 amino acid sequence of antibody 6C7H54, and (c) the HVR-L3 amino acid sequence of antibody 6C7H54.

In some embodiments, the anti-CD33 antibody is anti-CD33 monoclonal antibody AB64.1.2. In some embodiments, the anti-CD33 antibody is an isolated antibody which binds essentially the same CD33 epitope as AB64.1.2. In some embodiments, the anti-CD33 antibody is an isolated antibody comprising the HVR-H1, HVR-H2, and HVR-H3 of the heavy chain variable domain of monoclonal antibody AB64.1.2. In some embodiments, the anti-CD33 antibody is an isolated antibody comprising the HVR-L1, HVR-L2, and HVR-L3 of the light chain variable domain of monoclonal antibody AB64.1.2. In some embodiments, the anti-CD33 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody AB64.1.2.

In some embodiments, the anti-CD33 antibody is anti-CD33 monoclonal antibody 6C7H54. In some embodiments, the anti-CD33 antibody is an isolated antibody which binds essentially the same CD33 epitope as 6C7H54. In some embodiments, the anti-CD33 antibody is an isolated antibody comprising the HVR-H1, HVR-H2, and HVR-H3 of the heavy chain variable domain of monoclonal antibody 6C7H54. In some embodiments, the anti-CD33 antibody is an isolated antibody comprising the HVR-L1, HVR-L2, and HVR-L3 of the light chain variable domain of monoclonal antibody 6C7H54. In some embodiments, the anti-CD33 antibody is an isolated antibody comprising the HVR-H1, HVR-H2, and HVR-H3 of the heavy chain variable domain and the HVR-L1, HVR-L2, and HVR-L3 of the light chain variable domain of monoclonal antibody 6C7H54.

In certain embodiments, the anti-CD33 antibody is an antagonist antibody. In certain embodiments, the anti-CD33 antibody is an agonist antibody or an inert antibody. In some embodiments, anti-CD33 antibodies of the present disclosure are of the IgG class the IgM class, or the IgA class. In some embodiments, anti-CD33 antibodies of the present disclosure are of the IgG class and have an IgG1, IgG2, IgG3, or IgG4 isotype.

### (v) Exemplary Anti-CD33 Antibody Heavy and Light Chains

In some aspects, provided herein are anti-CD33 antibodies that may be used in the methods of the present disclosure, e.g., comprising any of the heavy chain and/or light chain amino acid sequences described below.

In some embodiments, anti-CD33 antibodies of the present disclosure comprise a heavy chain as shown in Table E. In some embodiments, anti-CD33 antibodies of the present disclosure comprise a heavy chain of antibody AB64.1.2 or 6C7H54 (as shown in Table E).

In some embodiments, anti-CD33 antibodies of the present disclosure comprise a heavy chain comprising the amino acid sequence of SEQ ID NOs: 9 or 10.

In some embodiments, anti-CD33 antibodies of the present disclosure comprise a heavy chain comprising the amino acid sequence of SEQ ID NOs: 20 or 21.

In some embodiments, anti-CD33 antibodies of the present disclosure comprise a light chain as shown in Table E. In some embodiments, anti-CD33 antibodies of the present disclosure comprise a light chain of antibody AB64.1.2 or 6C7H54 (as shown in Table E).

In some embodiments, anti-CD33 antibodies of the present disclosure comprise a light chain comprising the amino acid sequence of SEQ ID NO: 11.

In some embodiments, anti-CD33 antibodies of the present disclosure comprise a light chain comprising the amino acid sequence of SEQ ID NO: 22.

In some embodiments, anti-CD33 antibodies of the present disclosure comprise a heavy chain comprising an amino acid sequence selected from SEQ ID NOs: 9, 10, 20, 21 and a light chain comprising an amino acid sequence selected from SEQ ID NOs: 11 or 22.

In some embodiments, the anti-CD33 antibody comprises a heavy chain comprising the amino acid sequence of SEQ ID NOs: 9 or 10, and a light chain comprising the amino acid sequence of SEQ ID NO: 11.

In some embodiments, the anti-CD33 antibody comprises a heavy chain comprising the amino acid sequence of SEQ ID NOs: 20 or 21, and a light chain comprising the amino acid sequence of SEQ ID NO: 22.

Any of the antibodies of the present disclosure may be produced by a cell line. In some embodiments, the cell line may be a mammalian cell line. In certain embodiments, the cell line may be a hybridoma cell line. In other embodiments, the cell line may be a yeast cell line. Any cell line known in the art suitable for antibody production may be used to produce an antibody of the present disclosure. Exemplary cell lines for antibody production are described throughout the present disclosure.

In some embodiments, the anti-CD33 antibody is an anti-CD33 monoclonal antibody selected from AB64.1.2 or 6C7H54.

In some embodiments, anti-CD33 antibodies of the present disclosure comprise a light chain and a heavy chain, wherein the heavy chain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody AB64.1.2 or to the amino acid sequence of SEQ ID NOs: 9 or 10; and/or the light chain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody AB64.1.2 or to the amino acid sequence of SEQ ID NO: 11. In some embodiments, anti-CD33 antibodies of the present disclosure comprise a heavy chain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody AB64.1.2 or to the amino acid sequence of SEQ ID NOs: 9 or 10, wherein the heavy chain comprises the HVR-H1, HVR-H2, and HVR-H3 amino acid sequences of antibody AB64.1.2. In some embodiments, anti-CD33 antibodies of the present disclosure comprise a light chain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody AB64.1.2 or to the amino acid sequence of SEQ ID NO: 11, wherein the light chain comprises the HVR-L1, HVR-L2, and HVR-L3 amino acid sequences of antibody AB64.1.2. In some embodiments, the anti-CD33 antibody comprises a heavy chain sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody AB64.1.2 or to the amino acid sequence of SEQ ID NOs: 9 or 10 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain amino acid sequence of antibody AB64.1.2 or the amino acid sequence of SEQ ID NOs: 9 or 10. In certain embodiments, a total of 1 to 5 amino acids have been substituted, inserted and/or deleted in the heavy chain amino acid sequence of antibody AB64.1.2 or the amino acid sequence of SEQ ID NOs: 9 or 10. In certain embodiments, substitutions, insertions, or deletions occur in regions outside the HVRs. Optionally, the anti-CD33 antibody comprises the heavy chain sequence of antibody AB64.1.2 or of SEQ ID NOs: 9 or 10, including post-translational modifications of that sequence. In a particular embodiment, the heavy chain comprises one, two or three HVRs selected from: (a) the HVR-H1 amino acid sequence of antibody AB64.1.2, (b) the HVR-H2 amino acid sequence of antibody AB64.1.2, and (c) the HVR-H3 amino acid sequence of antibody AB64.1.2. In some embodiments, anti-CD33 antibodies of the present disclosure comprise a light chain sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody AB64.1.2 or to the amino acid sequence of SEQ ID NO: 11 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain amino acid sequence of antibody AB64.1.2 or the amino acid sequence of SEQ ID NO: 11. In certain embodiments, a total of 1 to 5 amino acids have been substituted, inserted and/or deleted in the light chain amino acid sequence of antibody AB64.1.2 or the amino acid sequence of SEQ ID NO: 11. In certain embodiments, substitutions, insertions, or deletions occur in regions outside the HVRs. Optionally, the anti-CD33 antibody comprises the light chain sequence of antibody AB64.1.2 or of SEQ ID NO: 11, including post-translational modifications of that sequence. In a particular embodiment, the light chain comprises one, two or three HVRs selected from: (a) the HVR-L1 amino acid sequence of antibody AB64.1.2, (b) the HVR-L2 amino acid sequence of antibody AB64.1.2, and (c) the HVR-L3 amino acid sequence of antibody AB64.1.2.

In some embodiments, anti-CD33 antibodies of the present disclosure comprise a light chain and a heavy chain, wherein the heavy chain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody 6C7H54 or to the amino acid sequence of SEQ ID NOs: 20 or 21; and/or the light chain comprises an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody 6C7H54 or to the amino acid sequence of SEQ ID NO: 22. In some embodiments, anti-CD33 antibodies of the present disclosure comprise a heavy chain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody 6C7H54 or to the amino acid sequence of SEQ ID NOs: 20 or 21, wherein the heavy chain comprises the HVR-H1, HVR-H2, and HVR-H3 amino acid sequences of antibody 6C7H54. In some embodiments, anti-CD33 antibodies of the present disclosure comprise a light chain comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody 6C7H54 or to the amino acid sequence of SEQ ID NO: 22, wherein the light chain comprises the HVR-L1, HVR-L2, and HVR-L3 amino acid sequences of antibody 6C7H54. In some embodiments, the anti-CD33 antibody comprises a heavy chain sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a heavy chain amino acid sequence of antibody 6C7H54 or to the amino acid sequence of SEQ ID NOs: 20 or 21 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the heavy chain amino acid sequence of antibody 6C7H54 or the amino acid sequence of SEQ ID NOs: 20 or 21. In certain embodiments, a total of 1 to 5 amino acids have been substituted, inserted and/or deleted in the heavy chain amino acid sequence of antibody 6C7H54 or the amino acid sequence of SEQ ID NOs: 20 or 21. In certain embodiments, substitutions, insertions, or deletions occur in regions outside the HVRs. Optionally, the anti-CD33 antibody comprises the heavy chain sequence of antibody 6C7H54 or of SEQ ID NOs: 20 or 21, including post-translational modifications of that sequence. In a particular embodiment, the heavy chain comprises one, two or three HVRs selected from: (a) the HVR-H1 amino acid sequence of antibody 6C7H54, (b) the HVR-H2 amino acid sequence of antibody 6C7H54, and (c) the HVR-H3 amino acid sequence of antibody 6C7H54. In some embodiments, anti-CD33 antibodies of the present disclosure comprise a light chain sequence having at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% homology to a light chain amino acid sequence of antibody 6C7H54 or to the amino acid sequence of SEQ ID NO: 22 and contains substitutions (e.g., conservative substitutions, insertions, or deletions relative to the reference sequence), but the anti-CD33 antibody comprising that sequence retains the ability to bind to CD33. In certain embodiments, a total of 1 to 10 amino acids have been substituted, inserted, and/or deleted in the light chain amino acid sequence of antibody 6C7H54 or the amino acid sequence of SEQ ID NO: 22. In certain embodiments, a total of 1 to 5 amino acids have been substituted, inserted and/or deleted in the light chain amino acid sequence of antibody 6C7H54 or the amino acid sequence of SEQ ID NO: 22. In certain embodiments, substitutions, insertions, or deletions occur in regions outside the HVRs. Optionally, the anti-CD33 antibody comprises the light chain sequence of antibody 6C7H54 or of SEQ ID NO: 22, including post-translational modifications of that sequence. In a particular embodiment, the light chain comprises one, two or three HVRs selected from: (a) the HVR-L1 amino acid sequence of antibody 6C7H54, (b) the HVR-L2 amino acid sequence of antibody 6C7H54, and (c) the HVR-L3 amino acid sequence of antibody 6C7H54.

Additional anti-CD33 antibodies, e.g., antibodies that specifically bind to a CD33 protein of the present disclosure, may be identified, screened, and/or characterized for their physical/chemical properties and/or biological activities by various assays known in the art.

In some embodiments, an anti-CD33 antibody for use in the methods of the disclosure is any of the antibodies described in WO2016201388, WO2019028283, or WO2020047374, each of which is incorporated herein by reference in its entirety. In some embodiments, an anti-CD33 antibody for use in the methods of the disclosure is antibody AB64.1.2 (e.g., AB-64.1.2), e.g., as described in WO2019028283. In some embodiments, an anti-CD33 antibody for use in the methods of the disclosure is antibody 6C7H54, e.g., as described in WO2020047374. In some embodiments, an anti-CD33 antibody for use in the methods of the disclosure may include anti-CD33 antibodies known in the art, such as those described in, for example, U.S. Pat. Nos. 7,342,110, 7,557,189, 8,119,787, 8,337,855, 8,124,069, 5,730,982, WO2012/074097, WO2004/043344, WO1993/020848, WO2012/045752, WO2007/014743, WO2003/093298, WO2011/036183, WO1991/009058, WO2008/058021, WO2011/038301, WO2016/201389, WO2016/201388, Hoyer et al (2008) Am J Clin Pathol, 129:316-323, Rollins-Ravel and Roth (2012) Histopathology 60:933-942, Perez-Olivia et al (2011) Glycobiol 21:757-770, Ferlasso et al (2000) Eur J Immunol 30:827-833, Vitale et al (2001) Proc Natl Acad Sci USA 98:5764-5769, Jandus et al (2011) Biochem Pharmacol 82:323-332, O'Reilly and Paulson (2009) Trends Pharmcol Sci 30:240-248, Jurcic (2012) Curr Hematol Malig Rep 7:65-73, and Ricart (2011) Clin Cancer Res 17:6417-6427, each of which is incorporated herein by reference in its entirety.

### (vi) Antibody Affinities

Anti-CD33 antibodies for use in the methods of the present disclosure may have nanomolar or even picomolar affinities for the target antigen (e.g., human CD33). In certain embodiments, the dissociation constant (K_{D}) of the antibody is from about 0.001 to about 100 nM. In certain embodiments, the Kp of the antibody is about 0.01 to about 10 nM. In certain embodiments, the Kp of the antibody is about 0.202 to about 8.57 nM. In some embodiments, the Kp of the antibody is less than about or equal to about 100 nM, 90 nM, 80 nM, 70 nM, 60 nM, 50 nM, 40 nM, 30 nM, 20 nM, 10 nM, 9.5 nM, 9 nM, 8.5 nM, 8 nM, 7.5 nM, 7 nM, 6.5 nM, 6 nM, 5.5 nM, 5 nM, 4.5 nM, 4 nM, 3.5 nM, 3 nM, 2.5 nM, 2 nM, 1.5 nM, 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, or 0.005 nM. In some embodiments, the Kp of the antibody is less than about 5.22 nM. In some embodiments, the Kp of the antibody is greater than about or equal to about 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM. 20 nM, 30 nM, 40 nM, 50 nM, 60 nM, 70 nM, 80 nM, or 90 nM. That is, the Kp of the antibody can be any of a range of affinities having an upper limit of about 100 nM, 90 nM, 80 nM, 70 nM, 60 nM, 50 nM, 40 nM, 30 nM, 20 nM, 10 nM, 9.5 nM, 9 nM, 8.5 nM, 8 nM, 7.5 nM, 7 nM, 6.5 nM, 6 nM, 5.5 nM, 5 nM, 4.5 nM, 4 nM, 3.5 nM, 3 nM, 2.5 nM, 2 nM, 1.5 nM, 1 nM, 0.9 nM, 0.8 nM, 0.7 nM, 0.6 nM, 0.5 nM, 0.4 nM, 0.3 nM, 0.2 nM, 0.1 nM, 0.05 nM, 0.01 nM, or 0.005 nM, and an independently selected lower limit of about 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.5 nM, 2 nM, 2.5 nM, 3 nM 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM. 20 nM, 30 nM, 40 nM, 50 nM, 60 nM, 70 nM, 80 nM, or 90 nM, wherein the lower limit is less than the upper limit. In some embodiments, the Kp of the antibody is any of about 10 nM, about 9 nM, about 8 nM, about 7 nM, about 6 nM, about 5 nM, about 4 nM, about 3 nM, about 2 nM, about 1 nM, about 900 pM, about 800 pM, about 700 pM, about 600 pM, about 500 pM, about 400 pM, about 300 pM, about 200 pM, or about 100 pM. Various methods of measuring antibody affinity are known in the art, including, for example, using surface plasmon resonance or BioLayer Interferometry. In some embodiments, the Kp for CD33 is determined at a temperature of approximately 25° C. In some embodiments, the Kp for CD33 is determined at a temperature of approximately 4° C. In some embodiments, the Kp is determined using a monovalent antibody (e.g., a Fab) or a full-length antibody in a monovalent form. In some embodiments, the Kp is determined using a bivalent antibody and monomeric recombinant CD33 protein.

(vii) Antibody Preparation

Anti-CD33 antibodies of the present disclosure can encompass polyclonal antibodies, monoclonal antibodies, humanized and chimeric antibodies, human antibodies, antibody fragments (e.g., Fab, Fab′-SH, Fv, scFv, and F(ab′)₂), bispecific and polyspecific antibodies, multivalent antibodies, heteroconjugate antibodies, conjugated antibodies, library derived antibodies, antibodies having modified effector functions, fusion proteins containing an antibody portion, and any other modified configuration of the immunoglobulin molecule that includes an antigen recognition site, such as an epitope having amino acid residues of a CD33 protein of the present disclosure, including glycosylation variants of antibodies, amino acid sequence variants of antibodies, and covalently modified antibodies. The anti-CD33 antibodies may be human, murine, rat, or of any other origin (including chimeric or humanized antibodies).

### Polyclonal Antibodies

Polyclonal antibodies, such as polyclonal anti-CD33 antibodies, are generally raised in animals by multiple subcutaneous (sc) or intraperitoneal (ip) injections of the relevant antigen and an adjuvant. It may be useful to conjugate the relevant antigen (e.g., a purified or recombinant CD33 protein of the present disclosure) to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The animals are immunized against the desired antigen, immunogenic conjugates, or derivatives by combining, e.g., 100 μg (for rabbits) or 5 μg (for mice) of the protein or conjugate with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

### Monoclonal Antibodies

Monoclonal antibodies, such as monoclonal anti-CD33 antibodies, are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal anti-CD33 antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization (e.g., a purified or recombinant CD33 protein of the present disclosure). Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The immunizing agent will typically include the antigenic protein (e.g., a purified or recombinant CD33 protein of the present disclosure) or a fusion variant thereof. Generally peripheral blood lymphocytes (“PBLs”) are used if cells of human origin are desired, while spleen or lymph node cells are used if non-human mammalian sources are desired. The lymphoctyes are then fused with an immortalized cell line using a suitable fusing agent, such as polyethylene glycol, to form a hybridoma cell. Goding, Monoclonal Antibodies: Principles and Practice, Academic Press (1986), pp. 59-103.

Immortalized cell lines are usually transformed mammalian cells, particularly myeloma cells of rodent, bovine or human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors (available from the Salk Institute Cell Distribution Center, San Diego, California USA), as well as SP-2 cells and derivatives thereof (e.g., X63-Ag8-653) (available from the American Type Culture Collection, Manassas, Virginia USA). Human myeloma and mouse-human heteromyeloma cell lines have also been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen (e.g., a CD33 protein of the present disclosure). Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The culture medium in which the hybridoma cells are cultured can be assayed for the presence of monoclonal antibodies directed against the desired antigen (e.g., a CD33 protein of the present disclosure). Preferably, the binding affinity and specificity of the monoclonal antibody can be determined by immunoprecipitation or by an in vitro binding assay, such as radioimmunoassay (RIA) or enzyme-linked assay (ELISA). Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose chromatography, hydroxylapatite chromatography, gel electrophoresis, dialysis, affinity chromatography, and other methods as described above.

Anti-CD33 monoclonal antibodies may also be made by recombinant DNA methods, such as those disclosed in U.S. Pat. No. 4,816,567, and as described above. DNA encoding the monoclonal antibodies is readily isolated and sequenced using conventional procedures (e.g., by using oligonucleotide probes that specifically bind to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opin. Immunol., 5:256-262 (1993) and Pluckthun, Immunol. Rev. 130:151-188 (1992).

In certain embodiments, anti-CD33 antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) described the isolation of murine and human antibodies, respectively, from phage libraries. Subsequent publications describe the production of high affinity (nanomolar (“nM”) range) human antibodies by chain shuffling (Marks et al., Bio/Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies of desired specificity (e.g., those that bind a CD33 protein of the present disclosure).

The DNA encoding antibodies or fragments thereof may also be modified, for example, by substituting the coding sequence for human heavy- and light-chain constant domains in place of the homologous murine sequences (U.S. Pat. No. 4,816,567; Morrison, et al., Proc. Natl Acad. Sci. USA, 81:6851 (1984)), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

The monoclonal antibodies described herein (e.g., anti-CD33 antibodies of the present disclosure or fragments thereof) may be monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art.

Chimeric or hybrid anti-CD33 antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Humanized Antibodies

Anti-CD33 antibodies of the present disclosure or antibody fragments thereof may further include humanized or human antibodies. Humanized forms of non-human (e.g., murine) antibodies are chimeric immunoglobulins, immunoglobulin chains or fragments thereof (such as Fab, Fab′-SH, Fv, scFv, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally will also comprise at least a portion of an immunoglobulin constant region (Fc), typically that of a human immunoglobulin. Jones et al., Nature 321:522-525 (1986); Riechmann et al., Nature 332:323-329 (1988) and Presta, Curr. Opin. Struct. Biol. 2:593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers, Jones et al., Nature 321:522-525 (1986); Riechmann et al., Nature 332:323-327 (1988); Verhoeyen et al., Science 239:1534-1536 (1988), or through substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody. Sims et al., J. Immunol., 151:2296 (1993); Chothia et al., J. Mol. Biol., 196:901 (1987). Another method uses a particular framework derived from the consensus sequence of all human antibodies of a particular subgroup of light or heavy chains. The same framework may be used for several different humanized antibodies. Carter et al., Proc. Nat'l Acad. Sci. USA 89:4285 (1992); Presta et al., J. Immunol. 151:2623 (1993).

Furthermore, it is important that antibodies be humanized with retention of high affinity for the antigen and other favorable biological properties. To achieve this goal, according to a preferred method, humanized antibodies are prepared by a process of analyzing the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen or antigens (e.g., CD33 proteins of the present disclosure), is achieved. In general, the CDR residues are directly and most substantially involved in influencing antigen binding.

Various forms of the humanized anti-CD33 antibody are contemplated. For example, the humanized anti-CD33 antibody may be an antibody fragment, such as an Fab, which is optionally conjugated with one or more cytotoxic agent(s) in order to generate an immunoconjugate. Alternatively, the humanized anti-CD33 antibody may be an intact antibody, such as an intact IgG1 antibody.

### Antibody Fragments

In certain embodiments there are advantages to using anti-CD33 antibody fragments, rather than whole anti-CD33 antibodies. Smaller fragment sizes allow for rapid clearance and better brain penetration.

In some embodiments, anti-CD33 antibodies that may be used in the methods of the present disclosure include antibody fragments that bind to one or more of a CD33 protein of the present disclosure, a naturally occurring variant of a CD33 protein, and a disease variant of a CD33 protein. In some embodiments, the antibody fragment is an Fab, Fab′, Fab′-SH, F(ab′)₂, Fv or scFv fragment.

In some embodiments, the antibody fragment is used in combination with a second CD33 antibody and/or with one or more antibodies that specifically bind a disease-causing protein selected from: amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and any combination thereof; or with one or more antibodies that bind an immunomodulatory protein selected from the group consisting of: CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, CD47, CSF-1 receptor, Siglec-5, Siglec-7, Siglec-9, Siglec-11, phosphatidylserine, and any combination thereof.

In some embodiments, anti-CD33 antibodies that may be used in the methods of the present disclosure may be functional fragments that bind the same epitope as any of the anti-CD33 antibodies of the present disclosure. In some embodiments, the antibody fragments are miniaturized versions of the anti-CD33 antibodies or antibody fragments of the present disclosure that have the same epitope of the corresponding full-length antibody, but have much smaller molecule weight. Such miniaturized anti-CD33 antibody fragments may have better brain penetration ability and a shorter half-life, which is advantageous for imaging and diagnostic utilities (see e.g., Lütje S et al., Bioconjug Chem. 2014 Feb. 19; 25 (2): 335-41; Tavaré R et al., Proc Natl Acad Sci USA. 2014 Jan. 21; 111 (3): 1108-13; and Wiehr S et al., Prostate. 2014 May; 74 (7): 743-55). Accordingly, in some embodiments, anti-CD33 antibody fragments that may be used in the methods of the present disclosure have better brain penetration as compared to their corresponding full-length antibodies and/or have a shorter half-life as compared to their corresponding full-length antibodies.

Various techniques have been developed for the production of antibody fragments. Traditionally, these fragments were derived via proteolytic digestion of intact antibodies (see, e.g., Morimoto et al., J. Biochem. Biophys. Method. 24:107-117 (1992); and Brennan et al., Science 229:81 (1985)). However, these fragments can now be produced directly by recombinant host cells, for example, using nucleic acids encoding anti-CD33 antibodies of the present disclosure. Fab, Fv and scFv antibody fragments can all be expressed in and secreted from E. coli, thus allowing the straightforward production of large amounts of these fragments. Anti-CD33 antibody fragments can also be isolated from the antibody phage libraries as discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Production of Fab and F(ab′)₂ antibody fragments with increased in vivo half-lives are described in U.S. Pat. No. 5,869,046. In other embodiments, the antibody of choice is a single chain Fv fragment (scFv). See WO 93/16185; U.S. Pat. Nos. 5,571,894 and 5,587,458. The anti-CD33 antibody fragment may also be a “linear antibody,” e.g., as described in U.S. Pat. No. 5,641,870. Such linear antibody fragments may be monospecific or bispecific.

### Bispecific and Polyspecific Antibodies

Bispecific antibodies (BsAbs) are antibodies that have binding specificities for at least two different epitopes, including those on the same or another protein (e.g., one or more CD33 proteins of the present disclosure). Alternatively, one part of a BsAb can be armed to bind to the target CD33 antigen, and another can be combined with an arm that binds to a second protein. Such antibodies can be derived from full length antibodies or antibody fragments (e.g., F(ab′)₂ bispecific antibodies).

In some embodiments, anti-CD33 antibodies that may be used in the methods of the present disclosure include bispecific antibodies that bind to one or more domains on a CD33 protein of the present disclosure and a second antigen. Methods of generating bispecific antibodies are well known in the art and described herein. In some embodiments, bispecific antibodies of the present disclosure bind to one or more amino acid residues of a CD33 protein of the present disclosure, such as one or more amino acid residues of human CD33 (SEQ ID NO: 25), or amino acid residues on a CD33 protein corresponding to amino acid residues of SEQ ID NO: 25. In some embodiments, bispecific antibodies of the present disclosure recognize a first antigen and a second antigen. In some embodiments, the first antigen is a CD33 protein or a naturally occurring variant thereof. In some embodiments, the second antigen is also a CD33 protein, or a naturally occurring variant thereof. In some embodiments, the second antigen is an antigen facilitating transport across the blood-brain-barrier (see, e.g., Gabathuler R., Neurobiol. Dis. 37 (2010) 48-57). Such second antigens include, without limitation, transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30 (A), a protein transduction domain, TAT, Syn-B, penetratin, a poly-arginine peptide, Angiopep peptides such as ANG1005 (see, e.g., Gabathuler, 2010), and other cell surface proteins that are enriched on blood-brain barrier endothelial cells (see, e.g., Daneman et al., PLOS One. 2010 Oct. 29; 5 (10): e13741). In some embodiments, the second antigen is a disease-causing protein including, without limitation, amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides. In some embodiments, the second antigen is one or more ligands and/or proteins expressed on immune cells, including without limitation, CD40, OX40, ICOS, CD28, CD137/4-1BB, CD27, GITR, PD-L1, CTLA4, PD-L2, PD-1, B7-H3, B7-H4, HVEM, LIGHT, BTLA, CD38, TIGIT, VISTA, KIR, GAL9, TIM1, TIM3, TIM4, A2AR, LAG3, DR5, CD39, CD70, CD73, TREM1, TREM2, Siglec-5, Siglec-7, Siglec-9, Siglec-11, SirpA, CD47, CSF1-receptor, CD3, and phosphatidylserine. In some embodiments, the second antigen is a protein, lipid, polysaccharide, or glycolipid expressed on one or more tumor cells.

Methods for making bispecific antibodies are known in the art. Traditional production of full length bispecific antibodies is based on the coexpression of two immunoglobulin heavy-chain/light chain pairs, where the two chains have different specificities. Millstein et al., Nature, 305:537-539 (1983). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, C_{H}2, and C_{H}3 regions. It is preferred to have the first heavy-chain constant region (C_{H}1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only half of the bispecific molecules provides for an easy way of separation. This approach is disclosed in WO 94/04690. For further details of generating bispecific antibodies, see, for example, Suresh et al., Methods in Enzymology 121:210 (1986).

According to another approach described in WO 96/27011 or U.S. Pat. No. 5,731,168, the interface between a pair of antibody molecules can be engineered to maximize the percentage of heterodimers which are recovered from recombinant cell culture. The preferred interface comprises at least a part of the C_{H}3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chains(s) are created on the interface of the second antibody molecule by replacing large amino acid side chains with smaller ones (e.g., alanine or threonine). This provides a mechanism for increasing the yield of the heterodimer over other unwanted end-products such as homodimers.

Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describes the production of fully humanized bispecific antibody F(ab′)₂ molecules. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bivalent antibody fragments directly from recombinant cell culture have also been described. For example, bivalent heterodimers have been produced using leucine zippers. Kostelny et al., J. Immunol., 148 (5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Nat'l Acad. Sci. USA, 90:6444-6448 (1993) has provided an alternative mechanism for making bispecific/bivalent antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

Antibodies with more than two valencies are also contemplated. For example, trispecific antibodies can be prepared. Tutt et al., J. Immunol. 147:60 (1991).

Exemplary bispecific antibodies may bind to two different epitopes on a given molecule (e.g., a CD33 protein of the present disclosure). Alternatively, an arm targeting a CD33 signaling component may be combined with an arm which binds to a triggering molecule on a leukocyte such as a T cell receptor molecule (e.g., CD2, CD3, CD28 or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies may also be used to localize cytotoxic agents to cells which express a particular protein. Such antibodies possess a protein-binding arm and an arm which binds a cytotoxic agent or a radionuclide chelator, such as EOTUBE, DPTA, DOTA or TETA. Another bispecific antibody of interest binds the protein of interest and further binds tissue factor (TF).

### Multivalent Antibodies

A multivalent antibody may be internalized (and/or catabolized) faster than a bivalent antibody by a cell expressing an antigen to which the antibodies bind. The anti-CD33 antibodies of the present disclosure or antibody fragments thereof can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises an Fc region or a hinge region. In this scenario, the antibody will comprise an Fc region and three or more antigen binding sites amino-terminal to the Fc region. The preferred multivalent antibody herein contains three to about eight, but preferably four, antigen binding sites. The multivalent antibody contains at least one polypeptide chain (and preferably two polypeptide chains), wherein the polypeptide chain or chains comprise two or more variable domains. For instance, the polypeptide chain or chains may comprise VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain. The multivalent antibodies may recognize the CD33 antigen as well as, without limitation, additional antigens A beta peptide, antigen or an alpha synuclain protein antigen or, Tau protein antigen or, TDP-43 protein antigen or, prion protein antigen or, huntingtin protein antigen, or RAN, translation Products antigen, including the DiPeptide Repeats, (DPRs peptides) composed of glycine-alanine (GA), glycine-proline (GP), glycine-arginine (GR), proline-alanine (PA), or proline-arginine (PR), insulin receptor, insulin like growth factor receptor, transferrin receptor, or any other antigen that facilitates antibody transfer across the blood brain barrier.

### Heteroconjugate Antibodies

Heteroconjugate antibodies are also within the scope of the present disclosure. Heteroconjugate antibodies are composed of two covalently joined antibodies (e.g., anti-CD33 antibodies of the present disclosure or antibody fragments thereof). For example, one of the antibodies in the heteroconjugate can be coupled to avidin, the other to biotin. Such antibodies have, for example, been proposed to target immune system cells to unwanted cells, U.S. Pat. No. 4,676,980, and have been used to treat HIV infection. International Publication Nos. WO 91/00360, WO 92/200373 and EP 0308936. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980. Heteroconjugate antibodies may be made using any convenient cross-linking methods. Suitable cross-linking agents are well known in the art, and are disclosed in U.S. Pat. No. 4,676,980, along with a number of cross-linking techniques.

### Effector Function Engineering

It may also be desirable to modify an anti-CD33 antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII. In some embodiments, the effector function is impaired by removing N-glycosylation of the Fc region (e.g., in the CH 2 domain of IgG) of the antibody. In some embodiments, the effector function is impaired by modifying regions such as 233-236, 297, and/or 327-331 of human IgG as described in PCT WO 99/58572 and Armour et al., Molecular Immunology 40:585-593 (2003); Reddy et al., J. Immunology 164:1925-1933 (2000).

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### Other Amino Acid Sequence Modifications

Amino acid sequence modifications of anti-CD33 antibodies of the present disclosure, or antibody fragments thereof, are also contemplated. For example, it may be desirable to improve the binding affinity and/or other biological properties of the antibodies or antibody fragments. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics (i.e., the ability to bind or physically interact with a CD33 protein of the present disclosure). The amino acid changes also may alter post-translational processes of the antibody, such as changing the number or position of glycosylation sites.

A useful method for identification of certain residues or regions of the anti-CD33 antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with the target antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, alanine scanning or random mutagenesis is conducted at the target codon or region and the expressed antibody variants are screened for the desired activity.

Amino acid sequence insertions include amino-(“N”) and/or carboxy-(“C”) terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in the Table A below under the heading of “preferred substitutions”. If such substitutions result in a change in biological activity, then more substantial changes, denominated “exemplary substitutions” in Table A, or as further described below in reference to amino acid classes, may be introduced and the products screened.

TABLE A
Amino acid substitutions

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; | leu |
| | norleucine | |
| Leu (L) | norleucine; ile; val; met; | ile |
| | ala; phe | |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | Ser | ser |
| Trp (W) | tyr; phe | tyr |
| Tyr (Y) | trp; phe; thr; ser | phe |
| Val (V) | ile; leu; met; phe; ala; | leu |
| | norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions entail exchanging a member of one of these classes for another class.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

A particularly preferred type of substitutional variant involves substituting one or more hypervariable region residues of a parent antibody (e.g. a humanized or human anti-CD33 antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and the antigen (e.g., a CD33 protein of the present disclosure). Such contact residues and neighboring residues are candidates for substitution according to the techniques elaborated herein. Once such variants are generated, the panel of variants is subjected to screening as described herein and antibodies with superior properties in one or more relevant assays may be selected for further development. Affinity maturation may also be performed by employing a yeast presentation technology such as that disclosed in, for example, WO2009/036379A2; WO2010105256; WO2012009568; and Xu et al., Protein Eng. Des. Sel., 26 (10): 663-70 (2013).

Another type of amino acid variant of the antibody alters the original glycosylation pattern of the antibody. By altering is meant deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of the anti-IgE antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibodies (e.g., anti-CD33 antibodies of the present disclosure) or antibody fragments.

### Antibody Conjugates

Anti-CD33 antibodies of the present disclosure, or antibody fragments thereof, can be conjugated to a detectable marker, a toxin, or a therapeutic agent. Any suitable method known in the art for conjugating molecules, such as a detectable marker, a toxin, or a therapeutic agent to antibodies may be used.

For example, drug conjugation involves coupling of a biological active cytotoxic (anticancer) payload or drug to an antibody that specifically targets a certain tumor marker (e.g. a protein that, ideally, is only to be found in or on tumor cells). Antibodies track these proteins down in the body and attach themselves to the surface of cancer cells. The biochemical reaction between the antibody and the target protein (antigen) triggers a signal in the tumor cell, which then absorbs or internalizes the antibody together with the cytotoxin. After the ADC is internalized, the cytotoxic drug is released and kills the cancer. Due to this targeting, ideally the drug has lower side effects and gives a wider therapeutic window than other chemotherapeutic agents. Technics to conjugate antibodies are disclosed are known in the art (see, e.g., Jane de Lartigue, OncLive Jul. 5, 2012; ADC Review on antibody-drug conjugates; and Ducry et al., (2010). Bioconjugate Chemistry 21 (1): 5-13).

In some embodiments, an anti-CD33 antibody of the present disclosure may be conjugated to a toxin selected from ricin, ricin A-chain, doxorubicin, daunorubicin, a maytansinoid, taxol, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, and a cisplatin.

### Other Antibody Modifications

Anti-CD33 antibodies of the present disclosure, or antibody fragments thereof, can be further modified to contain additional non-proteinaceous moieties that are known in the art and readily available. Preferably, the moieties suitable for derivatization of the antibody are water-soluble polymers. Non-limiting examples of water-soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly (n-vinyl pyrrolidone) polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc. Such techniques and other suitable formulations are disclosed in Remington: The Science and Practice of Pharmacy, 20th Ed., Alfonso Gennaro, Ed., Philadelphia College of Pharmacy and Science (2000).

### Binding Assays and Other Assays

Anti-CD33 antibodies for use in the methods of the present disclosure may be tested for antigen binding activity, e.g., by known methods such as ELISA, surface plasmon resonance (SPR), Western blot, etc.

In some embodiments, competition assays may be used to identify an antibody that competes with any of the antibodies described herein. In some embodiments, competition assays may be used to identify an antibody that competes with any of the antibodies listed in Table E, or selected from AB64.1.2 or 6C7H54 for binding to CD33. In certain embodiments, such a competing antibody binds to the same epitope (e.g., a linear or a conformational epitope) that is bound by any of the antibodies listed in Table E, or selected from AB64.1.2 or 6C7H54. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized CD33 or cells expressing CD33 on a cell surface are incubated in a solution comprising a first labeled antibody that binds to CD33 (e.g., human or non-human primate) and a second unlabeled antibody that is being tested for its ability to compete with the first antibody for binding to CD33. The second antibody may be present in a hybridoma supernatant. As a control, immobilized CD33 or cells expressing CD33 is incubated in a solution comprising the first labeled antibody but not the second unlabeled antibody. After incubation under conditions permissive for binding of the first antibody to CD33, excess unbound antibody is removed, and the amount of label associated with immobilized CD33 or cells expressing CD33 is measured. If the amount of label associated with immobilized CD33 or cells expressing CD33 is substantially reduced in the test sample relative to the control sample, then that indicates that the second antibody is competing with the first antibody for binding to CD33. See, Harlow and Lane (1988) Antibodies: A Laboratory Manual ch. 14 (Cold Spring Harbor Laboratory, Cold Spring Harbor, NY).

In some embodiments, an isolated anti-CD33 antibody of the present disclosure is a human antibody, a humanized antibody, a murine antibody, a bispecific antibody, a monoclonal antibody, a multivalent antibody, or a chimeric antibody. Exemplary descriptions of such antibodies are found throughout the present disclosure.

In some embodiments, anti-CD33 antibodies of the present disclosure bind to a human CD33, or a homolog thereof, including without limitation, a mammalian CD33 protein. In some embodiments, anti-CD33 antibodies of the present disclosure specifically bind to human CD33. In some embodiments, anti-CD33 antibodies of the present disclosure bind to human CD33 and are not cross-reactive with CD33 orthologs or homologs from other species.

In some embodiments, anti-CD33 antibodies of the present disclosure bind to a CD33 protein of the present disclosure expressed on the surface of a cell and modulate (e.g., induce or inhibit) one or more CD33 activities of the present disclosure after binding to the surface-expressed CD33 protein. In some embodiments, anti-CD33 antibodies of the present disclosure are inert antibodies.

(viii) Anti-CD33 Antibodies Capable of Binding Fc Gamma Receptors

In some embodiments, anti-CD33 antibodies that may be used in the methods of the disclosure retain the ability to bind Fc gamma receptors. In some embodiments, such antibodies when they have the correct epitope specificity that is compatible with receptor activation may have features that enable them to cluster and transiently stimulate, for example, the CD33 receptor. In some embodiments, such antibodies may subsequently act as longer-term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33.

In vivo, anti-CD33 antibodies that may be used in the methods of the disclosure may cluster receptors and transiently activate CD33 by any one or more of multiple potential mechanisms. Some isotypes of human antibodies, such as IgG2, have due to their unique structure, an intrinsic ability to cluster receptors, or retain receptors in a clustered configuration, thereby transiently activating receptors such as CD33 without binding to an Fc receptor (e.g., White et al., (2015) Cancer Cell 27, 138-148).

In some embodiments, other antibodies may cluster receptors (e.g., CD33) by binding to Fcg receptors on adjacent cells. In some embodiments, binding of the constant IgG Fc region of the antibody to Fcg receptors may lead to aggregation of the antibodies, and the antibodies in turn may aggregate the receptors to which they bind through their variable region (Chu et al (2008) Mol Immunol, 45:3926-3933; and Wilson et al., (2011) Cancer Cell 19, 101-113). In some embodiments, binding to the inhibitory Fcg receptor FcgR (FcgRIIB) that does not elicit cytokine secretion, oxidative burst, increased phagocytosis, and enhanced antibody-dependent, cell-mediated cytotoxicity (ADCC) is a preferred way to cluster antibodies in vivo, since binding to FcgRIIB is not associated with adverse immune response effects.

There are other mechanisms by which anti-CD33 antibodies that may be used in the methods of the disclosure can cluster receptors. For example, antibody fragments (e.g., Fab fragments) that are cross-linked together may be used to cluster receptors (e.g., CD33) in a manner similar to antibodies with Fc regions that bind Fcg receptors, as described above. In some embodiments, cross-linked antibody fragments (e.g., Fab fragments) may transiently function as agonist antibodies if they induce receptor clustering on the cell surface and bind an appropriate epitope on the target (e.g., CD33).

Therefore, in some embodiments, anti-CD33 antibodies that may be used in the methods of the disclosure may include antibodies that due to their epitope specificity bind CD33 and transiently activate one or more CD33 activities before they, for example, decrease cellular levels of CD33, inhibit one or more CD33 activities, and/or inhibit interaction (e.g., binding) between CD33 and one or more CD33 ligands. In some embodiments, such antibodies may bind to the ligand-binding site on CD33 and transiently mimic the action of a natural ligand, or stimulate the target antigen to transduce signal by binding to one or more domains that are not the ligand-binding sites. In some embodiments, such antibodies would not interfere with ligand binding. In some embodiments, regardless of whether antibodies bind or do not bind to the ligand-binding site on CD33, the antibodies may subsequently act as longer term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33.

In some embodiments, anti-CD33 antibodies that may be used in the methods of the disclosure transiently induce one or more activities of a CD33 protein. In some embodiments, the antibody transiently induces the one or more activities after binding to a CD33 protein that is expressed in a cell. In some embodiments, the CD33 protein is expressed on a cell surface. In some embodiments, the one or more activities of a CD33 protein that are transiently induced by anti-CD33 antibodies of the present disclosure may include, without limitation, phosphorylation of Tyr-340 and Tyr-358 by a Src family tyrosine kinase, such as LCK and FYN; recruitment of and binding to the tyrosine-specific protein phosphatases SHP1 and SHP2; recruitment of and binding to PLC-gamma1, which acts as a guanine nucleotide exchange factor for Dynamini-1; recruitment of and binding to SH2-domain containing protein (e.g., Crk1); recruitment of and binding to the spleen tyrosine kinase Syk; recruitment of and binding to SH3-SH2-SH3 growth factor receptor-bound protein 2 (Grb2); recruitment of and binding to multiple SH2-containing proteins; phosphorylation of Ser-307 and Ser-342 by protein kinase C; modulated expression of one or more anti-inflammatory cytokines, IL-4, IL-10, IL-13, IL-35, IL-16, TGF-beta, IL-1Ra, G-CSF, and soluble receptors for TNF, IFN-beta1a, IFN-beta1b, or IL-6 in monocytes, macrophages, T cells, dendritic cells neutrophils, and/or microglia; decreasing intracellular calcium mobilization; modulated expression of one or more pro-inflammatory cytokines IFN-α4, IFN-b, IL-1B, TNF-α, IL-6, IL-8, CRP, IL-20 family members, LIF, IFN-gamma, OSM, CNTF, GM-CSF, IL-11, IL-12, IL-17, IL-18, IL-23, CXCL10, IL-33, CRP, IL-33, MCP-1, and MIP-1-beta in monocytes, macrophages, T cells, dendritic cells, neutrophils, and/or microglia; modulated expression of one or more proteins selected from C1qa, C1qB, C1qC, C1s, CIR, C4, C2, C3, ITGB2, HMOX1, LAT2, CASP1, CSTA, VSIG4, MS4A4A, C3AR1, GPX1, TyroBP, ALOX5AP, ITGAM, SLC7A7, CD4, ITGAX, PYCARD, CD14, CD16, HLA-DR, and CCR2; inhibition of extracellular signal-regulated kinase (ERK) phosphorylation; decreasing tyrosine phosphorylation on multiple cellular proteins; modulated expression of C—C chemokine receptor 7 (CCR7); inhibition of microglial cell chemotaxis toward CCL19 and CCL21 expressing cells; activation of phosphoinositide 3-kinase; reducing cell growth of monocytes, macrophages, T cells, dendritic cells and/or microglia; reducing T cell proliferation induced by dendritic cells, bone marrow-derived dendritic cells, monocytes, microglia, M1 microglia, activated M1 microglia, M2 microglia, macrophages, M1 macrophages, activated M1 macrophages, and/or M2 macrophages; inhibition of osteoclast production, decreased rate of osteoclastogenesis, or both; decreasing survival of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing proliferation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting migration of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; decreasing one or more functions of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; inhibiting maturation of neutrophils, dendritic cells, bone marrow-derived dendritic cells, macrophages, M1 macrophages, activated M1 macrophages, M2 macrophages, monocytes, osteoclasts, T cells, T helper cells, cytotoxic T cells, granulocytes, microglia, M1 microglia, activated M1 microglia, and/or M2 microglia; increasing cell death and apoptosis of monocytes, macrophages, T cells, dendritic cells, neutrophils, and/or microglia; reducing phagocytic activity of monocytes, macrophages, T cells, dendritic cells, neutrophils, and/or microglia; reducing proliferation of monocytes, macrophages, T cells, dendritic cells, neutrophils, and/or microglia; reducing the overall functionality of monocytes, macrophages, T cells, dendritic cells, neutrophils, and/or microglia, phosphorylation of an ITAM containing receptor; phosphorylation of a signaling molecules that mediates ITAM signaling; reducing the activation of pattern recognition receptors; reducing the activation of Toll-like receptors; reducing the activation of damage-associated of clearance of cellular and protein debris; interaction between CD33 and one or more of its ligands; interaction between CD33 and a co-receptor such as CD64; reducing one or more types of clearance selected from apoptotic neuron clearance, dysfunctional synapse clearance, nerve tissue debris clearance, non-nerve tissue debris clearance, bacteria or other foreign body clearance, disease-causing protein clearance, and tumor cell clearance; inhibition of phagocytosis of one or more of apoptotic neurons, nerve tissue debris, non-nerve tissue debris, bacteria, other foreign bodies, disease-causing proteins, disease-causing peptides, disease-causing nucleic acid, disease-causing lipids, or tumor cells; inhibition of clearance of a disease-causing nucleic acid, such as the disease-causing nucleic acid is antisense GGCCCC (G2C4) repeat-expansion RNA; activation of clearance of, a disease-causing protein selected from amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; inhibition of beneficial immune response to different types of cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, acute myeloid leukemia, pancreatic cancer, prostate cancer, ovarian cancer, fibrosarcoma, and thyroid cancer; inhibition of beneficial immune response to different types of neurological disorders selected from dementia, frontotemporal dementia, Alzheimer's disease, vascular dementia, mixed dementia, Creutzfeldt-Jakob disease, normal pressure hydrocephalus, amyotrophic lateral sclerosis, Huntington's disease, taupathy disease, Nasu-Hakola disease, stroke, acute trauma, chronic trauma, essential tremor, Behcet's disease, Parkinson's disease, dementia with Lewy bodies, multiple system atrophy, Shy-Drager syndrome, progressive supranuclear palsy, cortical basal ganglionic degeneration, acute disseminated encephalomyelitis, granulomartous disorders, Sarcoidosis, diseases of aging, seizures, spinal cord injury,-traumatic brain injury, age related macular degeneration, glaucoma, retinitis pigmentosa, retinal degeneration, and multiple sclerosis; inhibition of beneficial immune response-to different types of inflammatory and infectious disorders selected from lupus, acute and chronic colitis, wound healing, Crohn's disease, inflammatory bowel disease, ulcerative colitis, obesity, malaria, respiratory tract infection, sepsis, eye infection, systemic infection, lupus, arthritis, low bone density, osteoporosis, osteogenesis, osteopetrotic disease, and Paget's disease of bone; inhibition of phagocytosis of one or more of apoptotic neurons, nerve tissue debris, dysfunctional synapses, non-nerve tissue debris, bacteria, other foreign bodies, disease-causing proteins, disease-causing peptides, disease-causing nucleic acids, or tumor cells, where the disease-causing nucleic acids may be an antisense GGCCCC (G2C4) repeat-expansion RNA, the disease-causing proteins may include amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides, and the tumor cells may be from a cancer selected from bladder cancer, brain cancer, breast cancer, colon cancer, rectal cancer, endometrial cancer, kidney cancer, renal cell cancer, renal pelvis cancer, leukemia, lung cancer, melanoma, non-Hodgkin's lymphoma, pancreatic cancer, prostate cancer, ovarian cancer, fibrosarcoma, or thyroid cancer; binding to CD33 ligand on tumor cells; binding to CD33 ligand on dendritic cells, bone marrow-derived dendritic cells, monocytes, microglia, T cells, neutrophils, and/or macrophages; inhibition of tumor cell killing by one or more of microglia, macrophages, dendritic cells, bone marrow-derived dendritic cells, neutrophils, T cells, T helper cells, or cytotoxic T cells; inhibition of anti-tumor cell proliferation activity of one or more of microglia, macrophages, dendritic cells, bone marrow-derived dendritic cells, neutrophils, T cells, T helper cells, or cytotoxic T cells; inhibition of anti-tumor cell metastasis activity of one or more of microglia, macrophages, dendritic cells, bone marrow-derived dendritic cells, neutrophils, T cells, T helper cells, or cytotoxic T cells; promotion of immunosuppressor dendritic cells, immunosuppressor macrophages, myeloid-derived suppressor cells, tumor-associated macrophages, or regulatory T cells; inhibition of one or more ITAM motif containing receptors, such as TREM1, TREM2, FcgR, DAP10, and DAP12; inhibition of one or more receptors containing the motif D/Ex0-2YxxL/IX6-8YxxL/I; inhibition of signaling by one or more pattern recognition receptors (PRRs), such as receptors that identify pathogen-associated molecular patterns (PAMPs), and receptors that identify damage-associated molecular patterns (DAMPs); inhibition of signaling by one or more Toll-like receptors; inhibition of the JAK-STAT signaling pathway; inhibition of nuclear factor kappa-light-chain-enhancer of activated B cells (NFκB); inhibition of PLCγ/PKC/calcium mobilization; inhibition of PI3K/Akt, Ras/MAPK signaling; modulated expression of one or more inflammatory receptors, such as CD86, expressed on one or more of microglia, macrophages, dendritic cells, bone marrow-derived dendritic cells, neutrophils, T cells, T helper cells, or cytotoxic T cells; increasing expression of one or more CD33-dependent genes; normalization of disrupted CD33-dependent gene expression; and decreasing expression of one or more ITAM-dependent genes, such as NFAT transcription factors. Anti-CD33 antibodies that may be used in the methods of the disclosure may be tested for their ability to transiently induce one or more activities of a CD33 protein utilizing any suitable technique or assay known in the art and disclosed herein. Regardless of the activities that such antibodies transiently induce, such antibodies may subsequently act as longer-term inhibitors of CD33 expression and/or one or more activities of a CD33 protein by inducing CD33 degradation, CD33 desensitization, CD33 cleavage, CD33 internalization, CD33 shedding, downregulation of CD33 expression, and/or lysosomal degradation of CD33. In some embodiments, the CD33 antibody transiently induces one or more activities of a CD33 protein independently of binding to an Fc receptor.

Exemplary antibody Fc isotypes and modifications are provided in Table B below. In some embodiments, anti-CD33 antibodies that may be used in the methods of the disclosure may be capable of binding an Fc gamma receptor and have an Fc isotype listed in Table B below.

TABLE B
Exemplary anti-CD33 antibody Fc isotypes that are
capable of binding Fc gamma receptor

| Fc Isotype | Mutation (EU numbering scheme) |
|---|---|
| IgG1 | N297A |
| IgG1 | D265A and N297A |
| IgG1 | D270A |
| IgG1 | L234A and L235A |
| | L234A and G237A |
| | L234A and L235A and G237A |
| IgG1 | D270A, and/or P238D, and/or L328E, and/or E233D, |
| | and/or G237D and/or H268D, and/or P271G, and/or |
| | A330R |
| IgG1 | P238D and L328E and E233D and G237D and |
| | H268D and P271G and A330R |
| IgG1 | P238D and L328E and G237D and H268D and |
| | P271G and A330R |
| IgG1 | P238D and S267E and L328F and E233D and |
| | G237D and H268D and P271G and A330R |
| IgG1 | P238D and S267E and L328F and G237D and |
| | H268D and P271G and A330R |
| IgG2 | V234A and G237A |
| IgG4 | L235A and G237A and E318A |
| IgG4 | S228P and L236E |
| IgG2/4 hybrid | IgG2 aa 118 to 260 and IgG4 aa 261 to 447 |
| | H268Q and V309L; and A330S and P331S |
| IgG1 | C226S and C229S and E233P and L234V and L235A |
| IgG1 | L234F and L235E and P331S |
| IgG2 | C232S or C233S |
| IgG2 | A330S and P331S |
| IgG1 | S267E and L328F |
| | S267E alone |
| IgG2 | S267E and L328F |
| IgG4 | S267E and L328F |
| IgG2 | WT HC with Kappa (light chain) LC |
| | HC C127S with Kappa LC |
| | Kappa LC C214S |
| | Kappa LC C214S and HC C233S |
| | Kappa LC C214S and HC C232S |
| | Any of the above listed mutations together with P330S |
| | and P331S mutations |
| | F(ab′)2 fragment of WT IgG1 and any of the above listed |
| | mutations |
| IgG1 | Substitute the Constant Heavy 1 (CH1) and hinge region |
| | of IgG1 With CH1 and hinge region of IGg2 |
| | ASTKGPSVFP LAPCSRSTSE STAALGCLVK |
| | DYFPEPVTVS WNSGALTSGV HTFPAVLQSS |
| | GLYSLSSVVT VPSSNFGTQT YTCNVDHKPS |
| | NTKVDKTVER KCCVECPPCP (SEQ ID NO: 26) |
| | With a Kappa LC |
| IgG1 | Any of the above listed mutations together with |
| | A330L/A330S and/or L234F and/or L235E and/or |
| | P331S |
| IgG1, IgG2, or IgG4 | Any of the above listed mutations together with M252Y |
| | and/or S254T and/or T256E |
| Mouse IgG1, mouse | For mouse disease models |
| IgG2a, mouse IgG2b | |
| IgG4 | WT |
| IgG1 | Any of the above listed mutation together with E430G, |
| | E430S, E430F, E430T, E345K, E345Q, E345R, E345Y, |
| | S440Y, S440W and/or any combination thereof. |
| IgG2 | Any of the above listed mutation together with E430G, |
| | E430S, E430F, E430T, E345K, E345Q, E345R, E345Y, |
| | S440Y, S440W and/or any combination thereof. |

In addition, and without wishing to be bound to theory, it is thought that antibodies with human IgG1 or IgG3 isotypes and mutants thereof (e.g. Strohl (2009) Current Opinion in Biotechnology 2009, 20:685-691) that bind the Fog Receptors I, IIA, IIC, IIIA, IIIB in human and/or Fcg Receptors I, III and IV in mouse, may also act as transient agonist antibodies.

In some embodiments, the Fc gamma receptor-binding antibody is of the IgG class, the IgM class, or the IgA class. In some embodiments, the Fc gamma receptor-binding antibody has an IgG1, IgG2, IgG3, or IgG4 isotype. In some embodiments, the antibody comprises one or more (e.g., one or more, two or more, three or more, four or more, five or more, six or more, seven or more, eight or more, nine or more, 10 or more, 11 or more, 12 or more, or all thirteen) amino acid substitutions in the Fc region at a residue position selected from the group consisting of: C127S, L234A, L234F, L235A, L235E, S267E, K322A, L328F, A330S, P331S, E345R, E430G, S440Y in any combination (residue position according to EU or Kabat numbering). In some embodiments, the Fc region comprises an amino acid substitution at position E430G. In some embodiments, the Fc region comprises an amino acid substitution at positions L243A, L235A, and P331A. In some embodiments, the Fc region comprises an amino acid substitution at positions L243A, L235A, P331A. In some embodiments, the Fc region comprises an amino acid substitution at positions K322A and E430G. In some embodiments, the Fc region comprises an amino acid substitution at positions P331S and E430G. In some embodiments, the Fc region comprises an amino acid substitution at positions A330S, P331S, and E430G. In some embodiments, the Fc region comprises an amino acid substitution at positions K322A, A330S, and P331S. In some embodiments, the Fc region comprises an amino acid substitution at positions K322A, P331S, and E430G. In some embodiments, the Fc region comprises an amino acid substitution at positions A330S, P331S, and E430G. In some embodiments, the Fc region comprises an amino acid substitution at positions S267E and L328F. In some embodiments, the Fc region comprises an amino acid substitution at position C127S. In some embodiments, the Fc region comprises an amino acid substitution at positions E345R, E430G and S440Y. In some embodiments, the Fc region comprises an amino acid substitution at positions L243A, L235A, and P331S.

In certain embodiments, the Fc gamma receptor-binding antibody has an IgG2 isotype. In some embodiments, the Fc gamma receptor-binding antibody contains a human IgG2 constant region. In some embodiments, the human IgG2 constant region includes an Fc region. In some embodiments, the Fc gamma receptor-binding antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from V234A (Alegre et al., (1994) Transplantation 57:1537-1543. 31; Xu et al., (2000) Cell Immunol, 200:16-26), G237A (Cole et al. (1999) Transplantation, 68:563-571), H268Q, V309L, A330S, P331S (US 2007/0148167; Armour et al. (1999) Eur J Immunol 29:2613-2624; Armour et al. (2000) The Haematology Journal 1 (Suppl. 1): 27; Armour et al. (2000) The Haematology Journal 1 (Suppl. 1): 27), C232S, and/or C233S (White et al. (2015) Cancer Cell 27, 138-148), S267E, L328F (Chu et al., (2008) Mol Immunol, 45:3926-3933), M252Y, S254T, and/or T256E, where the amino acid position is according to the EU or Kabat numbering convention.

In some embodiments, the Fc gamma receptor-binding antibody has an IgG2 isotype with a heavy chain constant domain that contains a C127S amino acid substitution, where the amino acid position is according to the EU or Kabat numbering convention (White et al., (2015) Cancer Cell 27, 138-148; Lightle et al., (2010) PROTEIN SCIENCE 19:753-762; and WO2008079246).

In some embodiments, the Fc gamma receptor-binding antibody has an IgG2 isotype with a Kappa light chain constant domain that contains a C214S amino acid substitution, where the amino acid position is according to the EU or Kabat numbering convention (White et al., (2015) Cancer Cell 27, 138-148; Lightle et al., (2010) PROTEIN SCIENCE 19:753-762; and WO2008079246).

In certain embodiments, the Fc gamma receptor-binding antibody has an IgG1 isotype. In some embodiments, the Fc gamma receptor-binding antibody contains a mouse IgG1 constant region. In some embodiments, the Fc gamma receptor-binding antibody contains a human IgG1 constant region. In some embodiments, the human IgG1 constant region includes an Fc region. In some embodiments, the Fc gamma receptor-binding antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), D270A, L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), P238D, L328E, E233D, G237D, H268D, P271G, A330R, C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, T256E, N297Q, P238S, P238A, A327Q, A327G, P329A, K322A, and/or T394D, where the amino acid position is according to the EU or Kabat numbering convention.

In some embodiments, the antibody includes an IgG2 isotype heavy chain constant domain 1 (CH1) and hinge region (White et al., (2015) Cancer Cell 27, 138-148). In certain embodiments, the IgG2 isotype CH1 and hinge region contain the amino acid sequence of ASTKGPSVFPLAPCSRSTSESTAALGCLVKDYFPEPVTVSWNSGALTSGVHTFPAVLQSSGLY SLSSVVTVPSSNFGTQTYTCNVDHKPSNTKVDKTVERKCCVECPPCP (SEQ ID NO: 26). In some embodiments, the antibody Fc region contains a S267E amino acid substitution, a L328F amino acid substitution, or both, and/or a N297A or N297Q amino acid substitution, where the amino acid position is according to the EU or Kabat numbering convention.

In certain embodiments, the Fc gamma receptor-binding antibody has an IgG4 isotype. In some embodiments, the Fc gamma receptor-binding antibody contains a human IgG4 constant region. In some embodiments, the human IgG4 constant region includes an Fc region. In some embodiments, the Fc gamma receptor-binding antibody binds an inhibitory Fc receptor. In certain embodiments, the inhibitory Fc receptor is inhibitory Fc-gamma receptor IIB (FcγIIB). In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from L235A, G237A, S228P, L236E (Reddy et al., (2000) J Immunol, 164:1925-1933), S267E, E318A, L328F, M252Y, S254T, and/or T256E, where the amino acid position is according to the EU or Kabat numbering convention.

In certain embodiments, the Fc gamma receptor-binding antibody has a hybrid IgG2/4 isotype. In some embodiments, the Fc gamma receptor-binding antibody includes an amino acid sequence containing amino acids 118 to 260 according to EU or, Kabat numbering of human IgG2 and amino acids 261-447 according to EU or, Kabat numbering of human IgG4 (WO 1997/11971; WO 2007/106585).

In certain embodiments, the antibody contains a mouse IgG4 constant region (Bartholomaeus, et al. (2014). J. Immunol. 192, 2091-2098).

In some embodiments, the Fc region further contains one or more additional amino acid substitutions selected from the group consisting of A330L, L234F; L235E, or P331S according to EU or, Kabat numbering; and any combination thereof.

In certain embodiments, the antibody contains one or more amino acid substitutions in the Fc region at a residue position selected from C127S, L234A, L234F, L235A, L235E, S267E, K322A, L328F, A330S, P331S, E345R, E430G, S440Y, and any combination thereof, where the numbering of the residues is according to EU or Kabat numbering. In some embodiments, the Fc region contains an amino acid substitution at positions E430G, L243A, L235A, and P331S, where the numbering of the residue position is according to EU numbering. In some embodiments, the Fc region contains an amino acid substitution at positions E430G and P331S, where the numbering of the residue position is according to EU numbering. In some embodiments, the Fc region contains an amino acid substitution at positions E430G and K322A, where the numbering of the residue position is according to EU numbering. In some embodiments, the Fc region contains an amino acid substitution at positions E430G, A330S, and P331S, where the numbering of the residue position is according to EU numbering. In some embodiments, the Fc region contains an amino acid substitution at positions E430G, K322A, A330S, and P331S, where the numbering of the residue position is according to EU numbering. In some embodiments, the Fc region contains an amino acid substitution at positions E430G, K322A, and A330S, where the numbering of the residue position is according to EU numbering. In some embodiments, the Fc region contains an amino acid substitution at positions E430G, K322A, and P331S, where the numbering of the residue position is according to EU numbering. In some embodiments, the Fc region contains an amino acid substitution at positions S267E and L328F, where the numbering of the residue position is according to EU numbering. In some embodiments, the Fc region contains an amino acid substitution at position C127S, where the numbering of the residue position is according to EU numbering. In some embodiments, the Fc region contains an amino acid substitution at positions E345R, E430G and S440Y, where the numbering of the residue position is according to EU numbering. In some embodiments, the Fc region comprises an amino acid substitution at positions L243A, L235A, and P331S, wherein the numbering of the residue position is according to EU numbering.

### (ix) Inert Antibodies

Another class of anti-CD33 antibodies that may be used in the methods of the disclosure includes inert antibodies. As used herein, “inert” antibodies refer to antibodies that specifically bind their target antigen (e.g., CD33) but do not modulate (e.g., decrease/inhibit or activate/induce) antigen function. For example, in the case of CD33, inert antibodies do not modulate cellular levels of CD33, do not modulate interaction (e.g., binding) between CD33 and one or more CD33 ligands, or do not modulate one or more activities of a CD33 protein. In some embodiments, antibodies that do not have the ability to cluster CD33 on the cell surface may be inert antibodies even if they have an epitope specificity that is compatible with receptor activation.

In some embodiments, anti-CD33 antibodies that may be used in the methods of the disclosure may include antibodies that bind CD33 but, due to their epitope specificity, or characteristics, do not decrease cellular levels of CD33 and/or inhibit interaction (e.g., binding) between CD33 and one or more CD33 ligands. In some embodiments, such antibodies can be used as cargo to, for example, transport toxins (e.g., chemotherapeutics) into tumor cells. Therefore, in some embodiments, anti-CD33 antibodies that may be used in the methods of the disclosure are inert antibodies that bind CD33 but are incapable of decreasing cellular levels of CD33, inhibiting interaction (e.g., binding) between CD33 and one or more CD33 ligands, or inducing one or more activities of a CD33 protein.

Antibodies that either decrease or do not decrease cellular levels of CD33 on cells can be combined with an inert Fc region that displays reduced binding to one or more Fcg Receptor. Examples of such Fc regions and modifications are provided herein. In some embodiments, the antibody with an inert Fc region has an Fc isotype provided herein.

### (x) Inhibitory Anti-CD33 Antibodies

Another class of anti-CD33 antibodies that may be used in the methods of the disclosure includes antibodies that block or otherwise inhibit one or more CD33 activities. In some embodiments, anti-CD33 antibodies that may be used in the methods of the disclosure may include antibodies that reduce cellular levels of CD33 (e.g., cell surface levels of CD33, intracellular levels of CD33, and/or total levels of CD33), inhibit interaction (e.g., binding) between CD33 and/or one or more CD33 ligands, and inhibit one or more activities of a CD33 protein. Such antibodies inhibit one or more activities of a CD33 protein either by preventing interaction (e.g., binding) between CD33 and one or more CD33 ligands or by preventing signal transduction from the extracellular domain of CD33 into the cell cytoplasm in the presence of one or more CD33 ligands. Antibodies also can inhibit one or more activities of a CD33 protein by decreasing cell surface levels of CD33 by inducing CD33 degradation, CD33 desensitization, CD33 cleavage, CD33 internalization, CD33 shedding, downregulation of CD33 expression, and/or lysosomal degradation of CD33. In some embodiments, such anti-CD33 antibodies may not transiently activate CD33.

In certain embodiments, anti-CD33 antibodies that may be used in the methods of the disclosure decrease cellular levels of CD33, decrease cell surface levels of CD33, decrease intracellular levels of CD33, decrease total levels of CD33, or any combination thereof. In certain embodiments, anti-CD33 antibodies that may be used in the methods of the disclosure decrease cellular levels of CD33, decrease cell surface levels of CD33, decrease intracellular levels of CD33, decrease total levels of CD33, or any combination thereof, in monocytes, granulocytes, peripheral blood monocytes, peripheral blood granulocytes, macrophages, dendritic cells, CSF monocytes, and/or CSF granulocytes.

In certain embodiments, an anti-CD33 antibody that may be used in the methods of the disclosure induces CD33 degradation, CD33 cleavage, CD33 internalization, CD33 downregulation, or any combination thereof. In certain embodiments, the anti-CD33 antibody induces CD33 degradation, CD33 cleavage, CD33 internalization, CD33 downregulation, or any combination thereof, in monocytes, granulocytes, macrophages, dendritic cells, peripheral blood monocytes, peripheral blood granulocytes, CSF monocytes, and/or CSF granulocytes.

In certain embodiments, anti-CD33 antibody that may be used in the methods of the disclosure decreases cellular levels of CD33 and inhibits the interaction between CD33 and a CD33 ligand. In certain embodiments, the anti-CD33 antibody decreases cellular levels of CD33 and inhibits the interaction between CD33 and a CD33 ligand in monocytes, granulocytes, peripheral blood monocytes, macrophages, dendritic cells, peripheral blood granulocytes, CSF monocytes, and/or CSF granulocytes.

In some embodiments, administration of an anti-CD33 antibody according to the methods provided herein reduces the cell surface level of CD33 by at least about 70% (e.g., at least about 70%, at least about 75%, at least about 80%, at least about 85%, at least about 90%, at least about 95%, or at least about 99%) compared to the cell surface level of CD33 prior to administration of the anti-CD33 antibody. In some embodiments, administration of an anti-CD33 antibody according to the methods provided herein reduces the cell surface level of CD33 by at least about 75% compared to the cell surface level of CD33 prior to administration of the anti-CD33 antibody. In some embodiments, administration of an anti-CD33 antibody according to the methods provided herein reduces the cell surface level of CD33 by at least about 80% compared to the cell surface level of CD33 prior to administration of the anti-CD33 antibody. In some embodiments, administration of an anti-CD33 antibody according to the methods provided herein reduces the cell surface level of CD33 by at least about 85% compared to the cell surface level of CD33 prior to administration of the anti-CD33 antibody. In some embodiments, administration of an anti-CD33 antibody according to the methods provided herein reduces the cell surface level of CD33 by at least about 90% compared to the cell surface level of CD33 prior to administration of the anti-CD33 antibody. In some embodiments, the reduction in cell surface levels of CD33 is present for at least about 10 days (e.g., at least 10 days, at least 15 days, at least 20 days, at least 25 days, at least 30 days, at least 35 days, at least 40 days, at least 45 days, at least 50 days, at least 55 days, at least 60 days, at least 70 days, at least 80 days, at least 90 days, or at least 100 days) after administration of the anti-CD33 antibody. In some embodiments, the reduction in cell surface levels of CD33 is present for at least about 12 days after administration of the anti-CD33 antibody. In some embodiments, the reduction in cell surface levels of CD33 is present for at least about 17 days after administration of the anti-CD33 antibody. In some embodiments, the reduction in cell surface levels of CD33 is present for at least about 29 days after administration of the anti-CD33 antibody. In some embodiments, the reduction in cell surface levels of CD33 is present for at least about 42 days after administration of the anti-CD33 antibody. In some embodiments, the reduction in cell surface levels of CD33 is present for at least about 56 days after administration of the anti-CD33 antibody. In some embodiments, the reduction in cell surface levels of CD33 is present for at least about 84 days after administration of the anti-CD33 antibody. In some embodiments, the reduction in cell surface level of CD33 is a reduction in the cell surface level of CD33 on monocytes, granulocytes, macrophages, dendritic cells, peripheral blood monocytes, peripheral blood granulocytes, CSF monocytes, and/or CSF granulocytes of the individual.

In some embodiments, anti-CD33 antibodies that may be used in the methods of the disclosure reduce cell surface levels of CD33 by more than about 70% (e.g., about 70% or more, about 75% or more, about 80% or more, about 85% or more, about 90% or more, about 95% or more, or about 99% or more) compared to the cell surface levels of CD33 prior to administration of the anti-CD33 antibody. In some embodiments, anti-CD33 antibodies that may be used in the methods of the disclosure reduce cell surface levels of CD33 by more than about 70% after a single intravenous dose of between 1.6 mg/kg and about 15 mg/kg (e.g., about 1.6 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg) of the antibody compared to the cell surface levels of CD33 prior to administration of the anti-CD33 antibody. In some embodiments, anti-CD33 antibodies that may be used in the methods of the disclosure reduce cell surface levels of CD33 by more than about 70% after a single intravenous dose of 1.6 mg/kg of the antibody compared to the cell surface levels of CD33 prior to administration of the anti-CD33 antibody. In some embodiments, anti-CD33 antibodies that may be used in the methods of the disclosure reduce cell surface levels of CD33 by more than about 70% after a single intravenous dose of 15 mg/kg of the antibody compared to the cell surface levels of CD33 prior to administration of the anti-CD33 antibody. In some embodiments, anti-CD33 antibodies that may be used in the methods of the disclosure reduce cell surface levels of CD33 by more than about 80% after a single intravenous dose of 15 mg/kg of the antibody compared to the cell surface levels of CD33 prior to administration of the anti-CD33 antibody. In some embodiments, anti-CD33 antibodies that may be used in the methods of the disclosure reduce cell surface levels of CD33 by more than about 85% after a single intravenous dose of 15 mg/kg of the antibody compared to the cell surface levels of CD33 prior to administration of the anti-CD33 antibody. In some embodiments, anti-CD33 antibodies that may be used in the methods of the disclosure reduce cell surface levels of CD33 by more than about 90% after a single intravenous dose of 15 mg/kg of the antibody compared to the cell surface levels of CD33 prior to administration of the anti-CD33 antibody. In some embodiments, the reduction in cell surface levels of CD33 occurs in monocytes from whole blood, granulocytes, peripheral blood monocytes, macrophages, dendritic cells, peripheral blood granulocytes, CSF monocytes, and/or CSF granulocytes from the individual.

In some embodiments, the cell surface level of CD33 may be determined according to any method known in the art. In some embodiments, the cell surface level of CD33 may be determined using flow cytometry. In some embodiments, the cell surface level of CD33 is expressed as Mean Fluorescence Intensity (MFI). In some embodiments, the cell surface level of CD33 is expressed as Molecules of Equivalent Soluble Fluorochrome (MESF) (e.g., see Schawrts et al., (2004) Clin Cytometry 57B: 1-6).

In some embodiments, anti-CD33 antibodies that may be used in the methods of the disclosure may have the epitope specificity of a transient agonist anti-CD33 antibody of the present disclosure, but have an Fc domain that is not capable of binding Fcg receptors and thus is unable to, for example, transiently clustering and activating CD33.

In some embodiments, anti-CD33 antibodies that may be used in the methods of the disclosure have, without limitation, one or more of the following activities: the ability to decrease binding of a CD33 protein to one or more CD33 ligands, such as sialic acid-containing glycolipid s or sialic acid-containing glycoproteins, the ability to decrease the binding of a suppressor of cytokine signaling (SOCS) protein (e.g., SOCS3 protein) to a CD33 protein, the ability to increase the proteasomal degradation of a CD33 protein, the ability to reduce functional expression of CD33 on the surface of circulating dendritic cells, macrophages, monocytes, T cells, and/or microglia, the ability to decrease phosphorylation of Tyr-340 and Tyr-358 by a Src family tyrosine kinase such as LCK and FYN, the ability to decrease recruitment of and binding to the tyrosine-specific protein phosphatases SHP1 and SHP2, the ability to decrease recruitment of and binding to PLC-g1, which acts as a guanine nucleotide, exchange factor for Dynamin-1, the ability to decrease recruitment of and binding to Crk1, the ability to decrease recruitment of and binding to the Spleen tyrosine kinase Syk, the ability to decrease recruitment of and binding to SH3-SH2-SH3 growth factor receptor-bound protein 2 (Grb2), the ability to decrease recruitment of and binding to multiple SH2 containing proteins, the ability to increase intracellular calcium mobilization, the ability to modulate production of pro-inflammatory cytokines IL-1β, IL-8, and TNF-α, the ability to decrease activation of phosphoinositide 3-kinase, the ability to increase the growth of monocytes, macrophages, dendritic cells, T cells, and/or microglia, the ability to increase the survival of monocytes, macrophages, dendritic cells, T cells, and/or microglia, the ability to increase tyrosine phosphorylation on multiple cellular proteins, the ability to increase phagocytic activity of monocytes, macrophages, dendritic cells and/or microglia, the ability to increase cell proliferation of monocytes, macrophages, dendritic cells, T cells, and/or microglia, the ability to increase phosphorylation of signaling molecules that mediates ITAM signaling, the ability to increase the function of pattern recognition receptors, the ability to increase the function of Toll-like receptors, the ability to increases the function of damage-associated molecular pattern (DAMP) receptors, the ability to modulate expression of C—C chemokine receptor 7 (CCR7), and the ability to increase of clearance of cellular and protein debris.

In some embodiments, anti-CD33 antibodies that may be used in the methods of the disclosure have an Fc region that displays reduced binding to one or more Fcg Receptor. Examples of such Fc regions and modifications are provided herein. In some embodiments, the antibody has an Fc isotype listed below.

(xi) Antibody Fc Isotypes with Reduced Binding to Fc Gamma Receptors

In some embodiments, anti-CD33 antibodies that may be used in the methods of the disclosure have reduced binding to Fc gamma receptors, and have an Fc isotype listed in Table C below.

TABLE C
Exemplary anti-CD33 antibody Fc isotypes
with reduced binding to Fc gamma receptor

| Fc Isotype | Mutation (EU numbering scheme) |
|---|---|
| IgG1 | N297A or N297Q and/or D270A |
| IgG1 | D265A, D270A, and/or N297A |
| IgG1 | L234A and L235A |
| IgG2 | V234A and G237A |
| IgG4 | F235A and G237A and E318A |
| | E233P and/or F234V |
| | N297Aor N297Q |
| IgG4 | S228P and L236E |
| | S241P |
| | S241P and L248E |
| | S228P and F234A and L235A |
| IgG2 | H268Q and V309L and A330S and P331S |
| IgG1 | C220S and C226S and C229S and P238S |
| IgG1 | C226S and C229S and E233P and L234V, and L235A |
| IgG1 | E233P and L234V and L235A and G236-deleted |
| | P238A |
| | D265A |
| | N297A |
| | A327Q or A327G |
| | P329A |
| IgG1 | K322A and L234A and L235A |
| IgG1 | L234Fand L235E and P331S |
| IgG1 or IgG4 | T394D |
| IgG2 | C232S or C233S |
| | N297Aor N297Q |
| IgG2 | V234A and G237A and P238S and H268A and V309L and |
| | A330S and P331S |
| IgG1, IgG2, or IgG4 | delta a, b, c, ab, ac, g modifications |
| IgG1 | Any of the above listed mutations together with A330L or L234F |
| | and/or L235E and/or P331S |
| IgG1, IgG2, or IgG4 | Any of the above listed mutations together with M252Y and/or |
| | S254T and/or T256E |

In certain embodiments, anti-CD33 antibodies that may be used in the methods of the disclosure have an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the Fc region contains one or more modifications. For example, in some embodiments, the Fc region contains one or more amino acid substitutions (e.g., relative to a wild-type Pc region of the same isotype).

In some embodiments, the one or more amino acid substitutions are selected from N297A, N297Q (Bolt S et al. (1993) Eur J Immunol 23:403-411), D265A, D270A, L234A, L235A (McEarchern et al., (2007) Blood, 109:1185-1192), C226S, C229S (McEarchern et al., (2007) Blood, 109:1185-1192), P238S (Davis et al., (2007) J Rheumatol, 34:2204-2210), E233P, L234V (McEarchern et al., (2007) Blood, 109:1185-1192), P238A, A327Q, A327G, P329A (Shields R L, et al., (2001) J Biol Chem. 276 (9): 6591-604), K322A, L234F, L235E (Hezareh, et al., (2001) J Virol 75, 12161-12168; Oganesyan et al., (2008). Acta Crystallographica 64, 700-704), P331S (Oganesyan et al., (2008) Acta Crystallographica 64, 700-704), T394D (Wilkinson et al. (2013) MAbs 5 (3): 406-417), A330L, M252Y, S254T, and/or T256E, where the amino acid position is according to the EU or Kabat numbering convention. In certain embodiments, the Fc region further includes an amino acid deletion at a position corresponding to glycine 236 according to the EU or Kabat numbering convention.

In some embodiments, anti-CD33 antibodies that may be used in the methods of the disclosure have an IgG1 isotype with a heavy chain constant region that contains a C220S amino acid substitution according to the EU or Kabat numbering convention. In some embodiments, the Fc region further contains one or more additional amino acid substitutions selected from A330L, L234F; L235E, and/or P331S according to EU or Kabat numbering convention. In certain embodiments, the anti-CD33 antibody has an IgG2 isotype. In some embodiments, the anti-CD33 antibody contains a human IgG2 constant region. In some embodiments, the human IgG2 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from P238S, V234A, G237A, H268A, H268Q, H268E, V309L, N297A, N297Q, V309L, A330S, P331S, C232S, C233S, M252Y, S254T, and/or T256E, where the amino acid position is according to the EU or Kabat numbering convention (Vafa O. et al., (2014) Methods 65:114-126).

In certain embodiments, anti-CD33 antibodies that may be used in the methods of the disclosure have an IgG4 isotype. In some embodiments, the anti-CD33 antibody contains a human IgG4 constant region. In some embodiments, the human IgG4 constant region includes an Fc region. In some embodiments, the Fc region contains one or more modifications. For example, in some embodiments, the Fc region contains one or more amino acid substitutions (e.g., relative to a wild-type Fc region of the same isotype). In some embodiments, the one or more amino acid substitutions are selected from E233P, F234V, L235A, G237A, E318A (Hutchins et al. (1995) Proc Natl Acad Sci USA, 92:11980-11984), S228P, L234A/F234A, L236E, S241P, L248E (Reddy et al., (2000) J Immunol, 164:1925-1933; Angal et al., (1993) Mol Immunol. 30 (1): 105-8; U.S. Pat. No. 8,614,299 B2; Vafa O. et al., (2014) Methods 65:114-126), T394D, M252Y, S254T, T256E, N297A, and/or N297Q, where the amino acid position is according to the EU or Kabat numbering convention. In some embodiments the antibody has an IgG4 isotype, and comprises an S228P amino acid substitution at residue position 228, an F234A amino acid substitution at residue position 234, and an L235A amino acid substitution at residue position 235 (residue position according to EU numbering).

In some embodiments, the Fc region further contains one or more additional amino acid substitutions selected from a M252Y, S254T, and/or T256E, where the amino acid position is according to the EU or Kabat numbering convention.

(xii) Further IgG Mutations

In some embodiments, one or more of the IgG1 variants described herein may be combined with an A330L mutation (Lazar et al., (2006) Proc Natl Acad Sci USA, 103:4005-4010), or one or more of L234F, L235E, and/or P331S mutations (Sazinsky et al., (2008) Proc Natl Acad Sci USA, 105:20167-20172), where the amino acid position is according to the EU or Kabat numbering convention, to eliminate complement activation. In some embodiments, the IgG variants described herein may be combined with one or more mutations to enhance the anti-CD33 antibody half-life in human serum (e.g. M252Y, S254T, T256E mutations according to the EU or Kabat numbering convention) (Dall'Acqua et al., (2006) J Biol Chem, 281:23514-23524; and Strohl e al., (2009) Current Opinion in Biotechnology, 20:685-691).

In some embodiments, an IgG4 variant of the present disclosure may be combined with an S228P mutation according to the EU or Kabat numbering convention (Angal et al., (1993) Mol Immunol, 30:105-108) and/or with one or more mutations described in Peters et al., (2012) J Biol Chem. 13; 287 (29): 24525-33) to enhance antibody stabilization.

(xiii) Pharmacokinetics of Anti-CD33 Antibodies

In some embodiments, the half-life, e.g., the terminal half-life, of anti-CD33 antibodies for use in the methods of the present disclosure in plasma or serum is at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days. In some embodiments, the half-life, e.g., the terminal half-life, of anti-CD33 antibodies for use in the methods of the present disclosure in plasma or serum is about 3 days, about 4 days, about 5 days, about 6 days, about 7 days, about 8 days, about 9 days, about 10 days, about 11 days, about 12 days, about 13 days, about 14 days, or about 15 days. In some embodiments, the half-life, e.g., the terminal half-life, of anti-CD33 antibodies for use in the methods of the present disclosure in plasma or serum is between about 3 days and about 12 days, or between about 3 days and 15 days (e.g., any of about 3 days, about 4 days, about 5 days, about 6 days, about 7 days, about 8 days, about 9 days, about 10 days, about 11 days, about 12 days, about 13 days, about 14 days, or about 15 days). In some embodiments, the terminal half-life of the anti-CD33 antibody is about 4 days. In some embodiments, the terminal half-life of the anti-CD33 antibody is about 10 days. In some embodiments, the terminal half-life of the anti-CD33 antibody is about 11 days. In some embodiments, the half-life, e.g., the terminal half-life, of the anti-CD33 antibody following administration of a single dose of the antibody at 1.6 mg/kg is about 4 days. In some embodiments, the half-life, e.g., the terminal half-life, of the anti-CD33 antibody following administration of a single dose of the antibody at 1.6 mg/kg is about 5 days. In some embodiments, the half-life, e.g., the terminal half-life, of the anti-CD33 antibody following administration of a single dose of the antibody at 5 mg/kg is about 7 days. In some embodiments, the half-life, e.g., the terminal half-life, of the anti-CD33 antibody following administration of a single dose of the antibody at 5 mg/kg is about 8 days. In some embodiments, the half-life, e.g., the terminal half-life, of the anti-CD33 antibody following administration of a single dose of the antibody at 15 mg/kg is about 10 days. In some embodiments, the half-life, e.g., the terminal half-life, of the anti-CD33 antibody following administration of a single dose of the antibody at 15 mg/kg is about 11 days. In some embodiments, the half-life, e.g., the terminal half-life, of the anti-CD33 antibody following administration of a single dose of the antibody at 30 mg/kg is about 8 or 9 days. In some embodiments, the half-life, e.g., the terminal half-life, of the anti-CD33 antibody following administration of a single dose of the antibody at 30 mg/kg is about 10 days. In some embodiments, the half-life, e.g., the terminal half-life, of the anti-CD33 antibody following administration of a single dose of the antibody at 60 mg/kg is about 6, 7, or 8 days.

In some embodiments, the half-life, e.g., the terminal half-life, of an anti-CD33 antibody may be determined using any method known in the art. In some embodiments, the half-life, e.g., the terminal half-life, of an anti-CD33 antibody is determined using enzyme-linked immunosorbent assay (ELISA). In some embodiments, the half-life, e.g., the terminal half-life, of an anti-CD33 antibody is determined in blood of the individual. In some embodiments, the half-life, e.g., the terminal half-life, of an anti-CD33 antibody is determined in plasma of the individual. In some embodiments, the half-life, e.g., the terminal half-life, of an anti-CD33 antibody is determined in serum of the individual. In some embodiments, the half-life, e.g., the terminal half-life, of an anti-CD33 antibody is determined in CSF of the individual.

(xiv) Nucleic Acids, Vectors, and Host cells

Anti-CD33 antibodies for use in the methods of present disclosure may be produced using recombinant methods and compositions, e.g., as described in U.S. Pat. No. 4,816,567. In some embodiments, isolated nucleic acids having a nucleotide sequence encoding any of the anti-CD33 antibodies of the present disclosure are provided. Such nucleic acids may encode an amino acid sequence containing the VL and/or an amino acid sequence containing the VH of the anti-CD33 antibody (e.g., the light and/or heavy chains of the antibody). In some embodiments, one or more vectors (e.g., expression vectors) containing such nucleic acids are provided. In some embodiments, a host cell containing such nucleic acid is also provided. In some embodiments, the host cell contains (e.g., has been transduced with): (1) a vector containing a nucleic acid that encodes an amino acid sequence containing the VL of the antibody and an amino acid sequence containing the VH of the antibody, or (2) a first vector containing a nucleic acid that encodes an amino acid sequence containing the VL of the antibody and a second vector containing a nucleic acid that encodes an amino acid sequence containing the VH of the antibody. In some embodiments, the host cell is eukaryotic, e.g., a Chinese Hamster Ovary (CHO) cell or lymphoid cell (e.g., Y0, NS0, Sp20 cell).

Methods of making an anti-CD33 antibody of the present disclosure are provided. In some embodiments, the method includes culturing a host cell of the present disclosure containing a nucleic acid encoding the anti-CD33 antibody, under conditions suitable for expression of the antibody. In some embodiments, the antibody is subsequently recovered from the host cell (or host cell culture medium).

For recombinant production of an anti-CD33 antibody of the present disclosure, a nucleic acid encoding the anti-CD33 antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the anti-CD33 antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a nucleic acid of the present disclosure. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-CD33 antibody of the present disclosure.

Suitable host cells for cloning or expression of antibody-encoding vectors include prokaryotic or eukaryotic cells. For example, anti-CD33 antibodies of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, N J, 2003), pp. 245-254, describing expression of antibody fragments in E. coli.). After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross, Nat. Biotech. 22:1409-1414 (2004); and Li et al., Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants.).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### (xv) CD33 Proteins

Anti-CD33 antibodies that may be used in the methods of the disclosure, such as anti-CD33 antibodies known in the art or described herein, are antibodies that interact with or otherwise bind to a region, such as an epitope, within a CD33 protein, such as a mammalian or human CD33 protein. In some embodiments, the antibodies interact with or otherwise bind to a region, such as an epitope, within a CD33 protein with improved/enhanced kinetics (e.g., relative to an anti-CD33 antibody having a heavy chain variable region comprising the sequence of SEQ ID NO: 23 and a light chain variable region comprising the sequence of SEQ ID NO: 24). In some embodiments, the antibodies interact with or otherwise bind to a region, such as an epitope, within a CD33 protein on human cells, such as dendritic cells, with a half-maximal effective concentration (EC50) that is lower than that of a control antibody (e.g., relative to an anti-CD33 antibody having a heavy chain variable region comprising the sequence of SEQ ID NO: 23 and a light chain variable region comprising the sequence of SEQ ID NO: 24). In some embodiments, anti-CD33 antibodies of the present disclosure bind to a CD33 protein and modulate one or more CD33 activities after binding to the CD33 protein, for example, an activity associated with CD33 expression on a cell. CD33 proteins of the present disclosure include, without limitation, a mammalian CD33 protein, human CD33 protein, mouse CD33 protein, a cynomolgus monkey CD33 protein, and rat CD33 protein.

CD33 is variously referred to as a CD33 molecule, Siglec3, Siglec-3, CD33 antigen (Gp67), P67, Gp67, sialic acid-binding-Ig-like lectin 3, myeloid cell surface antigen CD33, or FLJ00391.

CD33 is an immunoglobulin-like receptor primarily expressed on myeloid lineage cells, including without limitation, macrophages, dendritic cells, osteoclasts, monocytes, and microglia. In some embodiments, CD33 forms a receptor-signaling complex with CD64. In some embodiments, CD33 signaling results in the downstream inhibition of PI3K or other intracellular signals. On myeloid cells, Toll-like receptor (TLR) signals are important for the inhibition of CD33 activities, e.g., in the context of an infection response. TLRs also play a key role in the pathological inflammatory response, e.g., TLRs expressed in macrophages and dendritic cells.

The amino acid sequence of human CD33 is set forth below as SEQ ID NO: 25:

MPLLLLLPLLWAGALAMDPNFWLQVQESVTVQEGLCVLVPCTFFHPIPYY
DKNSPVHGYWFREGAIISRDSPVATNKLDQEVQEETQGRFRLLGDPSRNN
CSLSIVDARRRDNGSYFFRMERGSTKYSYKSPQLSVHVTDLTHRPKILIP
GTLEPGHSKNLTCSVSWACEQGTPPIFSWLSAAPTSLGPRITHSSVLIIT
PRPQDHGTNLTCQVKFAGAGVTTERTIQLNVTYVPQNPTTGIFPGDGSGK
QETRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNP
SKDTSTEYSEVRTQ

In some embodiments, the CD33 protein is a pre-protein that includes a signal sequence. In some embodiments, the CD33 protein is a mature protein. In some embodiments, the mature CD33 protein does not include a signal sequence. In some embodiments, the mature CD33 protein is expressed on a cell. In some embodiments, the mature CD33 protein is expressed on a cell, such as the surface of a cell, including, without limitation, human dendritic cells, human macrophages, human monocytes, human osteoclasts, human neutrophils, human T cells, human T helper cell, human cytotoxic T cells, human granulocytes, and human microglia. Anti-CD33 antibodies that may be used in the methods of the disclosure, such as anti-CD33 antibodies known in the art or described herein, may bind any of the CD33 proteins of the present disclosure expressed on any cell disclosed herein.

CD33 proteins of the present disclosure, such as human CD33, contain several domains, including without limitation, a signal sequence located at amino acid residues 1-17 of SEQ ID NO: 25, an extracellular immunoglobulin-like variable-type (IgV) domain located at amino acid residues 19-135 of SEQ ID NO: 25, an Ig-like C2-type domain located at amino acid residues 145-228 of SEQ ID NO: 25, a transmembrane domain located at amino acid residues 260-282 of SEQ ID NO: 25, an ITIM motif 1 located at amino acid residues 338-343 of SEQ ID NO: 25, and an ITIM motif 2 located at amino acid residues 356-361 of SEQ ID NO: 25. As one of skill in the art will appreciate, the beginning and ending residues of the domains of the present disclosure may vary depending upon the computer modeling program used or the method used for determining the domain.

Anti-CD33 antibodies that may be used in the methods of the disclosure, such as anti-CD33 antibodies known in the art or described herein, may bind to a human CD33 protein, or a homolog thereof, including without limitation a mammalian CD33 protein and CD33 orthologs from other species. In some embodiments, anti-CD33 antibodies for use in the methods of the disclosure, such as anti-CD33 antibodies known in the art or described herein, bind to a human CD33, or homolog thereof, with improved/enhanced binding kinetics (e.g., relative to an anti-CD33 antibody having a heavy chain variable region comprising the sequence of SEQ ID NO: 23 and a light chain variable region comprising the sequence of SEQ ID NO: 24).

Accordingly, as used herein a “CD33” protein of the present disclosure includes, without limitation, a mammalian CD33 protein, human CD33 protein, primate CD33 protein, mouse CD33 protein, and rat CD33 protein. Additionally, anti-CD33 antibodies for use in the methods of the disclosure, such as anti-CD33 antibodies known in the art or described herein, may bind an epitope within a human CD33 protein, or a primate CD33 protein. In some embodiments, anti-CD33 antibodies for use in the methods of the disclosure, such as anti-CD33 antibodies known in the art or described herein, may bind specifically to a human CD33 protein.

In some embodiments, anti-CD33 antibodies for use in the methods of the disclosure, such as anti-CD33 antibodies known in the art or described herein, may bind CD33 in a pH dependent manner. In some embodiments, anti-CD33 antibodies for use in the methods of the disclosure, such as anti-CD33 antibodies known in the art or described herein, can bind to CD33 at a neutral pH and be internalized without dissociating from the CD33 protein. Alternatively, at an acidic pH, anti-CD33 antibodies for use in the methods of the disclosure, such as anti-CD33 antibodies known in the art or described herein, may dissociate from CD33 once they are internalized and are then degraded by endosome/lysosome pathway. In certain embodiments, anti-CD33 antibodies for use in the methods of the disclosure, such as anti-CD33 antibodies known in the art or described herein, bind CD33 at a pH that ranges from 5.5 to 8.0, from 5.5 to 7.5, from 5.5 to 7.0, from 5.5 to 6.5, from 5.5 to 6.0, from 6.0 to 8.0, from 6.5 to 8.0, from 7.0 to 8.0, from 7.5 to 8.0, from 6.0 to 7.5, from 6.0 to 7.0, or from 6.5 to 7.5. In certain embodiments, anti-CD33 antibodies for use in the methods of the disclosure, such as anti-CD33 antibodies known in the art or described herein, dissociate from CD33 at a pH of less than 6.0, less than 5.5, less than 5.0, less than 4.5, less than 4.0, less than 3.5, less than 3.0, less than 2.5, or less than 2.0.

In some embodiments, anti-CD33 antibodies for use in the methods of the disclosure, such as anti-CD33 antibodies known in the art or described herein, bind to a wild-type CD33 protein of the present disclosure, naturally occurring variants thereof, and/or disease variants thereof.

In some embodiments, anti-CD33 antibodies for use in the methods of the disclosure, such as anti-CD33 antibodies known in the art or described herein, bind a variant of human CD33, wherein the variant contains a single nucleotide polymorphism (SNP) rs3865444C with a (C) nucleotide. In some embodiments, anti-CD33 antibodies for use in the methods of the disclosure, such as anti-CD33 antibodies known in the art or described herein, that decrease cellular levels of CD33 and/or that bind or interact with CD33, bind to a variant of human CD33, wherein the variant contains a SNP rs3865444 with an (A) nucleotide. In some embodiments, anti-CD33 antibodies for use in the methods of the disclosure, such as anti-CD33 antibodies known in the art or described herein, bind a variant of human CD33, wherein the variant contains a SNP rs3865444^{AC} or rs3865444^{CC}.

In some embodiments, anti-CD33 antibodies for use in the methods of the disclosure, such as anti-CD33 antibodies known in the art or described herein, that decrease cellular levels of CD33 and/or that bind or interact with CD33, bind a variant of human CD33, wherein the variant contains a SNP rs35112940 with GG nucleotides, AA nucleotides, or AG nucleotides. In some embodiments, anti-CD33 antibodies for use in the methods of the disclosure, such as anti-CD33 antibodies known in the art or described herein, that decrease cellular levels of CD33 and/or that bind or interact with CD33, bind a variant of human CD33, wherein the variant contains a SNP rs12459419 with CC, CT or TT genotypes. In certain embodiments, an individual according to any of the methods of the disclosure is homozygous or heterozygous for the coding SNPs rs1803 with GG nucleotides, CG nucleotides, or CC nucleotides.

In some embodiments, anti-CD33 antibodies for use in the methods of the disclosure, such as anti-CD33 antibodies known in the art or described herein, that decrease cellular levels of CD33 and/or that bind or interact with CD33, bind to a CD33 protein expressed on the surface of a cell including, without limitation, human dendritic cells, human macrophages, human monocytes, human osteoclasts, human neutrophils, human T cells, human T helper cell, human cytotoxic T cells, human granulocytes, and human microglia. In some embodiments, anti-CD33 antibodies for use in the methods of the disclosure, such as anti-CD33 antibodies known in the art or described herein, that decrease cellular levels of CD33 and/or that bind or interact with CD33, bind to a CD33 protein expressed on the surface of a cell and modulate (e.g., induce or inhibit) at least one CD33 activity of the present disclosure after binding to the surface-expressed CD33 protein. In some embodiments of the present disclosure, anti-CD33 antibodies for use in the methods of the disclosure, such as anti-CD33 antibodies known in the art or described herein, bind specifically to a CD33 protein. In some embodiments of the present disclosure, anti-CD33 antibodies for use in the methods of the disclosure, such as anti-CD33 antibodies known in the art or described herein, further bind to at least one additional Siglec protein. In some embodiments, anti-CD33 antibodies for use in the methods of the disclosure, such as anti-CD33 antibodies known in the art or described herein, modulate one or more activities of the at least one additional Siglec protein or of a cell expressing the at least one additional Siglec protein.

(xvi) CD33 Ligands

CD33 proteins of the present disclosure can interact with (e.g., bind to) one or more CD33 ligands.

Exemplary CD33 ligands include, without limitation, sialic acid, sialic acid-containing glycolipids, sialic acid-containing glycoproteins, alpha-2,6-linked sialic acid-containing glycolipids, alpha-2,6-linked sialic acid-containing glycoproteins, alpha-2,3-linked sialic acid-containing glycolipids, alpha-2,3-linked sialic acid-containing glycoproteins, alpha-1-acid glycoprotein (AGP), CD24 protein, gangliosides (e.g., glycolipids containing a ceramide linked to a sialylated glycan), secreted mucins, CD33 ligands expressed on red blood cells, CD33 ligands expressed on bacterial cells, CD33 ligands expressed on apoptotic cells, CD33 ligands expressed on tumor cells, CD33 ligands expressed on viruses, CD33 ligands expressed on dendritic cells, CD33 ligands expressed on nerve cells, CD33 ligands expressed on glial cells, CD33 ligands expressed on microglia, CD33 ligands expressed on astrocytes, CD33 ligands on beta amyloid plaques, CD33 ligands on Tau tangles, CD33 ligands on disease-causing proteins, CD33 ligands on disease-causing peptides, CD33 ligands expressed on macrophages, CD33 ligands expressed on natural killer cells, CD33 ligands expressed on T cells, CD33 ligands expressed on T helper cells, CD33 ligands expressed on cytotoxic T cells, CD33 ligands expressed on B cells, CD33 ligands expressed on tumor-imbedded immunosuppressor dendritic cells, CD33 ligands expressed on tumor-imbedded immunosuppressor macrophages, CD33 ligands expressed on myeloid-derived suppressor cells, and CD33 ligands expressed on regulatory T cells. In some embodiments, CD33 ligands of the present disclosure are gangliosides. Gangliosides generally share a common lacto-ceramide core and one or more sialic acid residues.

Further examples of suitable ganglioside ligands are listed in Table D. Generally, a ganglioside is a molecule composed of a glycosphingolipid with one or more sialic acids (e.g., n-acetyl-neuraminic acid, NANA) linked on the sugar chain.

TABLE D
Structures of exemplary ganglioside CD33 ligands

GM2-1 = aNeu5Ac(2-3)bDGalp(1-?)bDGalNAc(1-?)bDGalNAc(1-?)bDGlcp(1-1)Cer
GM3 = aNeu5Ac(2-3)bDGalp(1-4)bDGlcp(1-1)Cer
GM2, GM2a(?) = bDGalpNAc(1-4)[aNeu5Ac(2-3)]bDGalp(1-4)bDGlcp(1-1)Cer
GM2b(?) = aNeu5Ac(2-8)aNeu5Ac(2-3)bDGalp(1-4)bDGlcp(1-1)Cer
GM1, GM1a = bDGalp(1-3)bDGalNAc[aNeu5Ac(2-3)]bDGalp(1-4)bDGlcp(1-1)Cer
asialo-GM1, GA1 = bDGalp(1-3)bDGalpNAc(1-4)bDGalp(1-4)bDGlcp(1-1)Cer
asialo-GM2, GA2 = bDGalpNAc(1-4)bDGalp(1-4)bDGlcp(1-1)Cer
GM1b = aNeu5Ac(2-3)bDGalp(1-3)bDGalNAc(1-4)bDGalp(1-4)bDGlcp(1-1)Cer
GD3 = aNeu5Ac(2-8)aNeu5Ac(2-3)bDGalp(1-4)bDGlcp(1-1)Cer
GD2 = bDGalpNAc(1-4)[aNeu5Ac(2-8)aNeu5Ac(2-3)]bDGalp(1-4)bDGlcp(1-1)Cer
GD1a = aNeu5Ac(2-3)bDGalp(1-3)bDGalNAc(1-4)[aNeu5Ac(2-3)]bDGalp(1-4)bDGlcp(1-1)Cer
GD1alpha = aNeu5Ac(2-3)bDGalp(1-3)bDGalNAc(1-4)[aNeu5Ac(2-6)]bDGalp(1-4)bDGlcp(1-1)Cer
GD1b = bDGalp(1-3)bDGalNAc(1-4)[aNeu5Ac(2-8)aNeu5Ac(2-3)]bDGalp(1-4)bDGlcp(1-1)Cer
GT1a = aNeu5Ac(2-8)aNeu5Ac(2-3)bDGalp(1-3)bDGalNAc(1-4)[aNeu5Ac(2-3)]bDGalp(1-4)bDGlcp(1-1)Cer
GT1, GT1b = aNeu5Ac(2-3)bDGalp(1-3)bDGalNAc(1-4)[aNeu5Ac(2-8)aNeu5Ac(2-3)]bDGalp(1-4)bDGlcp(1-1)Cer
OAc-GT1b = aNeu5Ac(2-3)bDGalp(1-3)bDGalNAc(1-4)aXNeu5Ac9Ac(2-8)aNeu5Ac(2-3)]bDGalp(1-4)bDGlcp(1-1)Cer
GT1c = bDGalp(1-3)bDGalNAc(1-4)[aNeu5Ac(2-8)aNeu5Ac(2-8)aNeu5Ac(2-3)]bDGalp(1-4)bDGlcp(1-1)Cer
GT3 = aNeu5Ac(2-8)aNeu5Ac(2-8)aNeu5Ac(2-3)bDGal(1-4)bDGlc(1-1)CerGQ1b = aNeu5Ac(2-
8)aNeu5Ac(2-3)bDGalp(1-3)bDGalNAc(1-4)[aNeu5Ac(2-8)aNeu5Ac(2-3)]bDGalp(1-4)bDGlcp(1-1)Cer
GGal = aNeu5Ac(2-3)bDGalp(1-1)Cer
where:
aNeu5Ac = 5-acetyl-alpha-neuraminic acid
aNeu5Ac9Ac = 5,9-diacetyl-alpha-neuraminic acid
bDGalp = beta-D-galactopyranose
bDGalpNAc = N-acetyl-beta-D-galactopyranose
bDGlcp = beta-D-glucopyranose
Cer = ceramide (general N-acylated sphingoid)

(xvii) Pharmaceutical Compositions

Anti-CD33 antibodies, such as anti-CD33 antibodies known in the art and/or described herein, can be incorporated into a variety of formulations for therapeutic administration by combining the anti-CD33 antibodies with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

A pharmaceutical composition of the present disclosure can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include, without limitation, sulfate, gluconate, citrate and phosphate. The polypeptides of a composition can also be complexed with molecules that enhance their in vivo attributes. Such molecules include, without limitation, carbohydrates, polyamines, amino acids, other peptides, ions (e.g., sodium, potassium, calcium, magnesium, manganese), and lipids.

Further examples of formulations that are suitable for various types of administration can be found in Remington's Pharmaceutical Sciences, Mace Publishing Company, Philadelphia, PA, 17th ed. (1985). For a brief review of methods for drug delivery, see, Langer, Science 249:1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc., in order to achieve an increase in molecular weight.

Other strategies for increasing retention include the entrapment of an anti-CD33 antibody of the present disclosure in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like, and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, and half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers may be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the present disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### F. Exemplary Treatment Regimens

Anti-CD33 antibodies, such anti-CD33 antibodies known in the art and/or described herein, may be administered to an individual in need of treatment with the antibody in accordance with known methods, such as intravenous administration as a bolus or by continuous infusion over a period of time, by intramuscular, intraperitoneal, intracerobrospinal, intracranial, intraspinal, subcutaneous, intra-articular, intrasynovial, intrathecal, oral, topical, or inhalation routes.

Dosages and desired drug concentration of an anti-CD33 antibody may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of an anti-CD33 antibody, normal dosage amounts may vary from about 10 ng/kg up to about 100 mg/kg of an individual's body weight, preferably about 1.6 mg/kg to about 60 mg/kg, or about 1.6 mg/kg to about 15 mg/kg, depending upon the route of administration. For repeated administrations over several days or longer, depending on the severity of the disease or injury to be treated, the treatment is sustained until a desired suppression of symptoms is achieved.

In some embodiments, the anti-CD33 antibody is administered at a dose of between about 1.6 mg/kg and about 15 mg/kg about once every twelve weeks or more frequently. In some embodiments, the anti-CD33 antibody is administered at a dose of between about 1.6 mg/kg and about 15 mg/kg about once every two weeks to about once every twelve weeks. In some embodiments, the anti-CD33 antibody is administered at a dose of about 1.6 mg/kg, about 5 mg/kg, about 7.5 mg/kg, about 10 mg/kg, or about 15 mg/kg. In some embodiments, the anti-CD33 antibody is administered once every two weeks, once every three weeks, once every four weeks, once every five weeks, once every six weeks, once every seven weeks, once every eight weeks, once every nine weeks, once every ten weeks, once every eleven weeks, or once every twelve weeks. In some embodiments, the anti-CD33 antibody is administered once every two weeks at a dose of about 1.6 mg/kg. In some embodiments, the anti-CD33 antibody is administered once every four weeks at a dose of about 1.6 mg/kg. In some embodiments, the anti-CD33 antibody is administered once every four weeks at a dose of about 15 mg/kg. In some embodiments, the anti-CD33 antibody is administered once every five weeks at a dose of about 15 mg/kg. In some embodiments, the anti-CD33 antibody is administered once every six weeks at a dose of about 15 mg/kg. In some embodiments, the anti-CD33 antibody is administered once every seven weeks at a dose of about 15 mg/kg. In some embodiments, the anti-CD33 antibody is administered once every eight weeks at a dose of about 15 mg/kg.

In some embodiments, the anti-CD33 antibody is administered at a dose of between about 1.6 mg/kg and about 60 mg/kg (e.g., any of about 1.6 mg/kg, about 5 mg/kg, about 7.5 mg/kg, about 10 mg/kg, about 15 mg/kg, about 30 mg/kg, about 35 mg/kg, about 40 mg/kg, about 45 mg/kg, about 50 mg/kg, about 55 mg/kg, or about 60 mg/kg) on day 1 of a treatment period, and once every twelve weeks or more frequently (e.g., any of once every two weeks, once every three weeks, once every four weeks, once every five weeks, once every six weeks, once every seven weeks, once every eight weeks, once every nine weeks, once every ten weeks, once every eleven weeks, or once every 12 weeks) thereafter. In some embodiments, the anti-CD33 antibody is administered at a dose of about 1.6 mg/kg on day 1 of a treatment period and once every two weeks thereafter. In some embodiments, the anti-CD33 antibody is administered at a dose of about 1.6 mg/kg on day 1 of a treatment period and once every four weeks thereafter. In some embodiments, the anti-CD33 antibody is administered at a dose of about 15 mg/kg on day 1 of a treatment period and once every four weeks thereafter. In some embodiments, the anti-CD33 antibody is administered at a dose of about 15 mg/kg on day 1 of a treatment period and once every five weeks thereafter. In some embodiments, the anti-CD33 antibody is administered at a dose of about 15 mg/kg on day 1 of a treatment period and once every six weeks thereafter. In some embodiments, the anti-CD33 antibody is administered at a dose of about 15 mg/kg on day 1 of a treatment period and once every seven weeks thereafter. In some embodiments, the anti-CD33 antibody is administered at a dose of about 15 mg/kg on day 1 of a treatment period and once every eight weeks thereafter. In some embodiments, the anti-CD33 antibody is administered at a dose of about 15 mg/kg on day 1 of a treatment period and once every nine weeks thereafter. In some embodiments, the anti-CD33 antibody is administered at a dose of about 15 mg/kg on day 1 of a treatment period and once every ten weeks thereafter. In some embodiments, the anti-CD33 antibody is administered at a dose of about 15 mg/kg on day 1 of a treatment period and once every eleven weeks thereafter. In some embodiments, the anti-CD33 antibody is administered at a dose of about 15 mg/kg on day 1 of a treatment period and once every twelve weeks thereafter.

Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 g/kg, about 1 mg/kg, and about 2/mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. In some embodiments, progress of the therapy is monitored based on sCD33 levels as described in the present disclosure. The dosing regimen, including the anti-CD33 antibody administered, can vary over time independently of the dose used.

Dosages for a particular anti-CD33 antibody may be determined empirically in individuals who have been given one or more administrations of the anti-CD33 antibody. Individuals are given incremental doses of an anti-CD33 antibody. To assess efficacy of an anti-CD33 antibody, a clinical symptom of any of the diseases or injuries of the present disclosure (e.g., dementia, frontotemporal dementia, Alzheimer's disease, vascular dementia, mixed dementia, and taupathy disease) can be monitored. To assess efficacy of an anti-CD33 antibody, sCD33 levels may be assessed as described in the present disclosure.

Administration of an anti-CD33 antibody of the present disclosure can be continuous or intermittent, depending, for example, on the recipient's physiological condition, whether the purpose of the administration is therapeutic or prophylactic, and other factors known to skilled practitioners. The administration of an anti-CD33 antibody may be essentially continuous over a preselected period of time or may be in a series of spaced doses.

Guidance regarding particular dosages and methods of delivery is provided in the literature. See, for example, U.S. Pat. Nos. 4,657,760; 5,206,344; or 5,225,212. It is within the scope of the present disclosure that different formulations will be effective for different treatments and different diseases or injuries, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the disease or injury, the treatment is sustained until a desired suppression of symptoms occurs. However, other dosage regimens may be useful.

In some embodiments, progress of any of the treatment regimens described herein is monitored based on sCD33 levels as described in the present disclosure.

TABLE E
of the Disclosure.

| | | |
|---|---|---|
| Description | Sequence | SEQ ID NO |

Exemplary Anti-CD33 Antibody AB64.1.2 Amino Acid Sequences

| | | |
|---|---|---|
| AB64.1.2 | GYTFTDYNLH | 1 |
| HVR-H1 | | |
| AB64.1.2 | FIYPSNRITG | 2 |
| HVR-H2 | | |
| AB64.1.2 | SDVDYFDY | 3 |
| HVR-H3 | | |
| AB64.1.2 | RASQSVSTSTYSYMH | 4 |
| HVR-L1 | | |
| AB64.1.2 | YASNLES | 5 |
| HVR-L2 | | |
| AB64.1.2 | QHSWEIPLT | 6 |
| HVR-L3 | | |
| AB64.1.2 | QVQLVQSGAEVKKPGASVKISCKASGYTFTDYNLHWVRQAPG | 7 |
| heavy chain | QGLEWIGFIYPSNRITGYAQKFQGRATLTVDNSTSTAYMELS | |
| variable | SLRSEDTAVYYCARSDVDYFDYWGQGTLLTVSS | |
| region | | |
| AB64.1.2 | DIVLTQSPDSLAVSLGERATINCRASQSVSTSTYSYMHWYQQ | 8 |
| light chain | KPGQPPKLLIKYASNLESGVPDRFSGSGSGTDFTLTISSLQA | |
| variable | EDVAVYYCQHSWEIPLTFGQGTKLEIK | |
| region | | |
| AB64.1.2 | QVQLVQSGAEVKKPGASVKISCKASGYTFTDYNLHWVRQAPG | 9 |
| heavy chain | QGLEWIGFIYPSNRITGYAQKFQGRATLTVDNSTSTAYMELS | |
| | SLRSEDTAVYYCARSDVDYFDYWGQGTLLTVSSASTKGPSVF | |
| | PLAPCSRSTSESTAALGCLVKDYFPEPVTVSWNSGALTSGVH | |
| | TFPAVLQSSGLYSLSSVVTVPSSNFGTQTYTCNVDHKPSNTK | |
| | VDKTVERKCCVECPPCPAPPVAGPSVFLFPPKPKDTLMISRT | |
| | PEVTCVVVDVSHEDPEVQFNWYVDGVEVHNAKTKPREEQENS | |
| | TFRVVSVLTVVHQDWLNGKEYKCKVSNKGLPAPIEKTISKTK | |
| | GQPREPQVYTLPPSREEMTKNQVSLTCLVKGFYPSDIAVEWE | |
| | SNGQPENNYKTTPPMLDSDGSFFLYSKLTVDKSRWQQGNVES | |
| | CSVMHEALHNHYTQKSLSLSPGK | |
| AB64.1.2 | QVQLVQSGAEVKKPGASVKISCKASGYTFTDYNLHWVRQAPG | 10 |
| heavy chain | QGLEWIGFIYPSNRITGYAQKFQGRATLTVDNSTSTAYMELS | |
| | SLRSEDTAVYYCARSDVDYFDYWGQGTLLTVSSASTKGPSVF | |
| | PLAPCSRSTSESTAALGCLVKDYFPEPVTVSWNSGALTSGVH | |
| | TFPAVLQSSGLYSLSSVVTVPSSNFGTQTYTCNVDHKPSNTK | |
| | VDKTVERKCCVECPPCPAPPVAGPSVFLFPPKPKDTLMISRT | |
| | PEVTCVVVDVSHEDPEVQFNWYVDGVEVHNAKTKPREEQENS | |
| | TFRVVSVLTVVHQDWLNGKEYKCKVSNKGLPAPIEKTISKTK | |
| | GQPREPQVYTLPPSREEMTKNQVSLTCLVKGFYPSDIAVEWE | |
| | SNGQPENNYKTTPPMLDSDGSFFLYSKLTVDKSRWQQGNVES | |
| | CSVMHEALHNHYTQKSLSLSPG | |
| AB64.1.2 | DIVLTQSPDSLAVSLGERATINCRASQSVSTSTYSYMHWYQQ | 11 |
| Light chain | KPGQPPKLLIKYASNLESGVPDRFSGSGSGTDFTLTISSLQA | |
| | EDVAVYYCQHSWEIPLTFGQGTKLEIKRTVAAPSVFIFPPSD | |
| | EQLKSGTASVVCLLNNFYPREAKVQWKVDNALQSGNSQESVT | |
| | EQDSKDSTYSLSSTLTLSKADYEKHKVYACEVTHQGLSSPVT | |
| | KSFNRGEC | |

Exemplary Anti-CD33 Antibody 6C7H54 Amino Acid Sequences

| | | |
|---|---|---|
| 6C7H54 | NYEMN | 12 |
| HVR-H1 | | |
| 6C7H54 | EIRLKSNNYVTNYAASVKG | 13 |
| HVR-H2 | | |
| 6C7H54 | AGYYVPFAY | 14 |
| HVR-H3 | | |
| 6C7H54 | TLSSQHSTYTIE | 15 |
| HVR-L1 | | |
| 6C7H54 | LKKEGSHSTGD | 16 |
| HVR-L2 | | |
| 6C7H54 | GVGHTIKEQFVYV | 17 |
| HVR-L3 | | |
| 6C7H54 | EVQLVESGGGLVQPGGSLRLSCAGSGFTFSNYEMNWVRQAPG | 18 |
| heavy chain | KGLEWVAEIRLKSNNYVTNYAASVKGRFTISRDDSKNSVYLQ | |
| variable | MNSLKTEDTGVYYCTRAGYYVPFAYWGQGTLVTVSS | |
| region | | |
| 6C7H54 | QLMLTQSPSASASLGASVKLTCTLSSQHSTYTIEWYQQQPGK | 19 |
| light chain | GPRYLMELKKEGSHSTGDGIPDRESGSSSGAERYLTISSLQS | |
| variable | EDEADYYCGVGHTIKEQFVYVFGGGTKLTVL | |
| region | | |
| 6C7H54 | EVQLVESGGGLVQPGGSLRLSCAGSGFTFSNYEMNWVRQAPG | 20 |
| heavy chain | KGLEWVAEIRLKSNNYVTNYAASVKGRFTISRDDSKNSVYLQ | |
| | MNSLKTEDTGVYYCTRAGYYVPFAYWGQGTLVTVSSASTKGP | |
| | SVFPLAPCSRSTSESTAALGCLVKDYFPEPVTVSWNSGALTS | |
| | GVHTFPAVLQSSGLYSLSSVVTVPSSNFGTQTYTCNVDHKPS | |
| | NTKVDKTVERKCCVECPPCPAPPVAGPSVFLFPPKPKDTLMI | |
| | SRTPEVTCVVVDVSHEDPEVQFNWYVDGVEVHNAKTKPREEQ | |
| | FNSTFRVVSVLTVVHQDWLNGKEYKCKVSNKGLPAPIEKTIS | |
| | KTKGQPREPQVYTLPPSREEMTKNQVSLTCLVKGFYPSDIAV | |
| | EWESNGQPENNYKTTPPMLDSDGSFFLYSKLTVDKSRWQQGN | |
| | VFSCSVMHEALHNHYTQKSLSLSPGK | |
| 6C7H54 | EVQLVESGGGLVQPGGSLRLSCAGSGFTFSNYEMNWVRQAPG | 21 |
| heavy chain | KGLEWVAEIRLKSNNYVTNYAASVKGRFTISRDDSKNSVYLQ | |
| | MNSLKTEDTGVYYCTRAGYYVPFAYWGQGTLVTVSSASTKGP | |
| | SVFPLAPCSRSTSESTAALGCLVKDYFPEPVTVSWNSGALTS | |
| | GVHTFPAVLQSSGLYSLSSVVTVPSSNFGTQTYTCNVDHKPS | |
| | NTKVDKTVERKCCVECPPCPAPPVAGPSVFLFPPKPKDTLMI | |
| | SRTPEVTCVVVDVSHEDPEVQFNWYVDGVEVHNAKTKPREEQ | |
| | FNSTFRVVSVLTVVHQDWLNGKEYKCKVSNKGLPAPIEKTIS | |
| | KTKGQPREPQVYTLPPSREEMTKNQVSLTCLVKGFYPSDIAV | |
| | EWESNGQPENNYKTTPPMLDSDGSFFLYSKLTVDKSRWQQGN | |
| | VFSCSVMHEALHNHYTQKSLSLSPG | |
| 6C7H54 | QLMLTQSPSASASLGASVKLTCTLSSQHSTYTIEWYQQQPGK | 22 |
| light chain | GPRYLMELKKEGSHSTGDGIPDRESGSSSGAERYLTISSLQS | |
| | EDEADYYCGVGHTIKEQFVYVFGGGTKLTVLGQPKAAPSVTL | |
| | FPPSSEELQANKATLVCLISDFYPGAVTVAWKADSSPVKAGV | |
| | ETTTPSKQSNNKYAASSYLSLTPEQWKSHRSYSCQVTHEGST | |
| | VEKTVAPTECS | |

Exemplary Anti-CD33 Antibody Amino Acid Sequences

| | | |
|---|---|---|
| Parental | EVQLQQSGPELVKPGASVKISCKASGYTFTDYNLHWVKLSHG | 23 |
| mouse | KSLEWIGFIYPSNGITGYNQKFKNKATLTVDNSSSTAYMELR | |
| antibody | SLTSEDSAVYYCARSTVDYFDYWGQGTTLTVSS | |
| heavy chain | | |
| variable | | |
| region | | |
| Parental | DIVLTQSPASLAVSLGQRATMSCRASQSVSTSTYSYMHWYQQ | 24 |
| mouse | KPGQPPKLLIKYASNLESGVPARFSGSGSGTDFTLNIHPVEE | |
| antibody | EDTATYYCQHSWEIPLTFGAGTKLELK | |
| light chain | | |
| variable | | |
| region | | |

Additional Sequences of the Disclosure

| | | |
|---|---|---|
| Human | MPLLLLLPLLWAGALAMDPNFWLQVQESVTVQEGLCVLVPCT | 25 |
| CD33 | FFHPIPYYDKNSPVHGYWFREGAIISRDSPVATNKLDQEVQE | |
| | ETQGRFRLLGDPSRNNCSLSIVDARRRDNGSYFFRMERGSTK | |
| | YSYKSPQLSVHVTDLTHRPKILIPGTLEPGHSKNLTCSVSWA | |
| | CEQGTPPIFSWLSAAPTSLGPRITHSSVLIITPRPQDHGTNL | |
| | TCQVKFAGAGVTTERTIQLNVTYVPQNPTTGIFPGDGSGKQE | |
| | TRAGVVHGAIGGAGVTALLALCLCLIFFIVKTHRRKAARTAV | |
| | GRNDTHPTTGSASPKHQKKSKLHGPTETSSCSGAAPTVEMDE | |
| | ELHYASLNFHGMNPSKDTSTEYSEVRTQ | |
| IgG2 | ASTKGPSVFPLAPCSRSTSESTAALGCLVKDYFPEPVTVSWN | 26 |
| isotype CH1 | SGALTSGVHTFPAVLQSSGLYSLSSVVTVPSSNFGTQTYTCN | |
| and hinge | VDHKPSNTKVDKTVERKCCVECPPCP | |
| region | | |

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: A Phase 1 Study Evaluating the Safety, Tolerability, Pharmacokinetics, Pharmacodynamics, and Immunogenicity of Single and Multiple Doses of Anti-CD33 Antibody AB-64.1.2 in Healthy Participants and in Participants with Mild to Moderate Alzheimer's Disease

This Example describes a multi-center, randomized, double-blind, placebo-controlled, dose escalation first in human (FIH) study in healthy adults and in patients with mild to moderate Alzheimer's disease. The study is designed to systematically assess the safety (including immunogenicity) and tolerability, pharmacokinetics (PK), and pharmacodynamics (PD) of anti-CD33 antibody AB-64.1.2 when administered as single ascending doses in healthy participants and as multiple doses in patients with mild to moderate Alzheimer's disease.

### Primary Objective

The primary objective of this study is to evaluate safety, tolerability, pharmacokinetics, and pharmacodynamics of anti-CD33 antibody AB-64.1.2 administered in single ascending doses in healthy participants and multiple doses in patients with mild to moderate Alzheimer's disease.

### Study Design

The study is conducted in 2 parts, as described below.

### Part 1: Single Ascending Dose (SAD) Phase

In the single ascending dose (SAD) phase, up to 49 healthy adult participants are sequentially enrolled in 8 predefined cohorts (cohorts A to H). Three initial cohorts are conducted in 1-3 healthy volunteer participants (all receive active drug, i.e., anti-CD33 antibody AB-64.1.2), then each subsequent cohort has 8 healthy volunteer participants per cohort (6 active: 2 placebo). Additional open label cohorts are added to further assess safety, tolerability, and PD effects in the cerebrospinal fluid (CSF) at alternate timepoints, with up to 8 participants per cohort.

Dosing of the SAD cohorts is as shown in Table 1.

TABLE 1
Dosing regimen for single ascending dose (SAD) cohorts A-H.

Number of Participants

| | | Active | |
|---|---|---|---|
| | | (anti-CD33 | |
| SAD | Dose | antibody | |
| Cohort* | (mg/kg) | AB-64.1.2) | Placebo |
| A | 0.05 | 1-3 | 0 |
| B | 0.2 | 1-3 | 0 |
| C | 0.6 | 1-3 | 0 |
| D | 1.6 | 6 | 2 |
| E | 5 | 6 | 2 |
| F | 15 | 6 | 2 |
| G | 30 | 6 | 2 |
| H | 60 | 6 | 2 |

*Additional open label cohorts are added to further assess safety and tolerability, with up to 8 participants per cohort.

The SAD healthy volunteer phase of the study consists of a screening period, study (treatment) period, follow up visits, and a final follow-up/study completion visit. Screening occurs within 4 weeks prior to enrollment and the first administered dose of study drug on Day 1. All SAD participants are followed for 12 weeks after administration of study treatment. Participants in designated cerebrospinal fluid (CSF) cohorts (i.e., SAD cohorts E, F, G and H) undergo lumbar punctures at screening, on Day 8, and on Day 18 (+1 day), or on a day determined by PK and PD data from previous single dose cohorts where applicable.

Additional single dose cohorts are added as open label cohorts of up to 8 participants per cohort at dose levels not exceeding 15 mg/kg. Participants in these cohorts undergo lumbar punctures at screening (at least 4 days prior to study drug infusion), on Day 8, and on Day 18 (+1 day), or on a day determined by preliminary PK and PD data from previous single dose cohorts where applicable.

### Part 2: Multiple Dose (MD) Phase

In the multiple dose (MD) phase, approximately 12 patients with mild to moderate Alzheimer's disease are enrolled and randomized in 1 cohort (Cohort I; 10 active: 2 placebo).

The MD cohort is initiated only after safety and tolerability up to and including the Day 13 visit for the last participant in the SAD phase has been evaluated. Anti-CD33 antibody AB-64.1.2 is administered via intravenous (IV) infusion in two doses given 4 weeks apart (q4w×2). The dose level used for the 2 study drug infusions is 15 mg/kg, which has been deemed tolerable and is expected to not exceed minimum exposures seen in the 30 mg/kg HV cohort (Cohort G).

The MD phase of the study in patients with mild to moderate Alzheimer's disease consists of a screening period, study (treatment) period, follow-up visits and a final follow-up/study completion visit.

Screening occurs within 6 weeks prior to enrolment and the first administered dose of study drug on Day 1.

Patients are administered the Mini-Mental State Examination (MMSE), Repeatable Battery for the Assessment of Neuropsychological Status (RBANS), and Clinical Dementia Rating (CDR), and undergo magnetic resonance imaging (MRI, including but not limited to fluid-attenuated inversion recovery [FLAIR] and T2* weighted GRE sequences) assessment of the brain. The screening MRI occurs as close to the beginning of the screening window as possible and at least 10 days prior to randomization on Day 1.

A lumbar puncture to obtain a CSF baseline sample is performed.

Amyloid-positron emission tomography (PET) imaging is performed in all participants. Participants may also participate in an optional exploratory assessment to evaluate changes in the brain as measured by translocator protein (TSPO)-PET.

Following the first IV infusion of study drug on Day 1 and discharge on Day 2, participants return to the study site as outpatients on Days 8, 15 and 22 (+2 days) for safety assessment follow-up, and on Day 29 (+1 day) to be administered the second dose of study drug.

Lumbar puncture to obtain CSF is performed on Day 50 and Day 64 (+2 days), or on a day determined by PK and PD data from previous single dose cohorts. Participants are followed for 16 weeks after the last infusion day.

Amyloid-PET imaging is performed in all participants on Day 106 (−2 days; +14 days). TSPO-PET imaging may be performed as an optional assessment on Day 36 (+10 days). A brain MRI is scheduled for all participants on Day 43 (+2 days).

### Eligibility Criteria

### Inclusion Criteria

Subjects that meet the following criteria are included in the SAD Phase of this study:
Participants in the SAD cohorts are healthy adults ages 18-65 years.

Subjects that meet the following criteria are included in the MD Phase of this study:
Adults ages 50-85 years.
Clinical diagnosis of probable Alzheimer's disease dementia based on National Institute on Aging Alzheimer's Association criteria.
Screening MMSE score of 16-28 points, inclusive.
Screening Clinical Dementia Rating-Global Score (CDR-GS) of 0.5, 1.0, or 2.0.
Positive amyloid-PET scan by qualitative read.
If already taking cholinesterase inhibitor and/or memantine therapy for Alzheimer's disease, on a stable dose for at least 4 weeks prior to screening, with no intent to initiate, discontinue, or alter the dose of any therapy for Alzheimer's disease for the duration of the study.

### Exclusion Criteria

Subjects are excluded from this study if they meet the following criteria:
Carriers of two copies of the minor allele rs12459419T.
History or presence of CNS or systemic autoimmune disorders including but not limited to rheumatoid arthritis, multiple sclerosis, lupus erythematosus, anti-phospholipid antibody syndrome, Behçet disease.
Dementia due to a condition other than Alzheimer's disease, including, but not limited to, Frontotemporal Dementia, Parkinson's disease, dementia with Lewy bodies, Huntington disease, or vascular dementia.
History or presence of clinically evident vascular disease potentially affecting the brain (e.g., clinically significant carotid, vertebral stenosis or plaque; aortic aneurysm;
intracranial aneurysm; cerebral hemorrhage; arteriovenous malformation) that has the potential to affect cognitive function.
History or presence of stroke within the past 2 years, or documented history of transient ischemic attack within the last 12 months.
History of severe, clinically significant (persistent neurologic deficit or structural brain damage) CNS trauma (e.g., cerebral contusion).
MRI evidence of
   More than two lacunar infarcts;
   Any territorial infarct >1 cm³; or
   Significant FLAIR hyperintense lesions in the cerebral white matter that may contribute to cognitive dysfunction.

### Study Duration

The duration of study participation for each participant in the SAD cohorts is about 16 weeks, including up to 4 weeks of screening, a single treatment on Day 1 and follow-up period, culminating in a final study completion visit on Day 85 (+5 days).

The duration of study participation for each participant in the MD cohort is about 26 weeks, including up to 6 weeks of screening, multiple treatments (two doses administered 4 weeks apart [q4w×2]) and a follow-up period, culminating in a final study completion visit on Day 141 (+5 days).

### Administration of Study Drug

Anti-CD33 antibody AB-64.1.2 is administered by intravenous (IV) infusion over about 60 minutes. The rate of infusion is adjusted in the event of an infusion related reaction. Placebo for IV infusion is commercially available normal saline.

### Study Outcome Assessments

### Pharmacokinetics Outcomes

Pharmacokinetic outcome measures for the study include:
Serum and CSF concentration, and PK parameters of anti-CD33 antibody AB-64.1.2 at specified time points.
Relationship between concentration or PK parameters for anti-CD33 antibody AB-64.1.2 and safety endpoints.
Relationship between concentration or PK parameters for anti-CD33 antibody AB-64.1.2 and activity or PD endpoints (relationship with activity is an endpoint only for the MD cohort i.e., patients with Alzheimer's disease).
Incidence of treatment-emergent antidrug antibodies (ADAs)

### Clinical Outcomes

Exploratory clinical outcome measures (for the MD cohort only, i.e., patients with Alzheimer's disease) include:
Clinical Dementia Rating (CDR) Sum of Boxes (CDR-SB) score (change after dosing relative to baseline).
Mini-Mental State Examination (MMSE) score (change after dosing relative to baseline).
Repeatable Battery for the Assessment of Neuropsychological Status (RBANS) score (change after dosing relative to baseline).

Washington University's CDR is a global assessment instrument that yields global scores (i.e., CDR-GS). The sum of boxes (i.e., CDR-SB) score is a detailed quantitative general index that provides more information than the CDR-GS in patients with mild dementia (O'Bryant et al., (2010) Arch Neurol, 67 (6): 746-49). The CDR characterizes 6 domains of cognitive and functional performance applicable to Alzheimer's disease and related dementias: memory, orientation, judgment and problem solving, community affairs, home and hobbies, and personal care. The necessary information to make each rating is obtained through a semi-structured interview of the patient and a reliable informant or collateral source (e.g., a caregiver).

The MMSE is a brief test used to screen for cognitive impairment. It is routinely used for estimating the severity of cognitive impairment and tracking cognitive changes in an individual over time. The MMSE assesses orientation (time and place), registration, attention and calculation, recent memory, language (naming, comprehension and repetition), and constructional praxis (copying a figure). The maximum total score is 30, with a higher score indicating better cognitive performance.

The RBANS is a collection of 12 subtests representing 5 neurocognitive domains: Immediate Memory, Visuospatial/Constructional, Language, Attention, and Delayed Memory. The raw scores from each subtest within a domain are converted to a summary score, or Index Score, for the domain by consulting normative data tables. The RBANS also provides an overall Index Score that summarizes the patient's overall level of performance on this measure.

### Pharmacodynamics Outcomes

Pharmacodynamics biomarkers are assessed, including the following:
Blood-based biomarkers:
   Cell surface expression of CD33.
   Soluble CD33 (sCD33) in plasma.
   Markers of neuroinflammation in blood.
   Cell surface expression of relevant biomarkers/antigens.
CSF-based biomarkers:
   sCD33.
   CSF biomarkers relevant to Alzheimer's disease, e.g., Abeta, Tau, p-Tau, neurofilament light chain [NF-L], neurogranin, and YKL-40.
   Other relevant markers of neuroinflammation.
Genetic markers relevant to the disease indication including the following:
   ApoE4.
   TREM2 variants, CD33 variants, TMEM106b variants, CLUSTERIN variants.
Imaging biomarkers (for MD cohort only, i.e., patients with Alzheimer's disease):
   Magnetic resonance imaging (MRI).
   Amyloid-positon emission tomography (PET) (in all Alzheimer's disease patients).
   Translocator protein (TSPO)-PET.
Analysis of exploratory biomarker endpoints for the study include:
   Changes in expression levels of cell surface CD33.
   Changes in levels of sCD33 in plasma and CSF after dosing relative to baseline concentration.
   Changes in expression of cell surface antigens.
   Relationship between biomarkers at baseline, including common and rare genetic variants, identified through whole genome sequencing (WGS) performed on deoxyribonucleic acid (DNA) extracted from blood, and safety, PK, activity, immunogenicity, or other biomarker endpoints (relationship with activity is an endpoint only for the MD patient cohort, i.e., patients with Alzheimer's disease).
   Changes in brain amyloid burden as assessed by Amyloid-PET in the MD patient cohort only, i.e., patients with Alzheimer's disease.
   Changes in brain inflammation as assessed by translocator protein (TSPO)-PET.
   Changes in markers of neuroinflammation and disease process in CSF and plasma.
Biomarkers analyzed by whole genome sequencing include the following:
   Apolipoprotein E4 (ApoE4).
   TREM2 variants, CD33 variants, TMEM106b variants, CLUSTERIN variants.

### Safety and Tolerability

The safety and tolerability endpoints of this study include:
Incidence, nature, severity and seriousness of adverse events (AEs) and treatment emergent adverse events (TEAEs).
Incidence of dose limiting adverse events (DLAEs), treatment discontinuations and dose reductions due to AEs.
Incidence of adverse events (AEs) of Special Interest (AESIs), including the following:
   New or worsening brain edema.
   New cerebral micro-hemorrhages.
   Grade 2 or higher AEs considered potentially CD33-mediated.
Mean changes in clinical laboratory tests from baseline over time; incidence of treatment emergent abnormal laboratory values and abnormal laboratory values reported as AEs.
Physical and neurologic examination abnormalities.
Mean change in vital signs from baseline over time and incidence of abnormal vital sign measurements.
Suicidal ideation, suicidal behavior, and self-injurious behavior without suicidal intent, as determined using the Sheehan-STS (for the MD patient cohort only).
Incidence of anti-drug antibodies (ADAs) during the study relative to the prevalence of ADAs at baseline (in SAD healthy adult participant cohorts and in the MD patient cohort).

### Statistical Methods

### Analysis Populations

The statistical analysis populations include the following:

Treatment received population: The treatment received population includes all randomized participants and is based on the treatment/dose level received.

Safety population: The safety population includes all randomized participants who receive any amount of study drug (anti-CD33 antibody AB-64.1.2 or placebo) and is based on the actual treatment/dose level received, if this differs to what the participant is randomized to.

PK population: The PK population includes all randomized participants who receive any amount of active study drug (anti-CD33 antibody AB-64.1.2) with sufficient data to determine at least one PK parameter. Participants who receive only placebo are excluded from the PK population.

PD population: The PD population includes all randomized participants who receive any amount of study drug (anti-CD33 antibody AB-64.1.2 or placebo) with results from baseline and from at least one post-baseline PD assessment, and is based on the actual treatment/dose level received, if this differs from what the participant was randomized to.

### Pharmacokinetics

Individual and mean anti-CD33 antibody AB-64.1.2 concentration-time data in blood (e.g., serum or plasma) or CSF is tabulated and plotted by cohort/dose level. PK parameters are computed from the individual anti-CD33 antibody AB-64.1.2 concentrations using a non-compartmental approach. The PK parameters estimated include:
Maximum drug concentration (Cₘₐₓ).
Time to reach Cₘₐₓ (Tₘₐₓ).
Area under the drug concentration-time curve from time zero to the last quantifiable concentration (AUC₍₀₋ₗₐₛₜ₎).
Area under the drug concentration-time curve from time zero to infinity (AUC_{(0-inf)}) calculated as the sum of AUC₍₀₋ₗₐₛₜ₎ plus the last measurable concentration divided by elimination rate constant (kₑₗ).
Areas under the drug concentration-time curve over the inter-dosing interval (AUCₜₐᵤ) where tau is the time over the inter-dosing interval. Calculated for the MD cohort only.
Apparent terminal elimination rate constant (kₑₗ) calculated by linear regression of the terminal linear portion of the log concentration vs. time curve.
Apparent terminal half-life (t_{1/2}).
Apparent total body clearance after extravascular administration (SAD cohorts: CL; MD cohort CL_{SS}), calculated as Dose/AUC_{0-inf} for single/first dose and Dose/AUCₜₐᵤ after multiple dose administrations.
Apparent total volume of distribution at the terminal phase after extravascular administration (SAD cohorts: V_{z}; MD cohort: V_{zss}), calculated as Dose/(kₑₗ×AUC_{0-inf}) after single/first dose and Dose/(kₑₗ×AUCₜₐᵤ) after multiple dose administrations.

Values for kₑₗ, t_{1/2}, AUC_{0-inf}, CL or V_{z} are not reported for cases that fail to exhibit a terminal log-linear phase in the concentration versus time profile.

Estimates for PK parameters are tabulated and summarized by descriptive statistics (mean, standard deviation [SD], median, minimum, and maximum, coefficient of variation [CV %], geometric mean and 90% confidence interval [CI], and geometric CV %).

Potential correlations of relevant PK parameters with dose, demographics, safety (including QT changes), and PD measures are explored. Additional modelling, including population PK analysis, to characterize these correlations is performed.

### Exploratory Clinical Outcomes

Individual exploratory clinical outcome measures for CDR-SB, MMSE and RBANS are presented in a data listing for all participants (for the MD cohort only, i.e. patients with Alzheimer's disease). CDR-SB, MMSE and RBANS are summarized by time point and treatment group (active or placebo) and a summary of change from baseline by treatment group is presented.

### Pharmacodynamics and Exploratory Biomarkers

All individual PD biomarker data are presented in data listings and summarized by nominal sampling time point, treatment group and cohort with descriptive statistics (e.g., number of non-missing observations, arithmetic mean, SD, median, minimum, maximum and % CV). The number of values below the limit of quantitation (BLQ) are presented. Observed change from baseline and percent changes from baseline for PD biomarker parameters are summarized separately for the single dosing cohorts and the multiple dosing cohort.

Exploratory analyses of biomarkers are conducted to evaluate the effect of anti-CD33 antibody AB-64.1.2 on exploratory biomarkers. In addition, exploratory biomarkers are analyzed before and after dosing with anti-CD33 antibody AB-64.1.2 to determine the relationship between PK exposure and biomarker levels.

### Example 2: Results from a Phase 1 Study of Single and Multiple Doses of Anti-CD33 Antibody AB-64.1.2 in Healthy Participants and in Participants with Mild to Moderate Alzheimer's Disease

This Example provides results of the Phase 1 study described in Example 1, which assessed the safety (including immunogenicity) and tolerability, pharmacokinetics (PK), and pharmacodynamics (PD) of anti-CD33 antibody AB-64.1.2 when administered as single ascending doses in healthy participants, and as multiple doses in patients with mild to moderate Alzheimer's disease.

As described in detail in Example 1, this study included two parts, a single ascending dose (SAD) phase in healthy volunteers (Part 1), and a multiple dose (MD) phase in participants with mild-to-moderate Alzheimer's disease (Part 2).

During Part 1, healthy human volunteers (HVs) were administered single doses of anti-CD33 antibody AB-64.1.2, ranging from 0.05 mg/kg to 60.0 mg/kg (Cohorts A-H). In SAD Cohorts A, B and C, three HVs were sequentially enrolled into 3 single-participant cohorts and treated with anti-CD33 antibody AB-64.1.2 at doses of 0.05 mg/kg, 0.2 mg/kg, or 0.6 mg/kg, respectively. In SAD cohorts D through H, cohorts of eight HVs (6 active [anti-CD33 antibody AB-64.1.2]; 2 placebo) were sequentially enrolled and treated with anti-CD33 antibody AB-64.1.2 or placebo at doses ranging from 1.6 to 60 mg/kg. For cohorts E through H, lumbar punctures (LPs) were performed at screening and on days 8 and 18 to obtain cerebrospinal fluid (CSF) samples to measure PK and exploratory biomarker levels. SAD cohorts were followed for safety for 12 weeks after study drug administration.

During Part 2, participants with mild-to-moderate Alzheimer's disease were enrolled into Cohort I and received 2 doses of anti-CD33 antibody AB-64.1.2 at a dose of 15 mg/kg or placebo (10 active; 2 placebo), administered 4 weeks apart (i.e., q4w×2). LPs were performed at screening and at 2 post-dose time points for each participant (days 36, 43, 50, or 64). The MD cohort was followed for 16 weeks after the last dose of study drug.

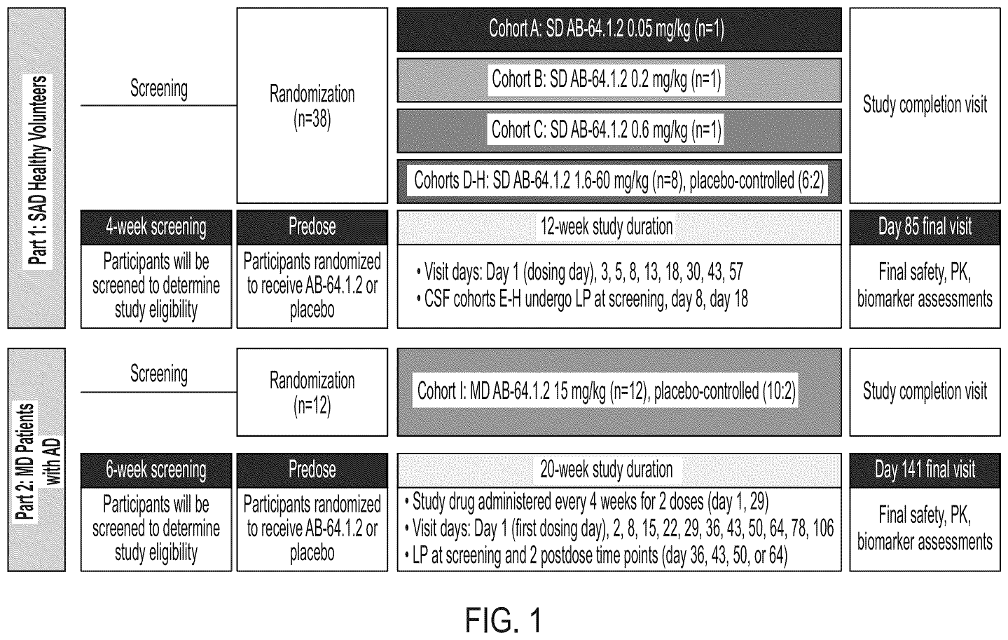

An overview of the design of the study is provided in FIG. 1.

### Results

### Study Participants

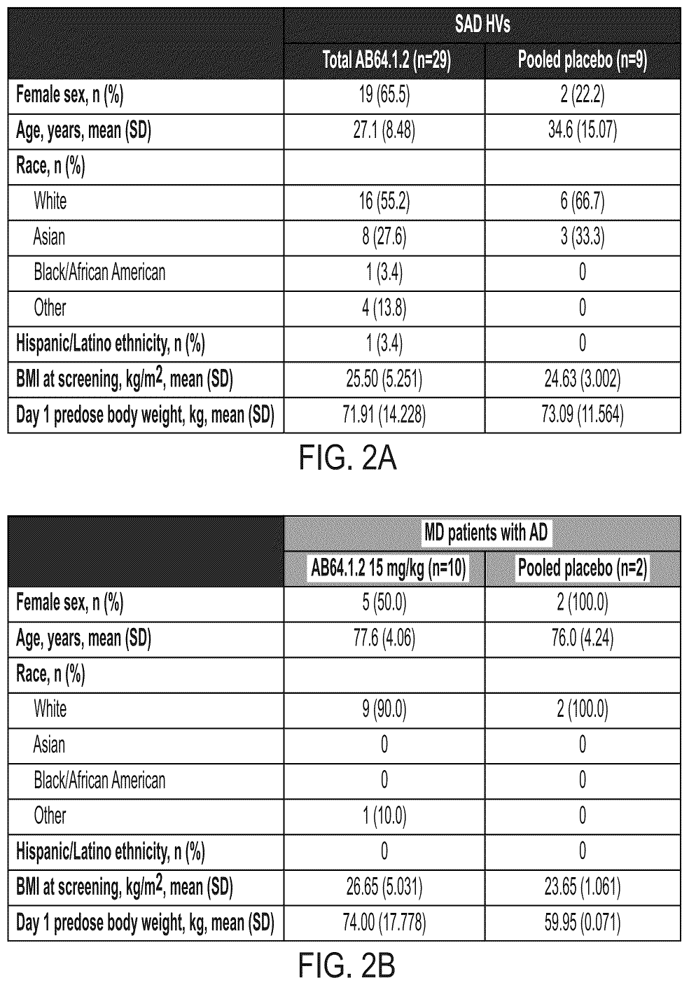

A total of 38 HVs were enrolled in Part 1 of this study. Twenty-nine participants received anti-CD33 antibody AB-64.1.2, and nine participants received placebo. See, FIG. 2A. The actual enrollment for Cohort H (the 60 mg/kg dose cohort) included two participants who received anti-CD33 antibody AB-64.1.2, and one participant who received placebo. Among the HVs, 3 participants treated with anti-CD33 antibody AB-64.1.2 withdrew from the study (one participant in the 30 mg/kg group and one participant in the 60 mg/kg group withdrew consent; and one participant in the 0.05 mg/kg group discontinued the study due to relocation).

A total of 12 participants with Alzheimer's disease were enrolled in Part 2 of this study. Ten participants received anti-CD33 antibody AB-64.1.2 at a dose of 15 mg/kg, and two participants received placebo. See, FIG. 2B. All participants with Alzheimer's disease completed the study.

An overview of baseline demographics for participants in Parts 1 and 2 of the study is provided in FIGS. 2A-2B.

### Safety

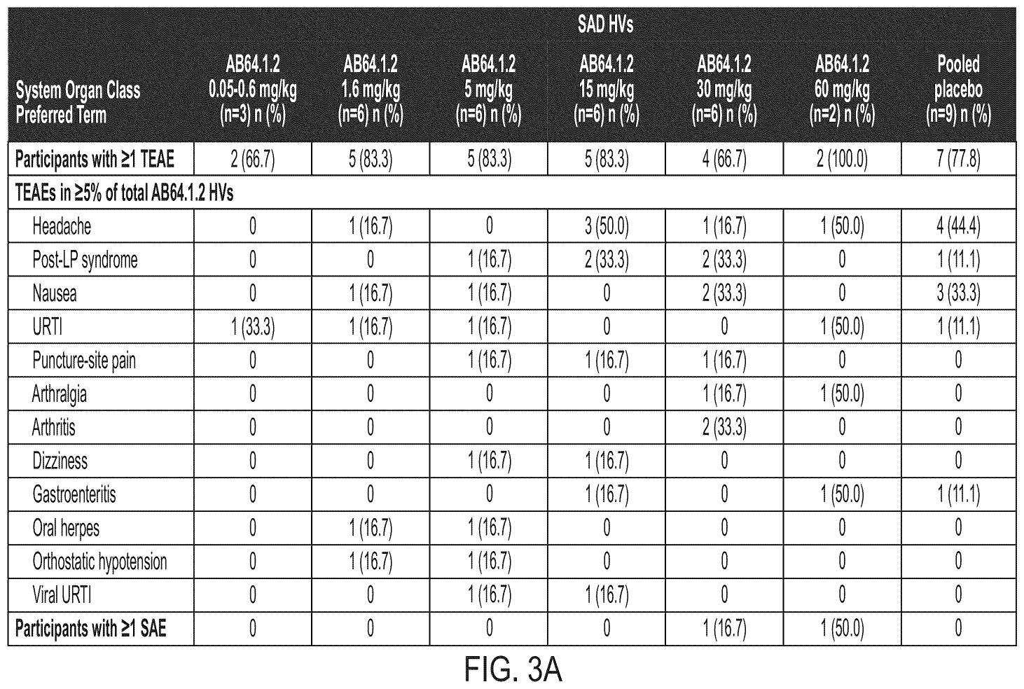

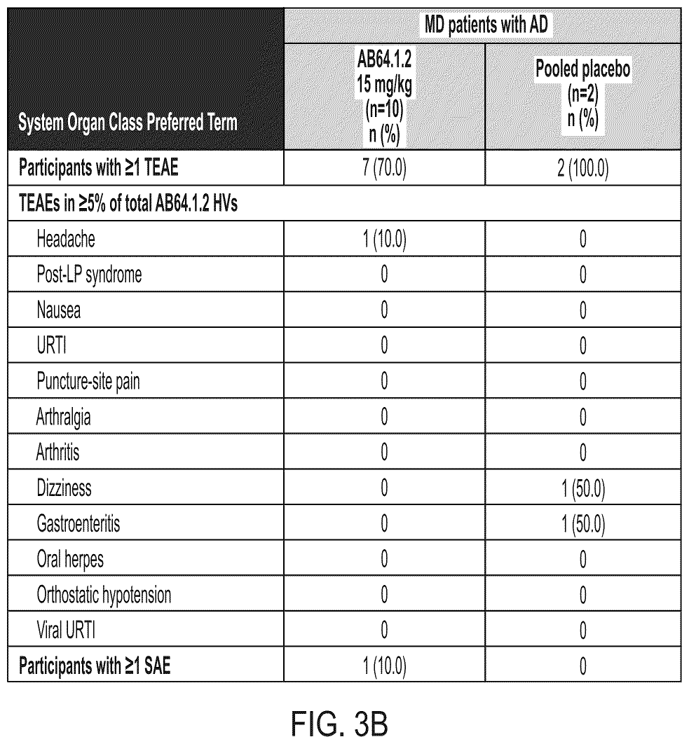

In Part 1 of the study, 79.3% of HVs treated with anti-CD33 antibody AB-64.1.2 experienced treatment-emergent adverse events (TEAEs), compared with 77.8% of HVs in the pooled placebo group. Dose levels up to 15 mg/kg were well tolerated in HVs, with immune-related AEs seen at higher dose levels (30 and 60 mg/kg). The most frequently reported TEAEs in HVs receiving anti-CD33 antibody AB-64.1.2 were headache (20.7%), post-LP syndrome (i.e., headache after LP; 17.2%), nausea (13.8%), upper respiratory tract infection (13.8%), and puncture-site pain (10.3%). 2 HVs experienced SAEs considered related to the study drug: one participant (administered anti-CD33 antibody AB-64.1.2 at a dose of 30 mg/kg) reported aseptic arthritis of the hip 15 days after receiving study drug, and one participant (administered anti-CD33 antibody AB-64.1.2 at a dose of 60 mg/kg) reported severe hypersensitivity characterized by rash, fever, thrombocytopenia, and elevated C-reactive protein 9 days after receiving study drug. The adverse event of hypersensitivity in the 60 mg/kg cohort (Cohort H) met the protocol-defined criteria for a dose-limiting adverse event (DLAE) and led to discontinuation of further enrollment in this cohort. See, FIG. 3A.

Among the MD cohort of patients with Alzheimer's disease in Part 2 of the study, 70.0% of participants treated with anti-CD33 antibody AB-64.1.2 (15 mg/kg) experienced TEAEs, compared with 100.0% of patients in the placebo group. One participant with Alzheimer's disease in the anti-CD33 antibody AB-64.1.2 group experienced an SAE considered unrelated to study treatment (cerebrovascular accident). See, FIG. 3B.

No TEAEs leading to study drug withdrawal or early discontinuation occurred during the study among the SAD HVs or MD patients with AD

### Pharmacokinetics

### Serum

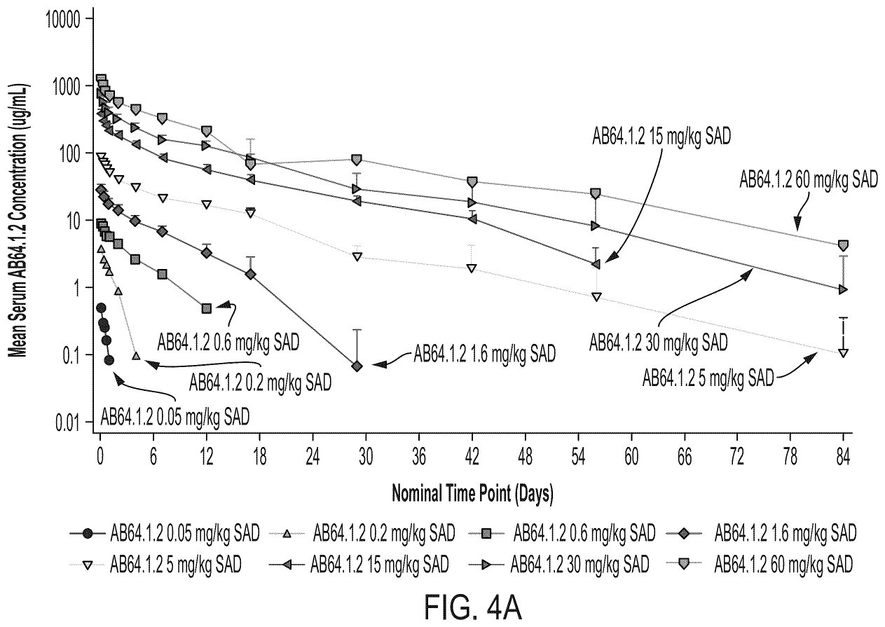

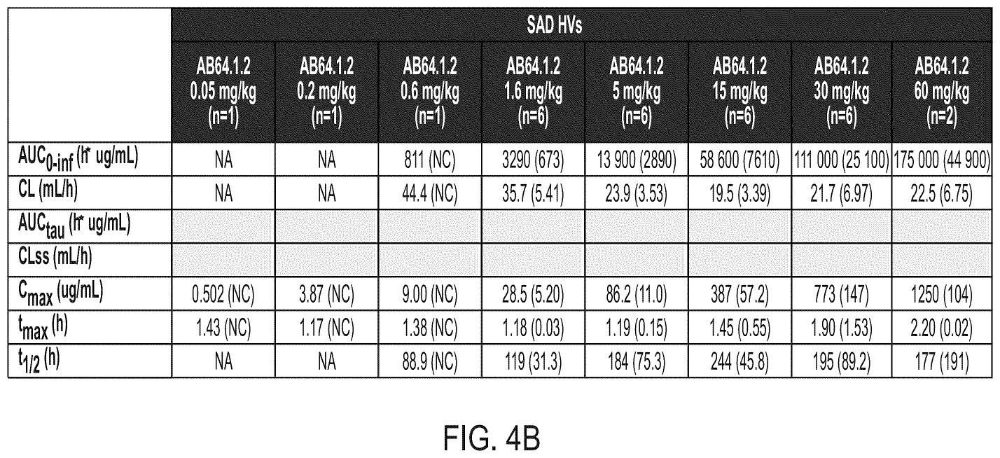

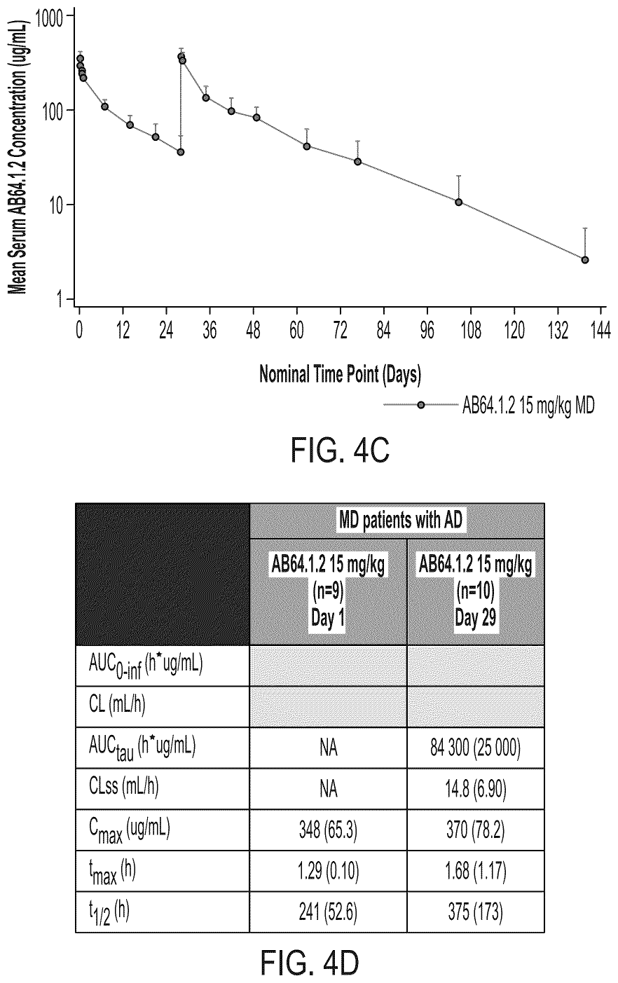

After a single IV infusion of anti-CD33 antibody AB-64.1.2 at doses ranging from 0.05 to 60 mg/kg in HVs in Part 1 of the study, mean serum concentrations of anti-CD33 antibody AB-64.1.2 were consistently elevated in a dose-dependent manner (FIG. 4A). Anti-CD33 antibody AB-64.1.2 had an approximately dose-proportional Cₘₐₓ and AUC_{0-inf} from 15 mg/kg to 30 mg/kg, with nonlinear exposures between dose levels of 1.6 mg/kg to 15 mg/kg (FIG. 4B).

In Part 2 of the study (15 mg/kg, 2 doses, q4w), Cₘₐₓ was similar to that of HVs in the anti-CD33 antibody AB-64.1.2 15 mg/kg group from Part 1 (FIGS. 4C-4D). However, participants with Alzheimer's disease showed slightly slower clearance compared to HVs, which was reflected by a longer half-life at day 29 in patients with Alzheimer's disease when compared with day 1 in HVs.

### Cerebrospinal Fluid

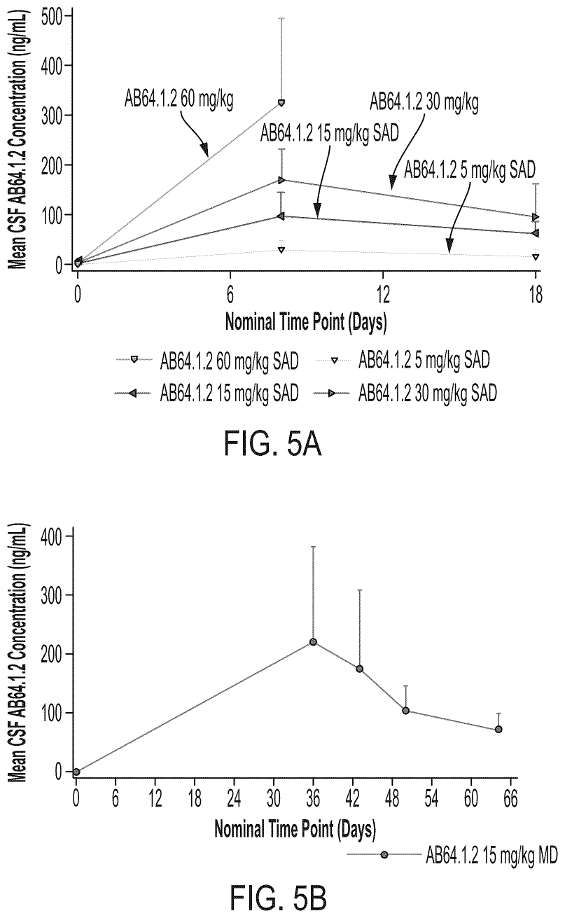

For both SAD HVs and MD participants in Parts 1 and 2 of the study, mean cerebrospinal fluid (CSF) concentration-time profiles for anti-CD33 antibody AB-64.1.2 demonstrated central nervous system (CNS) penetrance of anti-CD33 antibody AB-64.1.2 (FIGS. 5A-5B), with geometric mean partition coefficients (CSF over serum) of 0.1% to 0.2%.

### Biomarkers

### CD33 Expression on Monocytes

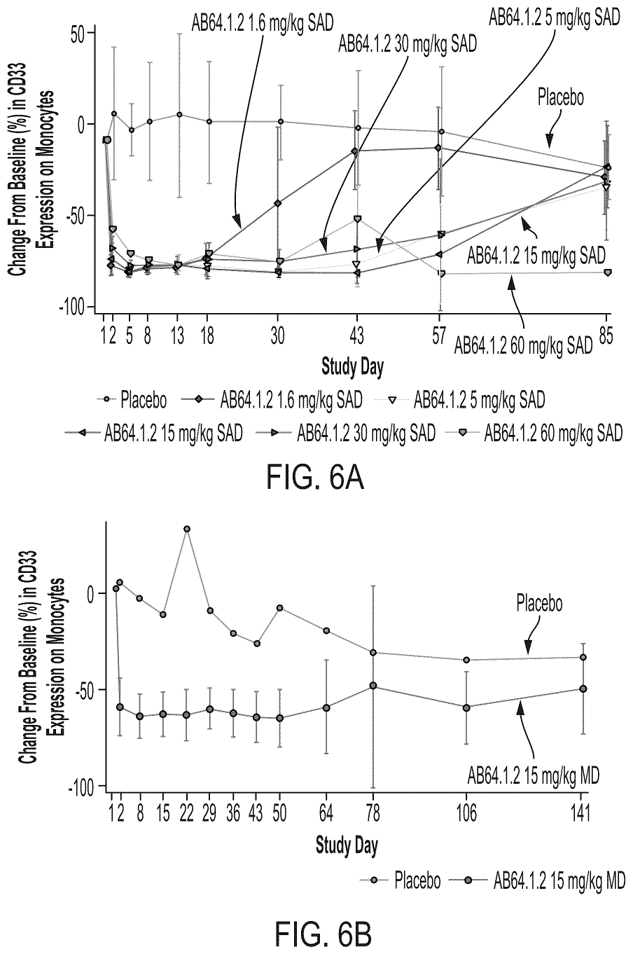

Administration of single doses of anti-CD33 antibody AB-64.1.2 in Part 1 of the study reduced CD33 expression on peripheral monocytes in HVs at dose levels of 1.6 mg/kg and greater (FIG. 6A). Similarly, administration of multiple doses of anti-CD33 antibody AB-64.1.2 in Part 2 of the study reduced CD33 expression on peripheral monocytes in patients with Alzheimer's disease, and the reduction was sustained for nearly the entire study duration (FIG. 6B)

### Soluble CD33 Levels in CSF

The levels of soluble CD33 (sCD33) in CSF were assessed using a Quantitative Liquid Chromatography Multiple-Reaction Monitoring Mass Spectrometry (LCMRM/MS) assay for CD33 protein. Each CSF sample analysis batch included a calibration curve and a set of Quality Control (QC) samples. Calibration standards were prepared at eight levels covering the assay range by spiking recombinant CD33 protein in surrogate matrix. QC samples were prepared at three levels covering the assay range by spiking recombinant CD33 protein in pooled human CSF from healthy donors. Each sample analysis batch also included QC samples at endogenous level (non-spiked) and blank samples. Standards, blanks, QC samples and study samples were processed as follows: digestion with trypsin, spiking with a Stable Isotope Labeled (SIL) internal standard peptide, and desalting using Solid-Phase Extraction (SPE) sorbent matrix. Following processing, all standards, QC samples and study samples were analyzed using an LC-MRM/MS assay which monitored transitions from endogenous target peptide (FAGAGVTTER [SEQ ID NO: 27]) and its SIL counterpart. The concentration of sCD33 in study samples and QC samples was determined by calculating the ratio of endogenous target peptide and SIL peptide and back-calculating the value onto the calibration curve.

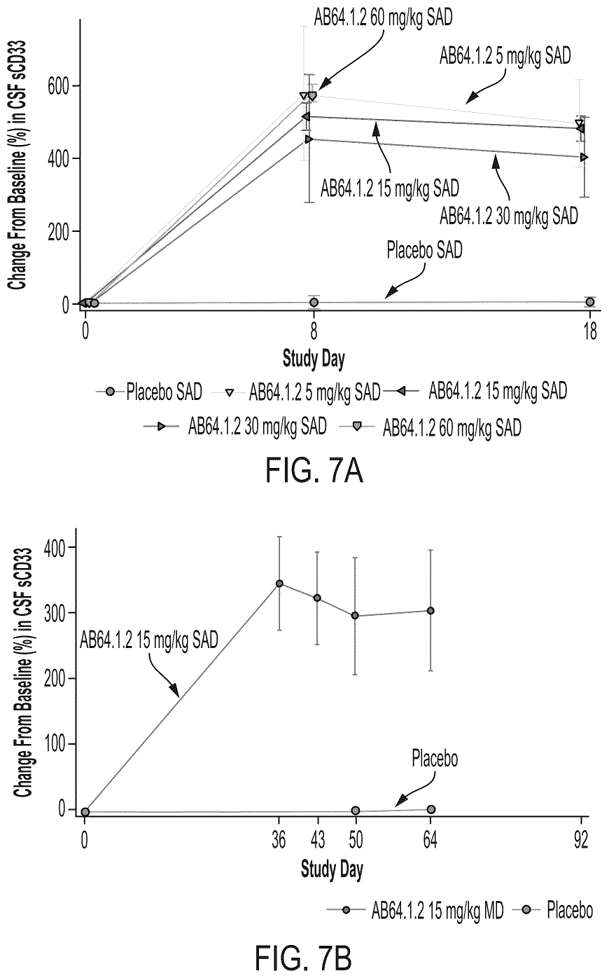

As shown in FIG. 7A, administration of single doses of anti-CD33 antibody AB-64.1.2 in Part 1 of the study increased CSF levels of sCD33 in HVs, providing evidence of target engagement of the antibody in the CNS (FIG. 7A). Similarly, administration of multiple doses of anti-CD33 antibody AB-64.1.2 in Part 2 of the study increased CSF levels of SCD33 in patients with Alzheimer's disease, and sCD33 levels remained elevated from baseline levels at study day 64, also providing evidence of target engagement of the antibody in the CNS of patients with Alzheimer's disease (FIG. 7B)

### CONCLUSIONS

The safety results presented in this Example show that anti-CD33 antibody AB-64.1.2 was generally safe and well tolerated in healthy volunteers and patients with Alzheimer's disease up to and including doses of 15 mg/kg. In addition, the biomarker results described in this Example demonstrated target engagement of anti-CD33 antibody AB-64.1.2 in both the blood and CNS compartments across the tolerated dose range.

1.-2. (canceled)

3. A method of assessing activity of an anti-CD33 antibody in an individual having a disease or injury and being treated with an anti-CD33 antibody, the method comprising:
(a) determining a level of soluble CD33 protein (sCD33) in a sample obtained from an individual having a disease or injury and being treated with an anti-CD33 antibody, wherein the sample is obtained after the individual has received a dose of the anti-CD33 antibody;
(b) assessing activity of the anti-CD33 antibody in the individual based on the level of sCD33 in the sample, wherein the anti-CD33 antibody is determined to be active in the individual if the level of sCD33 in the sample is increased as compared to a level of sCD33 in a sample obtained from the individual prior to the start of treatment with the anti-CD33 antibody.

4. A method of monitoring treatment of an individual having a disease or injury and being treated with an anti-CD33 antibody, the method comprising:
(a) determining a level of soluble CD33 protein (sCD33) in a sample obtained from an individual having a disease or injury and being treated with an anti-CD33 antibody, wherein the sample is obtained at a first time point after the individual has received a dose of the anti-CD33 antibody;
(b) determining a level of sCD33 in one or more additional samples obtained from the individual at a one or more time points after the first time point;
(c) assessing activity of the anti-CD33 antibody in the individual based on the level of sCD33 in the sample and/or in the one or more additional samples, wherein the anti-CD33 antibody is determined to be active in the individual if the level of sCD33 in the sample and/or in the one or more additional samples is increased as compared to a level of sCD33 in a sample obtained from the individual prior to the start of treatment with the anti-CD33 antibody.

5.-17. (canceled)

18. A method for adjusting treatment of an individual having a disease or injury and being treated with an anti-CD33 antibody, the method comprising:
(a) determining a level of soluble CD33 protein (sCD33) in a sample obtained from an individual having a disease or injury and being treated with one or more doses of an anti-CD33 antibody, wherein the sample is obtained after the individual has received a dose of the anti-CD33 antibody, wherein:
(i) presence of an increase in the level of sCD33 in the sample indicates that treatment with the anti-CD33 antibody should continue, or
(ii) absence of an increase in the level of sCD33 in the sample indicates that treatment with the anti-CD33 antibody should continue at a higher dose of the anti-CD33 antibody as compared to the one or more doses of anti-CD33 antibody being administered to the individual,
wherein the increase in the level of sCD33 in the sample is determined as compared to a level of sCD33 in a sample obtained from the individual prior to administration of the anti-CD33 antibody; and
(b) adjusting the treatment of the individual based, at least in part, on determining presence or absence of the increase in the level of sCD33 in the sample.

19.-23. (canceled)

24. The method of claim 3, wherein the sample or the one or more additional samples obtained from the individual are a sample of blood, plasma, serum, or cerebrospinal fluid.

25.-28. (canceled)

29. The method of claim 3, further comprising obtaining the sample, or the one or more samples, from the individual.

30. (canceled)

31. The method of claim 3, wherein the disease or injury is selected from the group consisting of dementia, frontotemporal dementia, Alzheimer's disease, vascular dementia, mixed dementia, and taupathy disease.

32. The method of claim 31, wherein the disease or injury is Alzheimer's disease.

33.-38. (canceled)

39. The method of claim 3, wherein the individual is a human.

40.-43. (canceled)

44. The method of claim 3, wherein the anti-CD33 antibody binds specifically to a human CD33 protein.

45. (canceled)

46. The method of claim 3, wherein the anti-CD33 antibody is a monoclonal antibody.

47.-48. (canceled)

49. The method of claim 3, wherein the anti-CD33 antibody is of the IgG class, the IgM class, or the IgA class.

50. The method of claim 49, wherein the anti-CD33 antibody is of the IgG class and has an IgG1, IgG2, IgG3, or IgG4 isotype.

51. The method of claim 50, wherein the anti-CD33 antibody has an IgG4 isotype, and wherein the antibody comprises an S228P amino acid substitution at residue position 228, an F234A amino acid substitution at residue position 234, and an L235A amino acid substitution at residue position 235, wherein the numbering of the residue position is according to EU numbering.

52. The method of claim 50, wherein the anti-CD33 antibody has an IgG2 isotype.

53. The method of claim 50, wherein the anti-CD33 antibody comprises one or more amino acid substitutions in the Fc region at a residue position selected from the group consisting of: C127S, L234A, L234F, L235A, L235E, S267E, K322A, L328F, A330S, P331S, E345R, E430G, S440Y, and any combination thereof, wherein the numbering of the residues is according to EU or Kabat numbering.

54. The method of claim 50, wherein:
the Fc region comprises an amino acid substitution at position E430G, wherein the numbering of the residue position is according to EU numbering;
the Fc region comprises an amino acid substitution at positions L243A, L235A, and P331A, wherein the numbering of the residue position is according to EU numbering;
the Fc region comprises an amino acid substitution at positions L243A, L235A, P331A, and E430G, wherein the numbering of the residue position is according to EU numbering;
the Fc region comprises an amino acid substitution at positions K322A and E430G, wherein the numbering of the residue position is according to EU numbering;
the Fc region comprises an amino acid substitution at positions P331S and E430G, wherein the numbering of the residue position is according to EU numbering;
the Fc region comprises an amino acid substitution at positions A330S, P331S, and E430G, wherein the numbering of the residue position is according to EU numbering;
the Fc region comprises an amino acid substitution at positions K322A, A330S, and P331S, wherein the numbering of the residue position is according to EU numbering;
the Fc region comprises an amino acid substitution at positions K322A, P331S, and E430G, wherein the numbering of the residue position is according to EU numbering;
the Fc region comprises an amino acid substitution at position E430G, wherein the numbering of the residue position is according to EU numbering;
the Fc region comprises an amino acid substitution at positions A330S, P331S, and E430G, wherein the numbering of the residue position is according to EU numbering;
the Fc region comprises an amino acid substitution at positions S267E and L328F, wherein the numbering of the residue position is according to EU numbering;
the Fc region comprises an amino acid substitution at position C127S, wherein the numbering of the residue position is according to EU numbering;
the Fc region comprises an amino acid substitution at positions E345R, E430G and S440Y, wherein the numbering of the residue position is according to EU numbering;
the Fc region comprises an amino acid substitution at position P331S, wherein the numbering of the residue position is according to EU numbering; or
the Fc region comprises an amino acid substitution at positions L234A, L235A, P331S, wherein the numbering of the residue positions is according to EU numbering.

55. The method of claim 50, wherein:
(a) the anti-CD33 antibody has a human IgG1 isotype and comprises one or more amino acid substitutions in the Fc region at a residue position selected from the group consisting of: N297A, D265A, D270A, L234A, L235A, G237A, P238D, L328E, E233D, G237D, H268D, P271G, A330R, C226S, C229S, E233P, L234V, L234F, L235E, P331S, S267E, L328F, A330L, M252Y, S254T, T256E, N297Q, P238S, P238A, A327Q, A327G, P329A, K322A, T394D, and any combination thereof, wherein the numbering of the residues is according to EU numbering, or comprises an amino acid deletion in the Fc region at a position corresponding to glycine 236;
(b) the anti-CD33 antibody has a human IgG1 isotype and comprises an IgG2 isotype heavy chain constant domain 1 (CH1) and hinge region, wherein the IgG2 isotype CH1 and hinge region comprises the amino acid sequence of ASTKGPSVFP LAPCSRSTSE STAALGCLVK DYFPEPVTVS WNSGALTSGVHTFPAVLQSS GLYSLSSVVT VPSSNFGTQT YTCNVDHKPS NTKVDKTVERKCCVECPPCP (SEQ ID NO: 26), and wherein the antibody Fc region comprises a S267E amino acid substitution, or a L328F amino acid substitution, or both, and/or a N297A or N297Q amino acid substitution, wherein the numbering of the residues is according to EU numbering;
(c) the anti-CD33 antibody has a human IgG2 isotype and comprises one or more amino acid substitutions in the Fc region at a residue position selected from the group consisting of: P238S, V234A, G237A, H268A, H268Q, V309L, A330S, P331S, C214S, C232S, C233S, S267E, L328F, M252Y, S254T, T256E, H268E, N297A, N297Q, A330L, and any combination thereof, wherein the numbering of the residues is according to EU numbering;
(d) the anti-CD33 antibody has a human IgG4 isotype and comprises one or more amino acid substitutions in the Fc region at a residue position selected from the group consisting of: L235A, G237A, S228P, L236E, S267E, E318A, L328F, M252Y, S254T, T256E, E233P, F234V, L234A/F234A, S228P, S241P, L248E, T394D, N297A, N297Q, L235E, and any combination thereof, wherein the numbering of the residues is according to EU numbering; or
(e) the anti-CD33 antibody has a hybrid IgG2/4 isotype, and wherein the antibody comprises an amino acid sequence comprising amino acids 118 to 260 of human IgG2 and amino acids 261 to 447 of human IgG4, wherein the numbering of the residues is according to EU or Kabat numbering.

56. The method of claim 3, wherein the anti-CD33 antibody is an antibody fragment.

57.-62. (canceled)

63. The method of claim 3, wherein the anti-CD33 antibody has a dissociation constant (K_{D}) for human CD33 that ranges from about 2 nM to about 200 pM and wherein the Kp is determined by BioLayer Interferometry.

64. The method of claim 3, wherein the anti-CD33 antibody reduces cell surface levels of a CD33 protein.

65. The method of claim 64, wherein the CD33 protein is expressed on the surface of human dendritic cells or monocytes.

66. The method of claim 3, wherein the anti-CD33 antibody reduces cell surface levels of CD33 in vitro.

67. The method of claim 66, wherein the anti-CD33 antibody reduces cell surface levels of CD33 in vitro with a half maximal effective concentration (EC50) that is less than 150 pM, or less than 40 pM, as measured by flow cytometry.

68. (canceled)

69. The method of claim 3, wherein the anti-CD33 antibody increases expression of one or more disease-associated microglia (DAM) markers, inhibits cell surface clustering of CD33, and/or increases phagocytosis by microglia.

70.-75. (canceled)

76. The method of claim 3, wherein the anti-CD33 antibody inhibits interaction between a human or mammalian CD33 protein and one or more CD33 ligands.

77. The method of claim 3, wherein the anti-CD33 antibody comprises:
(a) a heavy chain variable region comprising: an HVR-H1 comprising the amino acid sequence GYTFTDYNLH (SEQ ID NO: 1), or an amino acid sequence with at least about 90% homology to the amino acid sequence of SEQ ID NO: 1; an HVR-H2 comprising the amino acid sequence FIYPSNRITG (SEQ ID NO: 2), or an amino acid sequence with at least about 90% homology to the amino acid sequence of SEQ ID NO: 2; and an HVR-H3 comprising the amino acid sequence SDVDYFDY (SEQ ID NO: 3), or an amino acid sequence with at least about 90% homology to the amino acid sequence of SEQ ID NO: 3; and
(b) a light chain variable region comprising: an HVR-L1 comprising the amino acid sequence RASQSVSTSTYSYMH (SEQ ID NO: 4), or an amino acid sequence with at least about 90% homology to the amino acid sequence of SEQ ID NO: 4; an HVR-L2 comprising the amino acid sequence YASNLES (SEQ ID NO: 5), or an amino acid sequence with at least about 90% homology to the amino acid sequence of SEQ ID NO: 5, and an HVR-L3 comprising the amino acid sequence QHSWEIPLT (SEQ ID NO: 6), or an amino acid sequence with at least about 90% homology to the amino acid sequence of SEQ ID NO: 6.

78. The method of claim 3, wherein the anti-CD33 antibody comprises a heavy chain variable region comprising the amino acid sequence of SEQ ID NO: 7, and a light chain variable region comprising the amino acid sequence of SEQ ID NO: 8.

79.-80. (canceled)

81. The method of claim 3, wherein the anti-CD33 antibody comprises a heavy chain comprising the amino acid sequence of SEQ ID NO: 9 or SEQ ID NO: 10, and a light chain comprising the amino acid sequence of SEQ ID NO: 11.

82.-86. (canceled)

87. The method of claim 3, wherein the anti-CD33 antibody comprises:
(a) a heavy chain variable region comprising: an HVR-H1 comprising the amino acid sequence NYEMN (SEQ ID NO: 12), or an amino acid sequence with at least about 90% homology to the amino acid sequence of SEQ ID NO: 12; an HVR-H2 comprising the amino acid sequence EIRLKSNNYVTNYAASVKG (SEQ ID NO: 13), or an amino acid sequence with at least about 90% homology to the amino acid sequence of SEQ ID NO: 13; and an HVR-H3 comprising the amino acid sequence AGYYVPFAY (SEQ ID NO: 14), or an amino acid sequence with at least about 90% homology to the amino acid sequence of SEQ ID NO: 14; and
(b) a light chain variable region comprising: an HVR-L1 comprising the amino acid sequence TLSSQHSTYTIE (SEQ ID NO: 15), or an amino acid sequence with at least about 90% homology to the amino acid sequence of SEQ ID NO: 15; an HVR-L2 comprising the amino acid sequence LKKEGSHSTGD (SEQ ID NO: 16), or an amino acid sequence with at least about 90% homology to the amino acid sequence of SEQ ID NO: 16, and an HVR-L3 comprising the amino acid sequence GVGHTIKEQFVYV (SEQ ID NO: 17), or an amino acid sequence with at least about 90% homology to the amino acid sequence of SEQ ID NO: 17.

88. The method of claim 3, wherein the anti-CD33 antibody comprises a heavy chain variable region comprising the amino acid sequence of SEQ ID NO: 18, and a light chain variable region comprising the amino acid sequence of SEQ ID NO: 19.

89.-90. (canceled)

91. The method of claim 3, wherein the anti-CD33 antibody comprises a heavy chain comprising the amino acid sequence of SEQ ID NO: 20 or 21, and a light chain comprising the amino acid sequence of SEQ ID NO: 22.

92. The method of claim 3, wherein the sample or the one or more additional samples obtained from the individual are a cerebrospinal fluid sample, and wherein sCD33 levels in the sample or in the one or more additional samples obtained from the individual are increased by at least about 100%, at least about 125%, at least about 150%, at least about 175%, at least about 200%, at least about 225%, at least about 250%, at least about 275%, at least about 300%, at least about 325%, at least about 350%, at least about 375%, at least about 400%, at least about 425%, at least about 450%, at least about 475%, at least about 500%, at least about 525%, at least about 550%, at least about 575%, at least about 600%, or more, as compared to a level of sCD33 in a sample obtained from the individual prior to administration of the anti-CD33 antibody.

93. The method of claim 92, wherein the increase in the level of sCD33 is present for at least about 8 days, at least about 18 days, at least about 36 days, at least about 43 days, at least about 50 days, or at least about 64 days after administration of a dose of the anti-CD33 antibody.

94. The method of claim 3, further comprising measuring a concentration of the anti-CD33 antibody in a sample obtained from the individual after the individual has received one or more doses of the anti-CD33 antibody.

95.-96. (canceled)

97. The method of claim 3, further comprising measuring expression of a CD33 protein on the surface of one or more cells in a sample obtained from the individual after the individual has received one or more doses of the anti-CD33 antibody.

98.-100. (canceled)

101. The method of claim 3, wherein the anti-CD33 antibody reduces cell surface levels of a CD33 protein on one or more monocytes in the individual by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, as compared to cell surface levels of the CD33 protein on one or more monocytes prior to administration of the anti-CD33 antibody.

102. The method of claim 101, wherein the reduction of cell surface levels of the CD33 protein is present for at least about 1 day, at least about 2 days, at least about 5 days, at least about 8 days, at least about 13 days, at least about 15 days, at least about 22 days, at least about 29 days, at least about 30 days, at least about 36 days, at least about 43 days, at least about 50 days, at least about 57 days, at least about 64 days, at least about 78 days, at least about 106 days, or at least about 141 days after administration of a dose of the anti-CD33 antibody.

103.-110. (canceled)

111. The method of claim 3, wherein the anti-CD33 antibody increases levels of sCD33.

112. The method of claim 3, further comprising determining the level of sCD33 in the sample obtained from the individual prior to administration of the anti-CD33 antibody or prior to the start of treatment with the anti-CD33 antibody.

113. The method of claim 3, further comprising administering the dose of the anti-CD33 antibody to the individual.
